(19) United States
(12) Patent Application Publication
(10) Pub. No.: US 2023/0085549 A1
(43) Pub. Date: Mar. 16, 2023

(54) Dosing Regimens Associated With Extended Release Paliperidone Injectable Formulations

(71) Applicant: Janssen Pharmaceutica NV, Beerse (BE)

(72) Inventor: Srihari Gopal, Belle Mead, NJ (US)

(21) Appl. No.: 17/887,821

(22) Filed: Aug. 15, 2022

Related U.S. Application Data
(60) Provisional application No. 63/238,772, filed on Aug. 30, 2021.

(51) Int. Cl. A61K 31/519; A61P 25/18; A61K 9/00

(52) U.S. Cl. CPC A61K 31/519; A61P 25/18; A61K 9/0019

(57) ABSTRACT

The present invention provides a method of treating patients with long acting injectable paliperidone palmitate formulations. Also provided are pharmaceutical products containing long acting injectable paliperidone palmitate formulations, instructions for use of the long acting injectable paliperidone palmitate formulations, and methods for selling a drug product containing long acting injectable paliperidone palmitate formulations.

### CROSS-REFERENCE TO RELATED APPLICATIONS

This application claims the benefit of U.S. Provisional Patent Application No. 63/238,772, filed Aug. 30, 2021, the disclosure of which is incorporated by reference herein.

### FIELD OF THE INVENTION

This invention relates to pharmaceutical products, and to methods of treating a psychiatric patient in need of treatment with long acting injectable paliperidone palmitate formulations.

### BACKGROUND OF THE INVENTION

Antipsychotic medications are the mainstay in the treatment of schizophrenia, schizoaffective disorder, and schizophreniform disorders. Antipsychotics were first introduced in the mid-1950s. These typical or first generational drugs are usually effective in controlling the positive symptoms of schizophrenia but are less effective in moderating the negative symptoms or the cognitive impairment associated with the disease. Atypical antipsychotics or second generation drugs, typified by risperidone and olanzapine, were developed in the 1990s, and are generally characterized by effectiveness against both the positive and negative symptoms associated with schizophrenia.

Paliperidone palmitate is the palmitate ester of paliperidone (9-hydroxy-risperidone), a monoaminergic antagonist that exhibits the characteristic dopamine D₂ and serotonin (5-hydroxytryptamine type 2A) antagonism of the second generation, atypical antipsychotic drugs. Paliperidone (9-OH risperidone) is the major active metabolite of risperidone. Extended release (ER) osmotic controlled release oral delivery (OROS) paliperidone, as a tablet formulation, is marketed in the United States (U.S.) for the treatment of schizophrenia and maintenance of effect.

Paliperidone palmitate has been developed as a long-acting, intramuscular (i.m.), injectable aqueous nanosuspension for the treatment of schizophrenia and other related diseases that are normally treated with antipsychotic medications. Because of extreme low water solubility, paliperidone esters such as paliperidone palmitate dissolve slowly after an intramuscular injection before being hydrolyzed to paliperidone and made available in the systemic circulation.

Many patients with mental illnesses achieve symptom stability with available oral antipsychotic medications; however, it is estimated that up to 75% have difficulty adhering to a daily oral treatment regimen, i.e. adherence problems. Problems with adherence often result in worsening of symptoms, suboptimal treatment response, frequent relapses and re-hospitalizations, and an inability to benefit from rehabilitative and psychosocial therapies. Once monthly Paliperidone palmitate injection has been developed to provide sustained plasma concentrations of paliperidone, which may greatly enhance compliance with dosing. Paliperidone palmitate formulated as an aqueous nanosuspension is described in U.S. Pat. Nos. 6,077,843 and 6,555,544, each of which is incorporated herein by reference. In addition, dosing regimens of paliperidone palmitate for treating patients is disclosed in U.S. Pat. Nos. 9,439,906 and 10,143,693, each of which is incorporated herein by reference.

Paliperidone palmitate is an atypical antipsychotic drug administered by intramuscular injection. The original formulation of paliperidone palmitate was a once-monthly antipsychotic and was approved for the treatment of schizophrenia in adults in numerous countries. The acute and sustained efficacy and tolerability profile of once-monthly paliperidone palmitate has been demonstrated in clinical studies totaling more than 3800 patients. Continued treatment with once-monthly paliperidone palmitate in patients who initially responded to it for acute worsening of symptoms resulted in a nearly 4-fold reduction in relapse risk compared with patients randomized to placebo. A later developed three-month formulation offers a substantially longer dosing interval: injections are administered once every three months. This extended dosing interval offers the prospect of fewer opportunities for nonadherence than previously available long acting injectable formulations, thus reducing relapse risk as a result of subtherapeutic plasma concentration and its associated negative consequences in patients with schizophrenia.

Paliperidone is currently available for therapeutic use in 3 formulations: an oral extended-release formulation (INVEGA® Extended Release [ER] tablets; also termed INVEGA® prolonged-release [PR] tablets), and two long-acting injectable (LAI) formulations (paliperidone palmitate one-month injection [INVEGA SUSTENNA® or XEPLION®] and paliperidone palmitate three-month injection [INVEGA TRINZA® or TREVICTA®]). As disclosed herein, another paliperidone palmitate product intended for administration once every six months (paliperidone palmitate six-month injection), with a view to further improving adherence and convenience, is being developed.

Patients who do not regularly take their medications can suffer many consequences, most notably schizophrenia relapse. For oral antipsychotics, medication gaps of as little as one day can double the risk for re-hospitalization. This typically results in worsening of psychiatric comorbidities, loss of employment, interruption of education and impairment of family relationships. The biological consequences include loss of synaptic plasticity of neurons, especially in the frontal lobes. Functionally schizophrenia relapse has been linked to pruning at the level of synaptic neuronal junctions. Overall, this can be radiologically measured by widespread shrinkage of the grey matter of the brain, with accompanying enlargement of the cerebral ventricles. These changes are visible on CT/MRI scanning of the brain. With each successive relapse, further progressive changes to the brain are typically observed. Currently there is no known cure for schizophrenia, and the only proven method to treat the disease is with long-term administration of antipsychotic medications along with social and behavioral interventions. The strongest predictor of schizophrenia relapse is adherence to antipsychotic medications.

A paliperidone palmitate product that is intended to be given once every six months presents a challenge in terms of a patient remembering to come in for treatment at exactly the six-month time point. This is further compounded by the fact that the length of a month varies between 28-31 days. Because an injection is intended to be given by a health care professional, and not self-administered, allowing patients to have flexibility to schedule their visit to the clinic and receive their injection is an important consideration. Most other antipsychotic regimens (oral and LAI) are typically given over a one-month cycle, and patients return to a clinic to either get a refill of their prescription or an injection. A six-month dosing interval presents a unique challenge to ensure compliance.

Patients also at times miss their doses of medication. Consequently, there is a need to re-initiate a dosing regimen for patients who miss their regularly scheduled dose of medication.

Moreover, weight gain is a very common phenomenon in the treatment of patients requiring antipsychotic medication. During long-term treatment with antipsychotic drugs in patients with schizophrenia or schizoaffective or any other psychotic disorder, obesity and other cardiovascular risk factors increase and mostly negatively impact patients long-term morbidity and even mortality. Patients with severe mental disorders also face stigmatization and a reduced quality of life due to treatment side-effects like weight gain, especially young patients in early stages of their illness. Avoiding or stabilizing weight gain can help those patients maintain their social life, reduce stigma, and increase quality-of-life. Any potential for a decrease of body weight or a stabilization of current weight would be a benefit for patients treated with risperidone or paliperidone who are in need of long-term symptom protection.

### SUMMARY OF THE INVENTION

In one embodiment, the present disclosure provides methods for administering paliperidone palmitate to a patient in need thereof who has been administered a first dose of a paliperidone palmitate extended-release injectable suspension, comprising administering a second dose of the paliperidone palmitate extended-release injectable suspension to a deltoid or gluteal muscle of the patient up to two weeks before or three weeks after a time that is six months after administration of the first dose, without an intervening dose of paliperidone palmitate between the first dose and the second dose.

In other embodiments, the disclosure provides re-initiation dosing regimens for patients who miss their regularly scheduled dose of medication, with the regimen depending on the time elapsed from the patient's last dose. For example, the disclosure provides methods for administering paliperidone palmitate to a patient in need thereof who has been administered a first dose of a first paliperidone palmitate extended-release injectable suspension (first suspension), comprising administering in a deltoid muscle of the patient a re-initiation loading dose of a second paliperidone palmitate extended-release injectable suspension (second suspension) at a time that is more than six months and three weeks after administration of the first dose of the first suspension but less than eight months after administration of said first dose of the first suspension; and administering in a deltoid or gluteal muscle of the patient a maintenance dose of the first suspension at a time that is about one month (±7 days) after administering the re-initiation loading dose of the second suspension.

Other re-initiation regimens include administering paliperidone palmitate to a patient in need thereof who has been administered a first dose of a first paliperidone palmitate extended-release injectable suspension (first suspension) wherein such administration involves administering to a deltoid muscle of the patient a first re-initiation loading dose of 156 mg paliperidone palmitate of a second paliperidone palmitate extended-release injectable suspension (second suspension) at a time that is from eight months up to and including eleven months after administration of the first dose of the first suspension; administering in a deltoid muscle of the patient a second re-initiation loading dose of 156 mg paliperidone palmitate of the second suspension on about day 8 (±4 days) after administering the first re-initiation loading dose of the second suspension; and administering in a deltoid or gluteal muscle of the patient from about 1092 mg to about 1560 mg paliperidone palmitate of a maintenance dose of the first suspension about one month (±7 days) after administering the second re-initiation loading dose of the second suspension.

Other re-initiation regimens include administering paliperidone palmitate to a patient in need thereof who has been administered a first dose of a first paliperidone palmitate extended-release injectable suspension (first suspension), wherein such administration involves (1) administering in a deltoid muscle of the patient a first re-initiation loading dose of 234 mg of paliperidone palmitate of a second paliperidone palmitate extended-release injectable suspension (second suspension) at a time that is more than eleven months after administration of the first dose of the first suspension; (2) administering in a deltoid muscle of the patient a second re-initiation loading dose of 156 mg of paliperidone palmitate of the second suspension on about day 8 (±4 days) after administering the first re-initiation loading dose of the second suspension; (3) administering in a deltoid or gluteal muscle of the patient a first re-initiation maintenance dose of 39 mg to about 234 mg of paliperidone palmitate of the second suspension about one month (±7 days) after administering the second re-initiation loading dose; (4) administering in a deltoid or gluteal muscle of the patient a second re-initiation maintenance dose of from about 39 mg to about 234 mg of paliperidone palmitate of the second suspension about one month (±7 days) after administering the first re-initiation maintenance dose of second suspension; (5) administering in a deltoid or gluteal muscle of the patient a third re-initiation maintenance dose of from about 39 mg to about 234 mg of paliperidone palmitate of the second suspension about one month (±7 days) after administering the second re-initiation maintenance dose of the second suspension; and (6) administering in a deltoid or gluteal muscle of the patient from about 1092 mg to about 1560 mg of paliperidone palmitate of a maintenance dose of the first suspension about one month (±7 days) after administering the third re-initiation maintenance dose of the second suspension. Additional re-initiation maintenance doses may be administered before the maintenance dose of the first suspension (e.g. a fourth re-initiation maintenance dose, fifth re-initiation maintenance dose, etc.). In certain embodiments, the re-initiation maintenance doses of paliperidone palmitate are from about 156 to about 234 mg.

The disclosure also provides methods of stabilizing or decreasing body weight of a patient who has been treated with a paliperidone palmitate extended-release injectable suspension at either one-month intervals (PP1M) or three-month intervals (PP3M), comprising administering a last dose of the PP1M or the PP3M and then administering an initial dose of a paliperidone palmitate extended-release injectable suspension having a six month dosing interval (PP6M).

The disclosure further relates to a pharmaceutical product comprising a paliperidone palmitate extended-release injectable suspension having a six month dosing interval (PP6M), wherein the pharmaceutical product is packaged, and wherein the package includes a label that identifies the PP6M as a an approved drug product for the treatment of schizophrenia after a patient has been adequately treated with either a one-month paliperidone palmitate extended-release injectable suspension (PP1M) for at least four months or a three-month paliperidone palmitate extended-release injectable suspension following at least one 3-month injection cycle.

The disclosure is also directed to an approved drug product in a prefilled syringe, wherein the approved drug product comprises an extended release injectable suspension of 1092 mg of paliperidone palmitate in a 3.5 ml volume or 1560 mg of paliperidone palmitate in a 5 ml volume.

The disclosure provides methods for treating schizophrenia in a patient in need thereof, comprising administering an approved drug product comprising PP6M in an amount and manner that is described in a drug product label for the approved drug product and/or in a treatment regimen described herein.

The disclosure also provides methods of selling an approved drug product comprising PP6M, said method comprising selling such drug product, wherein a drug product label for a reference listed drug for such drug product includes instructions for treating a patient with schizophrenia after the patient has been adequately treated with either PP1M for at least four months or PP3M following at least one 3-month injection cycle.

The disclosure further provides methods of offering for sale a drug product comprising PP6M, said method comprising offering for sale such drug product, wherein a drug product label for a reference listed drug for such drug product includes instructions for treating a patient with schizophrenia after the patient has been adequately treated with either PP1M for at least four months or PP3M following at least one 3-month injection cycle.

### BRIEF DESCRIPTION OF THE DRAWINGS

FIG. 1 depicts a flow chart of a double-blind, randomized, active-controlled, parallel-group study of paliperidone palmitate six-month formulation.

FIG. 2 depicts a Kaplan-Meier plot of time to relapse during the double-blind phase up to month 12.

FIG. 3 depicts a Forest plot of estimated percentage (95% CI) of subjects that remained relapse free at month 12.

FIG. 4 depicts median plasma concentration time profiles of paliperidone after administration of PP3M (350 mg eq. or 525 mg eq.) and PP6M (700 mg eq. or 1000 mg eq.) in a double-blind study.

FIG. 5 depicts a comparison of PK plasma concentration and clinical efficacy (median time to relapse) across paliperidone formulations.

FIG. 6 depicts missed dose simulations for when >6 months and 3 weeks and up to 8 months have elapsed since the last steady-state 1000 mg eq. PP6M injection (7 months, and 7.5 months after the last PP6M dose).

FIG. 7 depicts missed dose simulations when between 8 months up to and including 11 months have elapsed since the last 1000 mg eq. PP6M injection (8, 10 and 11 months after the last PP6M dose).

FIG. 8 depicts missed dose simulations for when >11 months have elapsed since the last 1000 mg eq. PP6M injection (12, 15 and 18 months after the last PP6M dose).

FIG. 9 depicts bar graphs showing mean weight change and abnormal weight change from double-blind baseline for patients treated with PP6M.

FIG. 10 depicts a bar graph showing mean weight change of patients of various weight class (normal, overweight and obese) being treated with PP6M.

FIG. 11 depicts a bar graph showing mean weight change of patients of various age groups being treated with PP6M.

FIG. 12 depicts a bar graph showing the effects of paroxetine, carbamazepine and divalproex sodium ER on paliperidone pharmacokinetics.

FIG. 13 depicts a bar graph showing the effects of paliperidone on divalproex sodium ER pharmacokinetics.

FIG. 14 depicts a bar graph showing the effects of intrinsic factors on paliperidone pharmacokinetics.

FIG. 15 depicts a Kaplan-Meier plot of cumulative proportion of patients with relapse over time, including 95% pointwise confidence bands.

### DETAILED DESCRIPTION

The presently disclosed inventive subject matter may be understood more readily by reference to the following detailed description taken in connection with the accompanying examples, which form a part of this disclosure. It is to be understood that these inventions are not limited to the specific formulations, methods, or parameters described and/or shown herein, and that the terminology used herein is for the purpose of describing particular embodiments by way of example only and is not intended to be limiting of the claimed inventions.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function.

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the invention that are, for brevity, described in the context of a single embodiment, may also be provided separately or in any sub-combination.

Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

As used herein, “PP1M” refers to a paliperidone palmitate extended-release injectable suspension or other type of formulation having an amount of paliperidone palmitate suitable for a dosing interval of about one-month, e.g. an once-a-month paliperidone palmitate extended-release injectable suspension. For example, PP1M can refer to a paliperidone palmitate extended-release injectable suspension having an amount of paliperidone palmitate suitable for a dosing interval of about one-month. A commercially available example includes INVEGA SUSTENNA® or XEPLION®. See also U.S. Pat. No. 9,439,906 incorporated herein by reference. In one embodiment, PP1M is administered by intramuscular injection. In one embodiment, PP1M is administered by intramuscular injection into a deltoid muscle or a gluteal muscle. In one embodiment, PP1M is administered by intramuscular injection into a deltoid muscle. In one embodiment, PP1M is administered by intramuscular injection into a gluteal muscle.

As used herein, “PP3M” refers to a paliperidone palmitate extended-release injectable suspension or other type of formulation having an amount of paliperidone palmitate suitable for a dosing interval of about three-months, e.g., an once every-three-month paliperidone palmitate extended-release injectable suspension. For example, PP3M can refer to a paliperidone palmitate extended-release injectable suspension having an amount of paliperidone palmitate suitable for a dosing interval of about three-months. A commercially available example includes INVEGA TRINZA® or TREVICTA®. See also U.S. Pat. No. 10,143,693 incorporated herein by reference. In one embodiment, PP3M is administered by intramuscular injection. In one embodiment, PP3M is administered by intramuscular injection into a deltoid muscle or a gluteal muscle. In one embodiment, PP3M is administered by intramuscular injection into a deltoid muscle. In one embodiment, PP3M is administered by intramuscular injection into a gluteal muscle.

As used herein, “PP6M” refers to a paliperidone palmitate extended-release injectable suspension or other type of formulation having an amount of paliperidone palmitate suitable for a dosing interval of about six-months, e.g., an once every-six-month paliperidone palmitate extended-release injectable suspension. For example, PP6M can refer to a paliperidone palmitate extended-release injectable suspension having an amount of paliperidone palmitate suitable for a dosing interval of about six-months. A commercially available example includes INVEGA HAFYERA™ or BYANNLI®.

Paliperidone is effective for the treatment of psychosis and has been used to treat schizophrenia and schizoaffective disorders. Accordingly, PP6M is suitable for the treatment of psychotic disorders including but not limited to schizophrenia and/or schizoaffective disorder or bipolar disorder.

PP6M is typically administered to patients who have been adequately treated with PP1M (e.g. INVEGA SUSTENNA®) for several months, and, in certain embodiments, for at least four months, with PP1M doses of about 156 mg or about 234 mg paliperidone palmitate. It is further preferred that the last two doses of PP1M are at the same dosage strength before starting PP6M. Alternatively, PP6M is administered to patients who have been adequately treated with PP3M (e.g. INVEGA TRINZA®) for at least one three-month cycle, with PP3M doses of about 546 mg or about 819 mg paliperidone palmitate.

PP6M will typically be provided with a dose in the range of from about 1000 mg to about 1600 mg of paliperidone palmitate to provide a sustained therapeutic concentration of paliperidone over the six-month dosing interval. Preferably, the PP6M is provided in dose strengths of about 1092 mg or about 1560 mg paliperidone palmitate. The drug product hydrolyzes to the active moiety, paliperidone, resulting in dose strengths of about 700 mg eq. or 1000 mg eq. of paliperidone, respectively.

PP6M is preferably provided in a prefilled syringe (cyclic-olefin-copolymer) prefilled with either 700 mg eq. (3.5 mL) or 1000 mg eq. (5.0 mL) paliperidone (as 1092 mg or 1560 mg paliperidone palmitate, respectively) with a plunger stopper, a plunger rod, and tip cap (bromobutyl rubber), a backstop, and a needle, preferably a thin walled 20 gauge (G), 1½-inch safety needle. In some embodiments, the prefilled syringe and the safety needle, e.g., 20 gauge (G), 1½-inch needle, are provided in a kit.

PP6M is intended for intramuscular use. It is not recommended to administer by any other route. Care should be taken to avoid inadvertent injection into a blood vessel. Doses are preferably administered in a single injection; for example, divided injections could change the release profile. It is otherwise preferred that injections be administered slowly, deep into the muscle of the patient, in particular, a deltoid or a gluteal muscle. Typically, PP6M is administered to a gluteal muscle given the volume of the injection. In one embodiment, PP6M is administered by intramuscular injection. In one embodiment, PP6M is administered by intramuscular injection into a gluteal muscle.

PP6M desirably is administered to an adult patient, i.e., 18 years or older. However, because elderly patients are more likely to have decreased renal function, PP6M is not recommended to be used in elderly patients with mild, moderate or severe renal impairment. In certain aspects, the patient is checked for renal impairment and only dosed if no renal impairment is determined. In other aspects, PP6M is not recommended for use in patients with mild, moderate, or severe renal impairment (creatinine clearance <90 mL/min) because necessary dosage adjustment is not possible.

It should be understood that references herein to methods of treatment using one or more compounds or formulations thereof (e.g. a paliperidone palmitate extended-release injectable suspension) should also be interpreted as references to:
one or more compounds or formulations thereof (e.g. a paliperidone palmitate extended-release injectable suspension) for use in methods of treatment; and/or
the use of one or more compounds or formulations thereof (e.g. a paliperidone palmitate extended-release injectable suspension) in the manufacture of a medicament for treating a pathological condition.

### Intramuscular Injection

Typically, regardless of the patient's weight, PP6M is administered intramuscularly using a thin walled syringe, for example, a 20 gauge (G), 1½-inch needle in a deltoid muscle or a gluteal muscle. To the extent administered to a deltoid muscle, paliperidone palmitate is typically administered into the center of a deltoid muscle, preferably alternating between the two deltoid muscles per single injection (i.e. the opposite deltoid muscle is used at the next scheduled dosing interval). For PP6M, gluteal intramuscular administration is preferred. For example, PP6M may be administered into the upper-outer quadrant of a gluteal muscle. It is also preferred that gluteal injections should be alternated between the two gluteal muscles per single injection (i.e. the opposite gluteal muscle is used at the next scheduled dosing interval). Similarly, PP1M and PP3M are intended for intramuscular use, and may be administered consistent with the prescribing information for those products. For example, such administration may be to a deltoid or a gluteal muscle. Regardless of the selected muscle, the selected injection site is cleansed, i.e., wiped with alcohol and allowed to dry prior to injection. In certain aspect, the site of injection is not touched, fanned, or blown on after cleansing.

### Incomplete Administration

PP6M is typically a highly concentrated product. As a result, an important consideration is to ensure complete suspension/resuspension of the product before administration. To avoid an incomplete administration, the syringe is shaken and/or mechanically agitated to obtain a uniform dispersion of the suspension. For example, the syringe is preferably shaken fast, preferably very fast, with the syringe tip cap pointing up for at least 15 seconds. A brief rest may be taken, and then the syringe may be, and preferably is, shaken again for another 15 seconds. In certain aspects, the syringe is shaken in short, up and down motions. In other aspects, the syringe is shaken using a loose wrist of the person holding the syringe.

The shaken syringe is then inspected to determine if it is acceptable for injection. The syringe may be inspected for particulate matter and discoloration prior to injection. In some embodiments, a syringe that is acceptable for injection appears uniform, thick, and milky white. In other embodiments, a syringe that is unacceptable for injection has one or more of solid product on the side and/or top of the syringe, an uneven mix, or thin liquid. If the syringe is unacceptable for injection, it is shaken syringe, with the syringe tip cap pointing up very fast, for at least about 15 seconds, a brief rest is taken, and then the syringe is shaken again for about 15 seconds.

The injection is then preferably done immediately or within 5 minutes of the last shaking to ensure resuspension and that the needle does not get clogged during injection. If more than about 5 minutes pass before the injection occurs, the syringe may be shaken very fast, with the top cap pointing up again, for at least about 30 seconds. Desirably, this resuspends the solid after shaking.

In some embodiments, the carton is shipped and stored in a horizontal orientation. This improves the ability to resuspend this highly concentrated product.

Due to the slow release characteristics of PP6M, the product is not intended to be used in patients who are immediately transitioning from oral to LAI antipsychotic therapy. Rather, PP6M is intended to be used in patients who are adequately treated with either PP1M or PP3M at the time of initiation of PP6M. The determination of adequately treated is typically up to the judgment of the prescribing clinician. Typically, PP6M dosing is initiated: A) one month after being adequately treated with PP1M (e.g. INVEGA SUSTENNA®) for at least four months; or B) three months after a PP3M (e.g. INVEGA TRINZA®) dose has been established as adequate treatment. PP6M may be administered one month (±7 days) after a last PP1M injection, or three months (±14 days) after a last PP3M injection.

Following the initial PP6M injection, PP6M should be administered every six months. If needed, dose adjustment can be made every six months in increments within the range of 1092 mg to 1560 mg paliperidone palmitate based on individual patient tolerability and/or efficacy. Typically, the dosage is adjusted to about 1092 mg or to about 1560 mg paliperidone palmitate. Due to the long-acting nature of PP6M, the patient's response to an adjusted dose may not be apparent for several months.

### Dosing Window

As noted herein, nonadherence is a major issue in the treatment of psychiatric patients, especially those with schizophrenia, who often abruptly discontinue medication without consulting their practitioner or caregiver. Lack of adherence has been identified as the strongest predictor of relapse, which typically results in worsening of psychiatric comorbidities, loss of employment, interruption of education and impairment of family relationships. For oral antipsychotics, medication gaps of as little as one day can double the risk for re-hospitalization. Long acting injectable (LAI) antipsychotics were developed to address this problem and to ensure timely interventions for non-adherent patients to prevent relapse and hospitalization.

The commonly encountered difficulty in clinical practice, however, is that subjects need to return to the clinic on a specific date after receiving their previous maintenance injection of a LAI antipsychotic. Having a window in which injections could be given would give greater flexibility to prescribers, patients, and caregivers. This window will often be prescribed by a medical professional and/or set by a regulatory agency.

Previously, a dosing window of ±1 week around the target injection date (scheduled injection date based on dosing interval) was established for PP1M. For PP3M, this window was expanded to ±2 weeks around the target injection date. It has now been found that a dosing window of up to 2 weeks earlier and up to 3 weeks after the target injection date for PP6M may be used, offering further dosing flexibility.

Dosing recommendations have historically been aimed at paliperidone plasma concentrations above a threshold of 7.5 ng/mL. This threshold has been associated with a central Dopamine type 2 (D2) receptor occupancy of 60% while receptor occupancy in the range of 60%-80% is considered necessary for a satisfactory antipsychotic response. As reflected in the example section (see Example 7), simulations were conducted to evaluate the relationship between median time to relapse and the point at which the median paliperidone concentration goes under 7.5 ng/mL. An apparent delay lasting from several weeks to several months was observed between the time point when median plasma paliperidone concentration decreased to 7.5 ng/mL and the median time to relapse, i.e., the time point when half of the subjects had experienced relapse, while the other half of the subjects either relapsed later or did not relapse during the study. Thus, it appears that the therapeutic effect is more prolonged than the expected effect based on the 7.5 ng/mL threshold, and the relapse protection window may be extended farther in the positive direction.

In one embodiment, a dosing window of up to 2 weeks earlier and up to 3 weeks after the target injection date for PP6M (i.e. the scheduled six-month time point) is used. Accordingly, the disclosure includes methods for administering paliperidone palmitate to a patient in need thereof who has been administered a first dose of a paliperidone palmitate extended-release injectable suspension, comprising administering a second dose of the paliperidone palmitate extended-release injectable suspension to a deltoid or gluteal muscle, preferably a gluteal muscle, of the patient up to two weeks before or three weeks after a time that is six months after administration of the first dose, without an intervening dose of paliperidone palmitate between the first dose and the second dose. It should be recognized that the first dose and the second dose are doses within the sequence of the described methods, but do not necessarily refer to the very first (initial) or second dose administered to the patient.

Unless otherwise indicated, as used herein, a “month” refers to a Gregorian calendar month and may vary from as little as 28 days (e.g., February) to 31 days (e.g., October), e.g., 28, 29, 30, or 31 days. The six-month time point reflects six consecutive calendar months. As indicated herein, certain testing reflected in the examples, including simulations, were based on 30 days being used for a month. A “week” refers to seven days.

In some embodiments, other dosing windows may be considered. For example, a dosing window of up to one week earlier and up to two weeks after, or up to one week earlier and up to three weeks after, or up to two weeks earlier and up to two weeks after, the target injection date for PP6M. In still other embodiments, the second dose is administered up to four weeks, or up to five weeks after a time that is six months after administration of the first dose as part of a dosing window. Any combination of these time periods before and after a time that is six months after administration of the first dose may be utilized. In certain embodiments, the dosing window is applied to patients that have reached a steady-state paliperidone plasma concentration.

The first dose and second dose, independently, are typically from about 1000 mg to about 1600 mg of paliperidone palmitate. In particular, the first dose and second dose, independently, are about 1092 mg or about 1560 mg paliperidone palmitate. In other embodiments, the first dose and the second dose are each about 1092 mg paliperidone palmitate. In another embodiment, the first dose and second dose are each about 1560 mg paliperidone palmitate.

Typically, the plasma concentration of paliperidone in the patient is about 5 to about 50 ng/mL at the time of the second dose, or about 10 to about 40 ng/mL at the time of the second dose. For example, when the first dose is 1092 mg paliperidone palmitate, the plasma concentration of paliperidone in the patient is about 5 to about 30 ng/mL, or about 10 to about 25 ng/mL, at the time of the second dose. When the first dose is 1560 mg paliperidone palmitate, the plasma concentration of paliperidone in the patient is about 9 to about 40 ng/mL, or about 20 to about 30 ng/mL, at the time of the second dose. In this context, “at the time of the second dose” refers to concentration levels immediately preceding the second dose, typically representing C_{trough} values.

In other aspects, the plasma concentration of paliperidone in the patient achieves a peak of about 10 to about 150 ng/mL after administration of the second dose, or about 35 ng/mL to about 125 ng/mL after administration of the second dose. For example, when the first dose is 1092 mg paliperidone palmitate, the plasma concentration of paliperidone in the patient achieves a peak of about 10 to about 125 ng/mL, or about 50 to about 90 ng/mL, after administration of the second dose. When the first dose is 1560 mg paliperidone palmitate, the plasma concentration of paliperidone in the patient achieves a peak of about 35 to about 145 ng/mL, or about 70 to about 110 ng/mL, after administration of the second dose.

In certain embodiments, PP3M and PP6M can have the same formulation. In such an embodiment, the pharmacokinetic properties of PP6M will be similar to PP3M, but such a PP6M would be expected to have higher peaks and lower troughs given the higher amount of drug and longer frequency of administration. As shown in Example 6, the absolute drug plasma concentrations were lower for PP6M at its target interval vs. PP3M at its target interval. Given that PP3M had established a dosing window of ±2 weeks around the target injection date, expanding the dosing window in the positive direction for a formulation that results in lower drug plasma concentrations at the target interval point would not have been suggested. But, as reflected herein, it was discovered that the therapeutic effect is more prolonged than the expected effect based on pharmacokinetic data, thus allowing the dosing window to extend farther in the positive direction.

In other embodiments, mean trough concentrations (C_{trough}) at the end of the dosing interval are about 20-25% lower for PP6M as compared to corresponding doses of PP3M. The Cₘₐₓ was higher (1.4 to 1.5-fold) for PP6M as compared to corresponding doses of PP3M. Inter-subject variability in paliperidone PK parameters for PP6M ranged from about 42 to about 48% for AUC_{6 months} and ranged from about 56 to about 103% for Cₘₐₓ.

In further embodiments, following gluteal injection(s) of PP6M at doses of 1,092 or 1,560 mg plasma concentrations of paliperidone rise to reach maximum concentrations at a median Tₘₐₓ of about 29 to about 32 days. The release profile and dosing regimen of PP6M results in sustained concentrations over about 6 months. The total and peak dose-normalized exposures of paliperidone following PP6M administration were comparable between 1,092 mg and 1,560 mg dose levels. The median steady-state peak:trough ratio for an PP6M dose is about 3.1 and about 3.0 following gluteal administration of 1,092 and 1,560 mg respectively.

In yet other embodiments, the concentration of paliperidone remaining in the circulation about 18 months after dosing of 1,560 mg of PP6M is estimated to be about 18% of the average steady-state levels.

### Missed Doses

Patients who receive LAI antipsychotics routinely return to their health care provider to receive injections of their medication. The timing of their dose is typically carefully prescribed. As noted herein, for any given antipsychotic an optimal dosing cycle is recommended, along with a dosing window (±) in which they can receive their medication without any untoward side effects or loss of efficacy. In the present disclosure, following an initial dose of PP6M, PP6M should be administered every six months. Missed doses of PP6M should be avoided, although injections given within the prescribed dosing window would not be considered a missed dose. If needed, dose adjustment can be made every six months between the dose levels of 1092 mg to 1560 mg paliperidone palmitate based on individual patient tolerability and/or efficacy.

However, despite this, it is a frequent occurrence for schizophrenia patients to become noncompliant at some point during their illness. Therefore, based on population pharmacokinetic simulations, guidelines are provided in the event of missed doses of PP6M beyond the dosing-window.

The present disclosure provides a mechanism by which patients can resume treatment with PP6M in case they become fully or partially non-adherent. Since dosing of PP6M is dependent on a patient first being stabilized on PP1M/PP3M, this would reduce the necessity of patients having to start de-novo. In addition, because it was discovered that the therapeutic effect is more prolonged than the expected effect based on pharmacokinetic data, patients that had at least one PP6M injection are expected to be relapse-free for a longer period of time. This provides a positive effect of PP6M on preventing relapse, even in situations of non-adherence.

The disclosure provides re-initiation dosing regimens for patients who miss their regularly scheduled dose of medication, i.e., are outside the prescribed dosing window, with the regimen depending on the time elapsed from the patient's last dose. In some embodiments, the missed dose is over six months and three weeks, but less than seven to nine months, e.g. less than eight months, after the last injection.

For example, to the extent the dosing window is up to 2 weeks earlier and up to 3 weeks after the target injection date, the disclosure provides a method for administering paliperidone palmitate to a patient in need thereof who has been administered a first dose of a first paliperidone palmitate extended-release injectable suspension (first suspension), comprising administering in a deltoid muscle of the patient a re-initiation loading dose of a second paliperidone palmitate extended-release injectable suspension (second suspension) at a time that is more than six months and three weeks after administration of the first dose of the first suspension but less than seven to nine months, e.g. less than eight months, after administration of said first dose of the first suspension; and administering in a deltoid or gluteal muscle of the patient a maintenance dose of the first suspension at a time that is about one month (±7 days) after administering the re-initiation loading dose of the second suspension. In one embodiment, the re-initiation loading dose of the second suspension and the maintenance dose of the first suspension are selected based on the first dose of the first suspension as shown below in Table 1, with the administration of the first suspension preferably in a gluteal muscle of the patient:

TABLE 1

| | Re-initiation | Maintenance |
|---|---|---|
| First dose of | loading dose of | dose of first |
| first suspension | second suspension | suspension |
| 1092 mg | 156 mg | 1092 mg |
| 1560 mg | 234 mg | 1560 mg |

To the extent a different dosing window is prescribed, the same re-initiation dosing regimen noted above may be implemented but adjusted based on the outer dosing window parameter. For example, if the dosing window was up to 1 week earlier and up to 2 weeks after the target injection date, the re-initiation loading dose of the second suspension would be administered at a time that is more than six months and two weeks after administration of the first dose of the first suspension but less than seven to nine months, e.g. less than eight months, after administration of said first dose of the first suspension.

Other re-initiation regimens are based on a missed dose of seven to nine months and up to and including ten to fourteen months after the last injection. For example, the disclosure includes administering paliperidone palmitate to a patient in need thereof who has been administered a first dose of a first paliperidone palmitate extended-release injectable suspension (first suspension), comprising administering to a deltoid muscle of the patient a first re-initiation loading dose of 156 mg paliperidone palmitate of a second paliperidone palmitate extended-release injectable suspension (second suspension) at a time that is from seven to nine months, e.g. from eight months, up to and including ten to fourteen months, e.g. up to and including eleven months, after administration of the first dose of the first suspension; administering in a deltoid muscle of the patient a second re-initiation loading dose of 156 mg paliperidone palmitate of the second suspension on about day 8 (±4 days) after administering the first re-initiation loading dose of the second suspension; and administering in a deltoid or gluteal muscle of the patient from about 1092 mg to about 1560 mg paliperidone palmitate of a maintenance dose of the first suspension about one month (±7 days) after administering the second re-initiation loading dose of the second suspension. In one embodiment, the first dose of the first suspension is about 1092 mg paliperidone palmitate and the maintenance dose of the first suspension is about 1092 mg paliperidone palmitate. In another embodiment, the first dose of the first suspension is about 1560 mg paliperidone palmitate and the maintenance dose of the first suspension is about 1560 mg paliperidone palmitate. In preferred embodiments, the administration of the first suspension is in a gluteal muscle of the patient.

Other re-initiation regimens are based on a missed dose of more than ten to fourteen months after the last injection. For example, the disclosure includes administering paliperidone palmitate to a patient in need thereof who has been administered a first dose of a first paliperidone palmitate extended-release injectable suspension (first suspension), comprising (1) administering in a deltoid muscle of the patient a first re-initiation loading dose of 234 mg paliperidone palmitate of a second paliperidone palmitate extended-release injectable suspension (second suspension) at a time that is more than ten to fourteen months, e.g. more than eleven months, after administration of the first dose of the first suspension; (2) administering in a deltoid muscle of the patient a second re-initiation loading dose of 156 mg paliperidone palmitate of the second suspension on about day 8 (±4 days) after administering the first re-initiation loading dose of the second suspension; (3) administering in a deltoid or gluteal muscle of the patient a first re-initiation maintenance dose of 39 mg to about 234 mg paliperidone palmitate of the second suspension about one month (±7 days) after administering the second re-initiation loading dose; (4) administering in a deltoid or gluteal muscle of the patient a second re-initiation maintenance dose of from about 39 mg to about 234 mg paliperidone palmitate of the second suspension about one month (±7 days) after administering the first re-initiation maintenance dose of the second suspension; (5) administering in a deltoid or gluteal muscle of the patient a third re-initiation maintenance dose of from about 39 mg to about 234 mg paliperidone palmitate of the second suspension about one month (±7 days) after administering the second re-initiation maintenance dose of the second suspension; and (6) administering in a deltoid or gluteal muscle of the patient from about 1092 mg to about 1560 mg paliperidone palmitate of a maintenance dose of the first suspension about one month (±7 days) after administering the third re-initiation maintenance dose of the second suspension. Preferably, the first suspension is administered in a gluteal muscle. In one embodiment, the first dose of the first suspension is about 1092 mg paliperidone palmitate and the maintenance dose of the first suspension is about 1092 mg paliperidone palmitate. In another embodiment, the first dose of the first suspension is about 1560 mg paliperidone palmitate and the maintenance dose of the first suspension is about 1560 mg paliperidone palmitate. In other embodiments, the second and third re-initiation maintenance doses of second suspension are the same. Additional re-initiation maintenance doses may be administered in one-month (±7 days) intervals before the maintenance dose of the first suspension (e.g. a fourth re-initiation maintenance dose, a fifth re-initiation maintenance dose, etc.). In certain embodiments, the re-initiation maintenance doses of paliperidone palmitate are from about 156 to about 234 mg. With respect to any of the re-initiation regimens, following the maintenance dose of the first suspension, the first suspension is typically administered in six-month intervals as noted herein.

As noted herein, the first suspension and second suspension are typically administered intramuscularly. Although certain exemplary embodiments are described herein, it should be noted that the first suspension and second suspension may be administered in a deltoid muscle or a gluteal muscle, or in another muscle otherwise directed by a healthcare professional. For example, in certain embodiments, a re-initiation loading dose of the second suspension is administered in a deltoid muscle, and a re-initiation maintenance dose of the second suspension is administered in a deltoid or gluteal muscle of the patient, but other muscles may be used depending on the patient, the suspension, and/or the judgment of a healthcare professional.

In particular embodiments, the first suspension is PP6M and the second suspension is PP1M. Exemplary re-initiation regimens based on PP1M and PP6M are further disclosed in Example 8. A goal of a re-initiation regimen is to achieve a quick return to paliperidone plasma concentrations as before the missed dose without creating an overshoot due to the applied re-initiation regimen. It should be recognized that the methods do not contemplate intervening doses of paliperidone palmitate between the enumerated doses of the missed dosing regimens described herein, e.g., between the first dose and the re-initiation loading dose.

Another aspect of the present disclosure is an observed effect for longer acting paliperidone palmitate treatments on stabilizing or decreasing weight in a patient population for which most treatments cause weight gain. In particular, it has been found that transitioning patients who have been adequately treated with PP1M or PP3M to PP6M can reduce, stop, or potentially partially reverse a paliperidone-induced weight gain while maintaining good pharmacological efficacy and maintaining relapse prevention.

As a result, the present disclosure fulfills an unmet medical need for overweight patients treated with risperidone or paliperidone who are in need of long-term symptom protection and therefore antipsychotic treatment but for whom a continuous weight increase is not acceptable. Increasing weight during long-term treatment has metabolic effects which increase risk factors for higher morbidity and mortality (e.g. due to cardiovascular disease). In addition, increasing weight can impact patients mobility and functionality and reduce quality of life significantly. The PP6M formulation and regimen disclosed herein allows a weight neutral or weight reducing treatment for patients while providing the same efficacy compared to other paliperidone or paliperidone palmitate formulations (e.g. PP1M or PP3M).

In one embodiment, the disclosure provides methods of stabilizing or decreasing body weight of a patient who has been treated with a paliperidone palmitate extended-release injectable suspension at either one-month intervals (PP1M) or three-month intervals (PP3M), comprising administering a last dose of the PP1M or the PP3M and then administering an initial dose of a paliperidone palmitate extended-release injectable suspension having a six month dosing interval (PP6M). In certain embodiments, the patient has been treated with the PP1M for at least four months, at least five months, or at least six months. In other embodiments, the patient has been treated with the PP3M for at least one 3-month interval, at least two 3-month intervals, or at least three 3-month intervals.

In embodiments where the patient has been treated with PP1M, the initial dose of PP6M is administered about one month (±7 days) after the last dose of the PP1M is administered. Typically, when the last dose of the PP1M is about 156 mg paliperidone palmitate, the initial dose of PP6M is about 1092 mg paliperidone palmitate. In other embodiments, when the last dose of the PP1M is about 234 mg paliperidone palmitate, the initial dose of PP6M is about 1560 mg paliperidone palmitate.

In embodiments where the patient has been treated with PP3M, the initial dose of PP6M is administered about three months (±14 days) after the last dose of the PP1M is administered. Typically, when the last dose of the PP3M is about 546 mg paliperidone palmitate, the initial dose of PP6M is about 1092 mg paliperidone palmitate. In other embodiments, when the last dose of the PP3M is about 819 mg paliperidone palmitate, the initial dose of PP6M is about 1560 mg paliperidone palmitate.

Following the initial dose of the PP6M, the PP6M is administered in six-month intervals as noted herein.

Further analysis of the data showed particular benefits in overweight patients (body mass index (BMI) of about 25 and less than about 30) and younger patients (about 18 to about 25 years old).

In certain embodiments, at the time of the last dose of PP1M or PP3M, the patient has a body mass index (BMI) of about 25 to less than about 30.

In other embodiments, at the time of the last dose of PP1M or PP3M, the patient has an age of about 18 years old to about 25 years old.

Typically, weight stabilization refers to a BMI change of about −1 to about +1, or from about −0.5 to about +0.5, from the timepoint of the transition to PP6M (from the time of the initial dose of PP6M). Preferably the BMI change is about zero. With respect to body weight decrease, a negative weight change from the timepoint of the transition to PP6M can be seen as a weight decrease. Such stabilization or weight decrease may occur within about twelve months from the timepoint of the transition to PP6M.

In other aspects, the patient's body weight is assessed or determined at the time of the last dose of the PP1M or PP3M, at the time of the initial dose of PP6M, at subsequent time points following the transition to PP6M, or a combination thereof.

### Paliperidone Palmitate Formulations

Paliperidone esters are antipsychotic agents belonging to the chemical class of benzisoxazole derivatives, which contains a racemic mixture of (+)- and (−)-paliperidone, which are described in U.S. Pat. No. 5,254,556 (incorporated herein by reference). The chemical name for paliperidone palmitate is (±)-3-[2-[4-(6-fluoro-1,2-benzisoxazol-3-yl)-1-piperidinyl]ethyl]-6,7,8,9-tetrahydro-2-methyl-4-oxo-4H-pyrido[1,2-c]pyrimidin-9-yl hexadecanoate. The structural formula is:

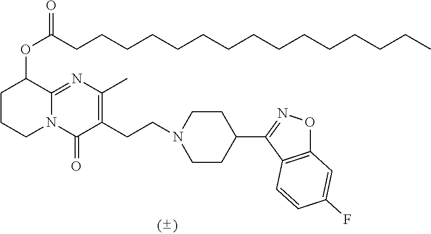

In some embodiments, PP6M is a white to off-white sterile aqueous extended-release suspension for gluteal intramuscular injection in dose strengths of 1,092 mg/3.5 mL and 1,560 mg/5 mL paliperidone palmitate. In certain aspects, the mean (SD) duration of exposure of PP6M is about 329.8 (86.97) days.

Paliperidone esters may be formulated with pharmaceutical excipients into injectable dosage forms as described in U.S. Pat. Nos. 5,254,556 and 6,077,843 both of which are incorporated herein by reference. Injectable formulations may be formulated in aqueous carriers.

As described in U.S. Pat. No. 9,439,906, incorporated herein by reference, a one-month aqueous formulation is a nano particle suspension wherein the nano particles have average sizes of less than about 2,000 nm to about 100 nm. For example, the nano particles have an average particle size (d50) of from about 1,600 nm to about 400 nm, or from about 1,400 nm to about 900 nm. The d90 is less than about 5,000 nm, or less than about 4,400 nm. The d10 is from about 300 nm to about 600 nm. As used herein, d10: the portion of particles with diameters smaller than this value is 10%; d50: the portion of particles with diameters smaller than this value are 50%; d90: the portion of particles with diameters smaller than this value is 90%; when measured by art-known conventional techniques, such as sedimentation field flow fractionation, photon correlation spectroscopy or disk centrifugation.

In certain embodiments, a three-month (PP3M) formulation has average particle sizes of less than about 20 μm to about 1 μm. In other embodiments, the particles have an average particle size (d50) of from about 5 μm to about 15 μm; from about 3 μm to about 10 μm; or from about 5 μm to about 9 μm. The d90 is about 50 μm; from about 10 μm to about 30 μm; or from about 10 μm to about 20 μm. The d10 is from about 1 μm to about 10 μm, or from about 1 μm to about 5 μm.

In certain embodiments, a six-month (PP6M) formulation has average particle sizes of less than about 30 μm to about 1 μm; or about 20 μm to about 1 μm. In other embodiments, the particles have an average particle size (d50) of from about 3 μm to about 25 μm; from about 5 μm to about 15 μm; from about 3 μm to about 10 μm; or from about 5 μm to about 9 μm. The d90 is 60 μm; or about 50 μm; from about 10 μm to about 30 μm; or from about 10 μm to about 20 μm. The d10 is from about 1 μm to about 15 μm; from about 1 μm to about 10 μm; or from about 1 μm to about 5 μm.

Suitable aqueous nanoparticle formulations are described in U.S. Pat. No. 6,555,544 which is incorporated herein by reference. In some embodiments, the formulation comprises micro particles, a surfactant, a suspending agent, and optionally one or more additional ingredients selected from the group consisting of preservatives, buffers and an isotonizing agent.

Useful surface modifiers for paliperidone palmitate formulations are believed to include those that physically adhere to the surface of the active agent but do not chemically bond thereto. Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants. Representative examples of excipients include gelatin, casein, lecithin (phosphatides), gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available TWEENS™, polyethylene glycols, polyoxyethylene stearates, colloidal silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminate silicate, triethanolamine, polyvinyl alcohol (PVA), poloxamers, tyloxapol and polyvinylpyrrolidone (PVP). Most of these excipients are described in detail in the Handbook of Pharmaceutical Excipients, published jointly by the American Pharmaceutical Association and The Pharmaceutical Society of Great Britain, the Pharmaceutical Press, 1986. The surface modifiers are commercially available and/or can be prepared by techniques known in the art. Two or more surface modifiers can be used in combination.

Particularly preferred surface modifiers include polyvinylpyrrolidone; tyloxapol; poloxamers, such as PLURONIC™ F68, F108 and F127 which are block copolymers of ethylene oxide and propylene oxide available from BASF; poloxamines, such as TETRONIC™ 908 (T908) which is a tetrafunctional block copolymer derived from sequential addition of ethylene oxide and propylene oxide to ethylenediamine available from BASF; dextran; lecithin; Aerosol OT™ (AOT) which is a dioctyl ester of sodium sulfosuccinic acid available from Cytec Industries; DUPONOL™ P which is a sodium lauryl sulfate available from DuPont; TRITON™ X-200 which is an alkyl aryl polyether sulfonate available from Rohm and Haas; TWEEN™ 20, 40, 60 and 80 which are polyoxyethylene sorbitan fatty acid esters available from ICI Specialty Chemicals; SPAN™ 20, 40, 60 and 80 which are sorbitan esters of fatty acids; ARLACEL™ 20, 40, 60 and 80 which are sorbitan esters of fatty acids available from Hercules, Inc.; CARBOWAX™ 3550 and 934 which are polyethylene glycols available from Union Carbide; CRODESTA™ F110 which is a mixture of sucrose stearate and sucrose distearate available from Croda Inc.; CRODESTA™ SL-40 which is available from Croda, Inc.; hexyldecyl trimethyl ammonium chloride (CTAC); bovine serum albumin and SA90HCO which is C₁₈H₁₇CH₂(CON(CH₃)CH₂(CHOH)₄CH₂OH)₂. The surface modifiers which have been found to be particularly useful include tyloxapol and a poloxamer, preferably, Pluronic™ F108 and Pluronic™ F68.

Pluronic™ F108 corresponds to poloxamer 338 and is the polyoxyethylene, polyoxypropylene block copolymer that conforms generally to the formula HO[CH₂CH₂O]ₓ[CH(CH₃)CH₂O]_{y}[CH₂CH₂O]_{z}H in which the average values of x, y and z are respectively 128, 54 and 128. Other commercial names of poloxamer 338 are Hodag NONIONIC™ 1108-F available from Hodag, and SYNPERONIC™ PE/F108 available from ICI Americas.

The optimal relative amount of paliperidone palmitate and the surface modifier depends on various parameters. The optimal amount of the surface modifier can depend, for example, upon the particular surface modifier selected, the critical micelle concentration of the surface modifier if it forms micelles, the surface area of the antipsychotic agent, etc. The specific surface modifier preferably is present in an amount of about 0.1 to about 1 mg per square meter surface area of the paliperidone palmitate. It is preferred in the case of paliperidone palmitate (9-hydroxyrisperidone palmitate) to use PLURONIC™ F108 as a surface modifier, a relative amount (w/w) of both ingredients of approximately 6:1 is preferred.

The particles of this invention can be prepared by a method comprising the steps of dispersing paliperidone palmitate in a liquid dispersion medium and applying mechanical means in the presence of grinding media to reduce the particle size of the antipsychotic agent to an effective average particle size. The particles can be reduced in size in the presence of a surface modifier. Alternatively, the particles can be contacted with a surface modifier after attrition.

A general procedure for preparing the particles described herein includes (a) obtaining paliperidone palmitate; (b) adding the paliperidone palmitate to a liquid medium to form a premix; and (c) subjecting the premix to mechanical means in the presence of a grinding medium to reduce the effective average particle size.

The paliperidone palmitate may be prepared using techniques known in the art. It is preferred that the particle size of the paliperidone palmitate be less than about 100 μm as determined by sieve analysis. If the particle size of the paliperidone palmitate is greater than about 100 μm, then it is preferred that the particles of paliperidone palmitate be reduced in size to less than 100 μm.

The paliperidone palmitate can then be added to a liquid medium in which it is essentially insoluble to form a premix. The concentration of paliperidone palmitate in the liquid medium (weight by weight percentage) can vary widely and depends on the selected surface modifier and other factors. Suitable concentrations of paliperidone palmitate in compositions vary from about 0.1% to about 60%, preferably is from about 0.5% to about 30%, and more preferably, is approximately 7% (w/v). For PP1M, it is currently preferred to use a concentration of about 100 mg eq. of paliperidone per mL or about 156 mg of paliperidone palmitate per mL. For PP3M, it is preferred to use a concentration of about 200 mg eq. of paliperidone per mL or about 312 mg of paliperidone palmitate per mL. For PP6M, it is preferred to use a concentration of about 200 mg eq. of paliperidone per mL or about 312 mg of paliperidone palmitate per mL.

A more preferred procedure involves the addition of a surface modifier to the premix prior to its subjection to mechanical means to reduce the effective average particle size. The concentration of the surface modifier (weight by weight percentage) can vary from about 0.1% to about 90%, preferably from about 0.5% to about 80%, and more preferably is approximately 7% (w/v).

The premix can be used directly by subjecting it to mechanical means to reduce the effective average particle size in the dispersion to the desired particle size. It is preferred that the premix be used directly when a ball mill is used for attrition. Alternatively, the antipsychotic agent and, optionally, the surface modifier, can be dispersed in the liquid medium using suitable agitation such as, for example, a roller mill or a Cowles type mixer, until a homogeneous dispersion is achieved.

The mechanical means applied to reduce the effective average particle size of the antipsychotic conveniently can take the form of a dispersion mill. Suitable dispersion mills include a ball mill, an attritor mill, a vibratory mill, a planetary mill, media mills—such as a sand mill and a bead mill. A media mill is preferred due to the relatively shorter milling time required to provide the desired reduction in particle size. For media milling, in some embodiments, the apparent viscosity of the premix preferably is anywhere between about 0.1 Pa·s and about 1 Pa·s. In some embodiments, for ball milling, the apparent viscosity of the premix preferably is anywhere between about 1 mPa·s and about 100 mPa·s.

The grinding media for the particle size reduction step can be selected from rigid media preferably spherical or particulate in form having an average size less than about 3 mm and, more preferably, less than about 1 mm. Such media desirably can provide the particles of the invention with shorter processing times and impart less wear to the milling equipment. The selection of the material for the grinding media is believed not to be critical. However, about 95% ZrO stabilized with magnesia, zirconium silicate, and glass grinding media provide particles which are acceptable for the preparation of pharmaceutical compositions. Further, other media, such as polymeric beads, stainless steel, titania, alumina and about 95% ZrO stabilized with yttrium, are useful. Preferred grinding media have a density greater than about 2.5 g/cm³ and include about 95% ZrO stabilized with magnesia and polymeric beads.

The attrition time can vary widely and depends primarily upon the particular mechanical means and processing conditions selected. For rolling mills, processing times of up to two days or longer may be required for smaller size particles.

The particles are typically reduced in size at a temperature which does not significantly degrade the antipsychotic agent. Processing temperatures of less than about 30° C. to about 40° C. are ordinarily preferred. If desired, the processing equipment may be cooled with conventional cooling equipment. The method is conveniently carried out under conditions of ambient temperature and at processing pressures which are safe and effective for the milling process.

The surface modifier, if it was not present in the premix, is typically added to the dispersion after attrition in an amount, for example, as described for the premix above. Thereafter, the dispersion can be mixed by, for example, shaking vigorously. Optionally, the dispersion can be subjected to a sonication step using, for example, an ultrasonic power supply.

Aqueous compositions according to the present invention conveniently further comprise a suspending agent and a buffer, and optionally one or more of a preservative and an isotonizing agent. Particular ingredients may function as two or more of these agents simultaneously, e.g. behave like a preservative and a buffer, or behave like a buffer and an isotonizing agent.

Suitable suspending agents (also referred to as physical stabilizers) for use in the aqueous suspensions according to the present invention are cellulose derivatives, e.g. methyl cellulose, sodium carboxymethyl cellulose and hydroxypropyl methyl cellulose, polyvinylpyrrolidone, alginates, chitosan, dextrans, gelatin, polyethylene glycols, polyoxyethylene- and polyoxy-propylene ethers. Preferably sodium carboxymethyl cellulose is used in a concentration of about 0.5 to about 2%, most preferably about 1% (w/v).

Suitable wetting agents preferred from the listed surfactant for use in the aqueous suspensions according to the present invention are polyoxyethylene derivatives of sorbitan esters, e.g. polysorbate 20 and polysorbate 80, lecithin, polyoxyethylene- and polyoxypropylene ethers, sodium deoxycholate. Preferably polysorbate 20 is used in a concentration of about 0.5% to about 3%, more preferably about 0.5% to about 2%, most preferably about 1.1% (w/v).

Suitable buffering agents are salts of weak acids and should be used in amount sufficient to render the dispersion from about pH 6.0 to basic. Preferably, the pH is in a range of from about 6.0 to about 9.0; or in the range of from about 6.0 to about 8.0; or about 6.5 to about 7.5. For example, the pH is in the range of about 6.0 to about 6.5; or from about 6.5 to about 7.0; or from about 7.0 to about 7.5; or from about 7.5 to about 8.0; or from about 8.0 to about 8.5; or from about 8.5 to about 9.0. Particularly preferred is the use of a mixture of disodium hydrogen phosphate (anhydrous) (typically about 0.9% (w/v)) and sodium dihydrogen phosphate monohydrate (typically about 0.6% (w/v)). This buffer also renders the dispersion isotonic and, in addition, less prone to flocculation of the ester suspended therein.

Preservatives are antimicrobials and anti-oxidants which can be selected from the group consisting of benzoic acid, benzyl alcohol, butylated hydroxyanisole, butylated hydroxytoluene, chlorbutol, a gallate, a hydroxybenzoate, EDTA, phenol, chlorocresol, metacresol, benzethonium chloride, myristyl-gamma-piccolinium chloride, phenylmercuric acetate and thimerosal. In particular, it is benzyl alcohol which can be used in a concentration up to about 2% (w/v), preferably up to about 1.5% (w/v).

Isotonizing agents are, for example, sodium chloride, dextrose, mannitol, sorbitol, lactose, sodium sulfate. The suspensions conveniently comprise from about 0% to about 10% (w/v) isotonizing agent. Mannitol may be used in a concentration from about 0% to about 7% more preferably, however, from about 1% to about 3% (w/v), especially from about 1.5% to about 2% (w/v) of one or more electrolytes are used to render the suspension isotonic, apparently because ions help to prevent flocculation of the suspended ester. In particular, electrolytes of the buffer serve as isotonizing agent.

A particularly desirable feature for an injectable formulation relates to the ease with which it can be administered. In particular such an injection should be feasible using a needle as fine as possible in a span of time which is as short as possible. This can be accomplished with the aqueous suspensions of the present invention by maintaining certain viscosities that can be easily taken up in a syringe (e.g. from a vial) and injected through a fine needle. For example, a PP1M viscosity is below about 75 mPa·s, or below about 60 mPa·s at room temperature, and a 23 G, 1 inch needle, or a 22 G, 1½ inch needle is typically used For PP3M, a 22 G, 1½ inch needle, or a 22 G, 1 inch needle is typically used. And for PP6M, a 20 G, 1½ inch needle is typically used.

Ideally, aqueous suspensions according to the present invention will comprise as much paliperidone palmitate as can be tolerated so as to keep the injected volume to a minimum, and as little of the other ingredients as possible.

In particular for PP3M or PP6M, the composition comprises, or consists essentially of, (a) from about 200 to about 500 mg/mL of paliperidone palmitate; (b) from about 2 to about 25 mg/mL of wetting agent; (c) from about 2.5 to about 50 mg/mL of one or more buffering agents; (d) from about 25 to about 150 mg/mL of a suspending agent; (e) optionally up to about 2% (w/v) preservatives; and (f) water q.s. ad 100%. Typically, the PP3M or PP6M composition has a pH of from about 6.0 to about 8.0, preferably about a pH of from 6.5 to about 7.5.

In other embodiments, for PP3M or PP6M, the composition comprises, or consists essentially of, (a) from about 250 to about 400 mg/mL of paliperidone palmitate; (b) from about 5 to about 20 mg/mL of wetting agent; (c) from about 5 to about 25 mg/mL of one or more buffering agents; (d) from about 50 to about 100 mg/mL of a suspending agent; (e) optionally up to about 2% (w/v) preservatives; and (f) water q.s. ad 100%.

In other embodiments, for PP3M or PP6M, the composition comprises, or consists essentially of, (a) from about 280 to about 350 mg/mL of paliperidone palmitate; (b) from about 8 to about 12 mg/mL of wetting agent; (c) from about 5 to about 15 mg/mL of one or more buffering agents; (d) from about 65 to about 85 mg/mL of a suspending agent; (e) optionally up to about 2% (w/v) preservatives; and (f) water q.s. ad 100%.

In certain embodiments, the active ingredient in PP3M or PP6M is paliperidone palmitate (about 312 mg/mL). In certain embodiments, the inactive ingredients in PP3M or PP6M is polysorbate 20 (about 10 mg/mL), polyethylene glycol 4000 (about 75 mg/mL), citric acid monohydrate (about 7.5 mg/mL), sodium dihydrogen phosphate monohydrate (about 6 mg/mL), sodium hydroxide (about 5.4 mg/mL) and water for injection. An exemplified PP3M is disclosed in Example 2. An exemplified PP6M is disclosed in Example 3.

In particular, a composition for PP1M will comprise, or consist essentially of, by weight based on the total volume of the composition: (a) from about 1% to 50% (w/v) of the paliperidone palmitate; (b) from about 0.1% to 5% (w/v) of a wetting agent; (c) one or more buffering agents; (d) from about 0.1% to about 5% (w/v) of a suspending agent; (e) optionally up to about 2% (w/v) preservatives; and (f) water q.s. ad 100%. Typically, the PP1M composition has a pH of from about 6.0 to about 8.0, preferably a pH of from about 6.5 to about 7.5.

A composition PP1M will preferably comprise, or consistent essentially of, by weight based on the total volume of the composition: (a) from about 2% to 40% (w/v) of the paliperidone palmitate; (b) from about 0.25% to 3% (w/v) of a wetting agent; (c) one or more buffering agents; (d) from about 0.25% to about 3% (w/v) of a suspending agent; (e) optionally up to about 2% (w/v) preservatives; and (f) water q.s. ad 100%.

A composition for PP1M will more preferably comprise, or consist essentially of, by weight based on the total volume of the composition: (a) from about 3% to 20% (w/v) of the paliperidone palmitate; (b) from about 0.5% to 2% (w/v) of a wetting agent; (c) one or more buffering agents; (d) from about 0.5% to about 2% (w/v) of a suspending agent; (e) optionally up to about 2% (w/v) preservatives; and (f) water q.s. ad 100%.

In particular for PP1M, the composition comprises, or consists essentially of, (a) from about 50 to about 250 mg/mL of paliperidone palmitate; (b) from about 2 to about 25 mg/mL of wetting agent; (c) from about 2.5 to about 50 mg/mL of one or more buffering agents; (d) from about 5 to about 75 mg/mL of a suspending agent; (e) optionally up to about 2% (w/v) preservatives; and (f) water q.s. ad 100%.

In other embodiments, for PP1M, the composition comprises, or consists essentially of, (a) from about 100 to about 200 mg/mL of paliperidone palmitate; (b) from about 5 to about 20 mg/mL of wetting agent; (c) from about 5 to about 25 mg/mL of one or more buffering agents; (d) from about 10 to about 50 mg/mL of a suspending agent; (e) up to about 2% (w/v) preservatives; and (f) water q.s. ad 100%.

In other embodiments, for PP1M, the composition comprises, or consists essentially of, (a) from about 140 to about 180 mg/mL of paliperidone palmitate; (b) from about 8 to about 16 mg/mL of wetting agent; (c) from about 5 to about 15 mg/mL of one or more buffering agents; (d) from about 20 to about 40 mg/mL of a suspending agent; (e) optionally up to about 2% (w/v) preservatives; and (f) water q.s. ad 100%.

Most preferably, the active ingredient in PP1M is paliperidone palmitate (about 156 mg/mL). Most preferably, the inactive ingredients in PP1M is polysorbate 20 (about 12 mg/mL), polyethylene glycol 4000 (about 30 mg/mL), citric acid monohydrate (about 5 mg/mL), sodium dihydrogen phosphate monohydrate (about 2.5 mg/mL), disodium hydrogen phosphate anhydrous (about 5 mg/mL), sodium hydroxide (about 2.84 mg/mL) and water for injection. An exemplified PP1M is disclosed in Example 1.

Preferably an aqueous suspension will be made under sterile conditions and no preservatives will be used. Appropriate methods to aseptically prepare paliperidone palmitate are described in WO 2006/114384 which is hereby incorporated by reference herein.

The preferred aqueous dosage form contains inactive ingredients that are polysorbate 20, polyethylene glycol 4000, citric acid monohydrate, disodium hydrogen phosphate anhydrous, sodium dihydrogen phosphate monohydrate, sodium hydroxide, and water for injection.

A dose or dosing is typically expressed as milligrams (mg) of paliperidone palmitate.

Regarding six month interval dosing, paliperidone palmitate dosing may also be expressed as mg equivalents (mg eq.) of paliperidone with about 1092 and 1560 mg of paliperidone palmitate being equivalent to about 700 and 1000 mg eq., of paliperidone, respectively. For six-month dosing it is preferred to dose patients with about 700 mg eq. to about 1000 mg eq. paliperidone or about 1092 mg to about 1560 mg paliperidone palmitate.

Regarding three month interval dosing, paliperidone palmitate dosing may also be expressed as mg equivalents (mg eq.) of paliperidone with about 273, 410, 546, and 819 mg of paliperidone palmitate being equivalent to about 175, 263, 350, and 525 mg eq., of paliperidone, respectively. For three-month dosing it is preferred to dose patients with about 175 mg eq. to about 525 mg eq. paliperidone or about 273 mg to about 819 mg paliperidone palmitate.

Regarding one month interval dosing, paliperidone palmitate dosing may also be expressed as mg equivalents (mg eq.) of paliperidone with about 39, 78, 117, 156, and 234 mg of paliperidone palmitate being equivalent to about 25, 50, 75, 100 and 150 mg eq., of paliperidone, respectively. For one month dosing it is preferred to dose patients with about 25 mg eq. to about 150 mg eq. paliperidone or about 39 mg to about 234 mg paliperidone palmitate; or about 100 mg eq. to about 150 mg eq. paliperidone or about 156 mg to about 234 mg paliperidone palmitate, such as about 156 mg paliperidone palmitate or about 234 mg paliperidone palmitate.

The term “antipsychotics” or “antipsychotic drug medication” as used herein means any medication used to decrease or ameliorate the symptoms of psychosis in a person with a psychotic disorder.

The term “psychiatric patient” as used herein, refers to a human, who has been the object of treatment, or experiment for a “mental disorder” and “mental illness” refer to those provided in the Diagnostic and Statistical Manual Fifth Edition (DSM 5), American Psychiatric Association (APA). Those of ordinary skill in the art will appreciate that paliperidone esters (e.g. paliperidone palmitate) can be administered to psychiatric patients for all the known uses of risperidone. These mental disorders include, but are not limited to, schizophrenia; bipolar disorder or other disease states in which psychosis, aggressive behavior, anxiety or depression is evidenced. As set forth in DSM-5, schizophrenia refers to conditions characterized as schizophrenia, schizoaffective disorder and schizophreniform disorders. Bipolar Disorder refers to a condition characterized as a Bipolar Disorder, including Bipolar I and Bipolar Disorder II. The DSM was prepared by the Task Force on Nomenclature and Statistics of the American Psychiatric Association, and provides clear descriptions of diagnostic categories. Pathologic psychological conditions, which are psychoses or may be associated with psychotic features, include, but are not limited to the following disorders that have been characterized in the DSM. Diagnostic and Statistical Manual of Mental Disorders, Revised, 5th Ed. (2013). The skilled artisan will recognize that there are alternative nomenclatures, nosologies, and classification systems for pathologic psychological conditions and that these systems evolve with medical scientific progress. Examples of pathologic psychological conditions which may be treated include, but are not limited to, Mild Intellectual Disability, Moderate Intellectual Disability, Severe Intellectual Disability, Profound Intellectual Disability, Intellectual Disability Severity Unspecified, Autistic Disorders, Rett's Disorder, Childhood Disintegrative Disorders, Asperger's Disorder, Pervasive Developmental Disorder Not Otherwise Specified, Attention-Deficit/Hyperactivity Disorder Combined Type, Attention-Deficit/Hyperactivity Disorder Predominately Inattentive Type, Attention-Deficit/Hyperactivity Disorder Predominately Hyperactive-Impulsive Type, Attention-Deficit/Hyperactivity Disorder NOS, Conduct Disorder (Childhood-Onset and Adolescent Type, Oppositional Defiant Disorder, Disruptive Behavior Disorder Not Otherwise Specified, Solitary Aggressive Type, Conduct Disorder, Undifferentiated Type, Tourette's Disorder, Chronic Motor Or Vocal Tic Disorder, Transient Tic Disorder, Tic Disorder NOS, Alcohol Intoxication Delirium, Alcohol Withdrawal Delirium, Alcohol-Induced Persisting Dementia, Alcohol-Induced Psychotic Disorder with Delusions, Alcohol-Induced Psychotic Disorder with Hallucinations, Amphetamine or Similarly Acting Sympathomimetic Intoxication, Amphetamine or Similarly Acting Sympathomimetic Delirium, Amphetamine or Similarly Acting Sympathomimetic Induced Psychotic with Delusions, Amphetamine or Similarly Acting Sympathomimetic Induced Psychotic with Hallucinations, Cannabis-Induced Psychotic Disorder with Delusions, Cannabis-Induced Psychotic Disorder with Hallucinations, Cocaine Intoxication, Cocaine Intoxication Delirium, Cocaine-Induced Psychotic Disorder with Delusions, Cocaine-Induced Psychotic Disorder with Hallucinations, Hallucinogen Intoxication, Hallucinogen Intoxication Delirium, Hallucinogen-Induced Psychotic disorder with Delusions, Hallucinogen-Induced Psychotic disorder with Delusions, Hallucinogen-Induced Mood Disorder, Hallucinogen-Induced Anxiety Disorder, Hallucinogen-Related Disorder Not Otherwise Specified, Inhalant Intoxication, Inhalant Intoxication Delirium, Inhalant-Induced Persisting Dementia, Inhalant-Induced Psychotic Disorder with Delusions, Inhalant-Induced Psychotic with Hallucinations, Inhalant-Induced Mood Disorder, Inhalant-Induced Anxiety Disorder, Inhalant-Related Disorder Not Otherwise Specified, Opioid Intoxication Delirium, Opioid-Induced Psychotic Disorder with Delusions, Opioid Intoxication Delirium, Opioid-Induced Psychotic Disorder with Hallucinations, Opioid-Induced Mood Disorder, Phencyclidine (PCP) or Similarly Acting Arylcyclohexylamine Intoxication, Phencyclidine (PCP) or Similarly Acting Arylcyclohexylamine Intoxication Delirium, Phencyclidine (PCP) or Similarly Acting Arylcyclohexylamine Induced Psychotic Disorder with Delusions, Phencyclidine (PCP) or Similarly Acting Arylcyclohexylamine Induced Psychotic Disorder with Hallucinations, Phencyclidine (PCP) or Similarly Acting Arylcyclohexylamine Mood Disorder, Phencyclidine (PCP) or Similarly Acting Arylcyclohexylamine Induced Anxiety Disorder, Phencyclidine (PCP) or Similarly Acting Arylcyclohexylamine Related Disorder Not Otherwise Specified, Sedative, Hypnotic or Anxiolytic Intoxication, Sedation, Hypnotic or Anxiolytic Intoxication Delirium, Sedation, Hypnotic or Anxiolytic Withdrawal Delirium, Sedation, Hypnotic or Anxiolytic Induced Persisting Dementia, Sedation, Hypnotic or Anxiolytic-Induced Psychotic Disorder with Delusions, Sedation, Hypnotic or Anxiolytic-Induced Psychotic Disorder with Hallucinations, Sedation, Hypnotic or Anxiolytic-Induced Mood Disorder, Sedation, Hypnotic or Anxiolytic-Induced Anxiety Disorder, Other (or Unknown) Substance Intoxication, Other (or Unknown) Substance-Induced Delirium, Other (or Unknown) Substance-Induced Persisting Dementia, Other (or Unknown) Substance-Induced Psychotic Disorder with Delusions, Other (or Unknown) Substance-Induced Psychotic Disorder with Hallucinations, Other (or Unknown) Substance-Induced Mood Disorder, Other (or Unknown) Substance-Induced Anxiety Disorder, Other (or Unknown) Substance Disorder Not Otherwise Specified, Obsessive Compulsive Disorder, Post-traumatic Stress Disorder, Generalized Anxiety Disorder, Anxiety Disorder Not Otherwise Specified, Body Dysmorphic Disorder, Hypochondriasis (or Hypochondriacal Neurosis), Somatization Disorder, Undifferentiated Somatoform Disorder, Somatoform Disorder Not Otherwise Specified, Intermittent Explosive Disorder, Kleptomania, Pathological Gambling, Pyromania, Trichotillomania, and Impulse Control Disorder NOS, Schizophreniform Disorder, Schizoaffective Disorder, Delusional Disorder, Brief Psychotic Disorder, Shared Psychotic Disorder, Psychotic Disorder Due to a General Medical Condition with Delusions, Psychotic Disorder Due to a General Medical Condition with Hallucinations, Psychotic Disorders Not Otherwise Specified, Major Depression, Single Episode, Severe, without Psychotic Features, Major Depression, Recurrent, Severe, without Psychotic Features, Bipolar Disorder, Mixed, Severe, without Psychotic Features, Bipolar Disorder, Mixed, Severe, with Psychotic Features, Bipolar Disorder, Manic, Severe, without Psychotic Features, Bipolar Disorder, Manic, Severe, with Psychotic Features, Bipolar Disorder, Depressed, Severe, without Psychotic Features, Bipolar Disorder, Depressed, Severe, with Psychotic Features, Bipolar II Disorder, Bipolar Disorder Not Otherwise Specified, Personality Disorders, Paranoid, Personality Disorders, Schizoid, Personality Disorders, Schizotypal, Personality Disorders, Antisocial, and Personality Disorders, Borderline.

The term “therapeutically effective amount” as used herein, means that amount of active compound or pharmaceutical agent that elicits the biological or medicinal response in human that is being sought by a researcher, medical doctor or other clinician, which includes alleviation of the symptoms of the disease or disorder being treated.

Those of skill in the treatment of diseases could determine the effective amount of paliperidone to administer for the treatment of the diseases listed above. By way of example, an effective amount of paliperidone for the treatment of mental disorders would be from about 0.01 mg/kg to about 2 mg/kg body weight per day. For semi-annual dosing it is preferred to dose patients with about 700 mg-eq. to about 1000 mg eq. paliperidone or about 1092 mg to about 1560 mg paliperidone palmitate. The amount of paliperidone palmitate is provided in sufficient amount to provide the equivalent dose of paliperidone after the palmitic acid moiety is removed from the ester (e.g. 1560 mg corresponds to paliperidone 1000 mg). For six-month dosing it is preferred to dose patients with about 700 mg eq. to about 1000 mg eq. paliperidone or about 1092 mg to about 1560 mg paliperidone palmitate.

The disclosure also provides methods for treating schizophrenia in a patient in need thereof, comprising administering an approved drug product comprising PP6M in an amount and manner that is described in a drug product label for the approved drug product and/or in an administration and/or treatment regimen described herein.

The disclosure further relates to a pharmaceutical product comprising a paliperidone palmitate extended-release injectable suspension having a six month dosing interval (PP6M), wherein the pharmaceutical product is packaged, and wherein the package includes a label that identifies the PP6M as a an approved drug product for the treatment of schizophrenia after a patient has been adequately treated with either a one-month paliperidone palmitate extended-release injectable suspension (PP1M) for at least four months or a three-month paliperidone palmitate extended-release injectable suspension following at least one 3-month injection cycle.

The disclosure is also directed to an approved drug product in a prefilled syringe, wherein the approved drug product comprises an extended release injectable suspension of 1092 mg of paliperidone palmitate in a 3.5 ml volume or 1560 mg of paliperidone palmitate in a 5 ml volume.

In certain aspects, methods of selling a drug product comprising PP6M are also provided. The terms “sale” or “selling” as used herein refers to transferring a drug product, e.g., a pharmaceutical composition or a dosage form, from a seller to a buyer. In some embodiments, a drug product label for a reference listed drug for the drug product includes instructions for the treatment of schizophrenia after a patient has been adequately treated with either PP1M for at least four months or PP3M following at least one 3-month injection cycle.

The term “offering for sale,” as used herein, refers to the proposal of a sale by a seller to a buyer for a drug product, e.g., a pharmaceutical composition or a dosage form. These methods comprise offering the drug product for sale.

The term “drug product” is product that contains an active pharmaceutical ingredient that has been approved for marketing by a governmental authority, e.g., the Food and Drug Administration or the similar authority in other countries. In the context of the present disclosure, and as reflected in Example 10, an “approved drug product” as used herein means: a pharmaceutical product, that has been approved by a regulatory or government agency for the treatment of adults having schizophrenia, after they have been adequately treated with either: i) a once-a-month paliperidone palmitate extended-release injectable suspension (e.g., INVEGA SUSTENNA) for at least four months; or ii) an every-three-month paliperidone palmitate extended-release injectable suspension (e.g., INVEGA TRINZA) for at least one three-month cycle, and further comprising paliperidone palmitate administered by gluteal injection once every six months, in either a 1092 milligram or 1560 milligram dose, wherein a relapse event was experienced by 7.5% of a first population of adults having schizophrenia who received said paliperidone palmitate administered by gluteal injection once every six months (PP6M) over a period of twelve months; and 4.9% of a second population of adults having schizophrenia who received an every-three-month paliperidone palmitate extended release injectable suspension (PP3M) over a period of twelve months, respectively, with the Kaplan-Meier estimated difference (PP6M−PP3M) of 2.9% (95% CI: −1.1 to 6.8), the upper bound of the 95% CI (6.8%) being less than 10%, the prespecified non-inferiority margin, thereby demonstrating that said treatment experienced by said first population of adults was non-inferior to said treatment received by said second population of adults. Other elements of the approved product definition are further defined in Example 10, including relapse event that is defined in Section 14 of Example 10.

Therefore, in one aspect, the present disclosure is directed to a method of treating schizophrenia in a patient, wherein the method comprises administering PP6M to the patient. In another aspect, the present disclosure is directed to a method of treating a patient having schizophrenia, wherein the method comprises administering PP6M to the patient.

In one embodiment, the patient has been previously treated with either PP1M for at least four months, or PP3M for at least one 3-month injection cycle. In one embodiment, the patient has been previously treated with PP1M for at least four months. In one embodiment, the patient has been previously treated with PP1M for at least four months at dosages of 156 mg or 234 mg. In one embodiment, the patient has been previously treated with PP1M for at least four months at a dosage of 156 mg. In one embodiment, the patient has been previously treated with PP1M for at least four months at a dosage of 234 mg. In one embodiment, the patient has been previously treated with PP3M for at least one 3-month injection cycle. In one embodiment, the patient has been previously treated with PP3M for at least one 3-month injection cycle at a dosage of 546 mg or 819 mg. In one embodiment, the patient has been previously treated with PP3M for at least one 3-month injection cycle at a dosage of 546 mg. In one embodiment, the patient has been previously treated with PP3M for at least one 3-month injection cycle at a dosage of 819 mg.

In one embodiment, the patient is clinically stable prior to being administered PP6M. In one embodiment, the patient is defined as being clinically stable when the patient has a PANSS total score of less than 70 points prior to being administered PP6M. In one embodiment, the patient is defined as being clinically stable when the patient has a PANSS total scope of less than 70 points for the previous 2 assessments prior to being administered PP6M.

In one embodiment, the patient is an adult patient.

In one embodiment, the patient is administered PP6M once every six months. In one embodiment, PP6M is administered once every six months over a period of 12 months.

In one embodiment, the method of treating schizophrenia is non-inferior to a second method of treating schizophrenia in a second patient, wherein the second method comprises administering PP3M to the second patient over a period of 12 months.

In another aspect, the present disclosure relates to a method of treating schizophrenia in a patient, wherein the method comprises:
(a) administering PP1M or PP3M to the patient; and
(b) subsequently administering PP6M to the patient.

In another aspect, the present disclosure relates to a method of treating a patient with schizophrenia, wherein the method comprises:
(a) administering PP1M or PP3M to the patient; and
(b) subsequently administering PP6M to the patient.

In one embodiment, step (a) comprises administering PP1M to the patient.

In one embodiment, step (a) comprises administering PP3M to the patient.

In one embodiment, the patient is administered PP1M in step (a) and in step (b) is administered PP6M one month (±7 days) after the administration of PP1M.

In one embodiment, the patient is administered PP3M in step (a) and in step (b) is administered PP6M 3 months (±14 days) after the administration of PP3M.

In one embodiment, the patient is not administered any antipsychotic drugs in between steps (a) and (b).

In one embodiment, the method comprises:
(a) administering PP1M for at least 4 months; and
(b) subsequently administering a dose of PP6M to the patient.

In one embodiment, PP1M is administered at dosages of 156 mg or 234 mg. In one embodiment, PP1M is administered at a dosage of 156 mg. In one embodiment, PP1M is administered at a dosage of 234 mg.

In one embodiment, PP3M is administered at dosages of 546 mg or 819 mg. In one embodiment, PP3M is administered at a dosage of 546 mg. In one embodiment, PP3M is administered at a dosage of 819 mg.

In one embodiment, the patient is clinically stable prior to being administered PP6M in step (b). In one embodiment, the patient is defined as being clinically stable when the patient has a PANSS total score of less than 70 points prior to being administered PP6M in step (b). In one embodiment, the patient is defined as being clinically stable when the patient has a PANSS total scope of less than 70 points for the previous 2 assessments prior to being administered PP6M in step (b).

In one embodiment, the patient is an adult patient.

In one embodiment, the patient is administered PP6M in step (b) once every six months. In one embodiment, PP6M is administered in step (b) once every six months over a period of 12 months.

In one embodiment, the method of treating schizophrenia is non-inferior to a second method of treating schizophrenia in a second patient, wherein the second method comprises:
(i) administering PP1M or PP3M to the second patient; and
(ii) administering PP3M to the second patient over a period of 12 months.

Similarly, “label” or “drug product label” refers to information provided to a patient which provides relevant information regarding the drug product. Such information includes, without limitation, one or more of the description of the drug, clinical pharmacology, indications (uses for the drug product), contraindication (who should not take the drug product), warnings, precautions, adverse events (side effects), drug abuse and dependence, dosage and administration, use in pregnancy, use in nursing mothers, use in children and older patients, how the drug is supplied, safety information for the patient, or any combination thereof. In certain embodiments, the label or drug product label provides an instruction for use in a patient requiring antipsychotic medication. In further embodiments, the label or drug product label identifies PP6M and provides instructions for its use in a patient requiring antipsychotic medication.

The term “reference listed drug” or “RLD” as used herein refers to a drug product to which new generic versions are compared to show that they are bioequivalent. It is also a medicinal product that has been granted marketing authorization by a member state of the European Union or by the Commission on the basis of a completed dossier, i.e., with the submission of quality, pre-clinical and clinical data in accordance with Articles 8(3), 10a, 10b or 10c of Directive 2001/83/EC and to which the application for marketing authorization for a generic/hybrid medicinal product refers, by demonstration of bioequivalence, usually through the submission of the appropriate bioavailability studies.

In the United States, a company seeking approval to market a generic equivalent must refer to the RLD in its Abbreviated New Drug Application (ANDA). For example, an ANDA applicant relies on the FDA's finding that a previously approved drug product, i.e., the RLD, is safe and effective, and must demonstrate, among other things, that the generic drug product is the same as the RLD in certain ways. Specifically, with limited exceptions, a drug product for which an ANDA is submitted must have, among other things, the same active ingredient(s), conditions of use, route of administration, dosage form, strength, and (with certain permissible differences) labeling as the RLD. The RLD is the listed drug to which the ANDA applicant must show its ANDA drug product is the same with respect to active ingredient(s), dosage form, route of administration, strength, labeling and conditions of use, among other characteristics. In the electronic Orange Book, there is a column for RLDs and a column for reference standards. In the printed version of the Orange Book, the RLDs and reference standards are identified by specific symbol.

A reference standard is the drug product selected by FDA that an applicant seeking approval of an ANDA must use in conducting an in vivo bioequivalence study required for approval. FDA generally selects a single reference standard that ANDA applicants must use in in vivo bioequivalence testing. Ordinarily, FDA will select the reference listed drug as the reference standard. However, in some instances (e.g., where the reference listed drug has been withdrawn from sale and FDA has determined it was not withdrawn for reasons of safety or effectiveness, and FDA selects an ANDA as the reference standard), the reference listed drug and the reference standard may be different.

FDA identifies reference listed drugs in the Prescription Drug Product, OTC Drug Product, and Discontinued Drug Product Lists. Listed drugs identified as reference listed drugs represent drug products upon which an applicant can rely in seeking approval of an ANDA. FDA intends to update periodically the reference listed drugs identified in the Prescription Drug Product, OTC Drug Product, and Discontinued Drug Product Lists, as appropriate.

FDA also identifies reference standards in the Prescription Drug Product and OTC Drug Product Lists. Listed drugs identified as reference standards represent the FDA's best judgment at this time as to the appropriate comparator for purposes of conducting any in vivo bioequivalence studies required for approval.

In some instances when FDA has not designated a listed drug as a reference listed drug, such listed drug may be shielded from generic competition. If FDA has not designated a reference listed drug for a drug product the applicant intends to duplicate, the potential applicant may ask FDA to designate a reference listed drug for that drug product.

FDA may, on its own initiative, select a new reference standard when doing so will help to ensure that applications for generic drugs may be submitted and evaluated, e.g., in the event that the listed drug currently selected as the reference standard has been withdrawn from sale for other than safety and efficacy reasons.

In Europe, Applicants identify in the application form for its generic/hybrid medicinal product, which is the same as an ANDA or supplemental NDA (SNDA) drug product, the reference medicinal product (product name, strength, pharmaceutical form, marketing authorization holder (MAH, first authorization, Member State/Community), which is synonymous with a RLD, as follows:

1. The medicinal product that is or has been authorized in the European Economic Area (EEA), used as the basis for demonstrating that the data protection period defined in the European pharmaceutical legislation has expired. This reference medicinal product, identified for the purpose of calculating expiry of the period of data protection, may be for a different strength, pharmaceutical form, administration route or presentation than the generic/hybrid medicinal product.

2. The medicinal product, the dossier of which is cross-referred to in the generic/hybrid application (product name, strength, pharmaceutical form, MAH, marketing authorization number). This reference medicinal product may have been authorized through separate procedures and under a different name than the reference medicinal product identified for the purpose of calculating expiry of the period of data protection. The product information of this reference medicinal product will, in principle, serve as the basis for the product information claimed for the generic/hybrid medicinal product.

3. The medicinal product (product name, strength, pharmaceutical form, MAH, Member State of source) used for the bioequivalence study(ies) (where applicable).

The different abbreviated approval pathways for drug products under the Food, Drug, and Cosmetics (FD&C) Act are the abbreviated approval pathways described in sections 505(j) and 505(b)(2) of the FD&C Act (21 U.S.C. 355(j) and 21 U.S.C. 23 355(b)(2), respectively).

According to the FDA (“Determining Whether to Submit an ANDA or a 505(b)(2) Application Guidance for Industry,” U.S. Department of Health and Human Services, October 2017, pp. 1-14, the contents of which is incorporated herein by reference), NDAs and ANDAs can be divided into the following four categories:

(1) A “stand-alone NDA” is an application submitted under section 505(b)(1) and approved under section 505(c) of the FD&C Act that contains full reports of investigations of safety and effectiveness that were conducted by or for the applicant or for which the applicant has a right of reference or use.

(2) A section 505(b)(2) application is an NDA submitted under section 505(b)(1) and approved under section 505(c) of the FD&C Act that contains full reports of investigations of safety and effectiveness, where at least some of the information required for approval comes from studies not conducted by or for the applicant and for which the applicant has not obtained a right of reference or use.

(3) An ANDA is an application for a duplicate of a previously approved drug product that was submitted and approved under section 505(j) of the FD&C Act. An ANDA relies on the FDA's finding that the previously approved drug product, i.e., the reference listed drug (RLD), is safe and effective. An ANDA generally must contain information to show that the generic product (a) is the same as the RLD with respect to the active ingredient(s), conditions of use, route of administration, dosage form, strength, and labeling (with certain permissible differences) and (b) is bioequivalent to the RLD. An ANDA may not be submitted if studies are necessary to establish the safety and effectiveness of the product.

(4) A petitioned ANDA is a type of ANDA for a drug product that differs from the RLD in its dosage form, route of administration, strength, or active ingredient (in a product with more than one active ingredient) and for which FDA has determined, in response to a petition submitted under section 505(j)(2)(C) of the FD&C Act (suitability petition), that studies are not necessary to establish the safety and effectiveness of the drug product.

A scientific premise underlying the Hatch-Waxman Act is that a drug product approved in an ANDA under section 505(j) of the FD&C Act is presumed to be therapeutically equivalent to its RLD. Products classified as therapeutically equivalent can be substituted with the full expectation that the substituted product will produce the same clinical effect and safety profile as the prescribed product when administered to patients under the conditions specified in the labeling. In contrast to an ANDA, a section 505(b)(2) application allows greater flexibility as to the characteristics of the product. A section 505(b)(2) application will not necessarily be rated therapeutically equivalent to the listed drug it references upon approval.

The term “therapeutically equivalent to a reference listed drug” is means that the drug product is a generic equivalent, i.e., pharmaceutical equivalents, of the reference listed drug product and, as such, is rated an AB therapeutic equivalent to the reference listed drug product by the FDA whereby actual or potential bioequivalence problems have been resolved with adequate in vivo and/or in vitro evidence supporting bioequivalence.

“Pharmaceutical equivalents” means drug products in identical dosage forms and route(s) of administration that contain identical amounts of the identical active drug ingredient as the reference listed drug.

FDA classifies as therapeutically equivalent those products that meet the following general criteria: (1) they are approved as safe and effective; (2) they are pharmaceutical equivalents in that they (a) contain identical amounts of the same active drug ingredient in the same dosage form and route of administration, and (b) meet compendial or other applicable standards of strength, quality, purity, and identity; (3) they are bioequivalent in that (a) they do not present a known or potential bioequivalence problem, and they meet an acceptable in vitro standard, or (b) if they do present such a known or potential problem, they are shown to meet an appropriate bioequivalence standard; (4) they are adequately labeled; and (5) they are manufactured in compliance with Current Good Manufacturing Practice regulations

The term “bioequivalent” or “bioequivalence” is the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. Section 505(j)(8)(B) of the FD&C Act describes one set of conditions under which a test and reference listed drug shall be considered bioequivalent:

the rate and extent of absorption of the [test] drug do not show a significant difference from the rate and extent of absorption of the [reference] drug when administered at the same molar dose of the therapeutic ingredient under similar experimental conditions in either a single dose or multiple doses; or

the extent of absorption of the [test] drug does not show a significant difference from the extent of absorption of the [reference] drug when administered at the same molar dose of the therapeutic ingredient under similar experimental conditions in either a single dose or multiple doses and the difference from the [reference] drug in the rate of absorption of the drug is intentional, is reflected in its labeling, is not essential to the attainment of effective body drug concentrations on chronic use, and is considered medically insignificant for the drug.

Where these above methods are not applicable (e.g., for drug products that are not intended to be absorbed into the bloodstream), other scientifically valid in vivo or in vitro test methods to demonstrate bioequivalence may be appropriate.

For example, bioequivalence may sometimes be demonstrated using an in vitro bioequivalence standard, especially when such an in vitro test has been correlated with human in vivo bioavailability data. In other situations, bioequivalence may sometimes be demonstrated through comparative clinical trials or pharmacodynamic studies.

The methods may also comprise, consist of, or consist essentially of selling or offering to sell PP6M into the stream of commerce.

In some embodiments, certain drug interactions are to be avoided with PP6M. For example, concomitant use of centrally acting drugs and alcohol may modulate the CNS effects of PP6M. Accordingly, the methods described herein should avoid concomitant use of alcohol.

In some aspects, a patient may be or become pregnant. For these patients seeking treatment with PP6M, a healthcare provider should be contacted. Moreover, the patient's breastmilk should be monitored for levels of PP6M and infants exposed to PP6M should also be monitored for side effects, such as excess sedation, failure to thrive, jitteriness, and extrapyramidal symptoms (tremors and abnormal muscle movements).

In some aspects, certain activities should be avoided during treatment with PP6M. For example, patients should not drive, or operate heavy machinery. In addition, patients should avoid getting too hot or dehydrated or avoid excessive exercise.

The following Examples are provided to illustrate some of the concepts described within this disclosure. While the Examples are considered to describe certain embodiments, they should not be considered to limit the more general embodiments described herein. The following non-limiting examples are provided to further support the present disclosure. Unless otherwise noted, references to PP1M, PP3M and PP6M in Examples 4-9 refer to the formulations described in Example 1 (PP1M), Example 2 (PP3M) and Example 3 (PP6M).

### EXAMPLE 1

### One-Month Extended Release Formulation (PP1M)

Table 2 below includes an exemplary one-month extended release formulation (PP1M) of 100 mg/mL eq. paliperidone suitable for intramuscular (IM) injection.

TABLE 2
PP1M

| | Concentration |
|---|---|
| Component | (mg/mL) |
| Paliperidone Palmitate | 156 |
| Polysorbate 20 | 12 |
| Polyethylene Glycol 4000¹ | 30 |
| Citric Acid Monohydrate | 5 |
| Di sodium Hydrogen Phosphate, Anhydrous | 5 |
| Sodium Dihydrogen Phosphate Monohydrate | 2.5 |
| Sodium Hydroxide | 2.84 |
| Water for Injection | q.s. ad 1000 pL |

¹ equivalent to PEG 400 or MacroGol 4000

The PP1M can be provided in a prefilled syringe, with dosage strengths ranging from 25 mg eq. to 150 mg eq. obtained by filling the syringes with different volumes of a 100 mg/mL eq. bulk suspension. Table 3 shows the different dosage strengths, including syringe size and nominal fill volume.

TABLE 3
PPIM Dosage Strengths with Syringe Size and Fill Volume

| Strength (mg) | Syringe Size | Nominal Fill Volume (mL) |
|---|---|---|
| eq. 25 | 0.5 mL | 0.250 |
| eq. 50 | 0.5 mL | 0.500 |
| eq. 75 | 1 mL Long | 0.750 |
| eq. 100 | 1 mL Long | 1.000 |
| eq. 125 | 2.25 mL | 1.250 |
| eq.150 | 2.25 mL | 1.500 |

Table 4 describes the syringe components used to package the PP1M.

TABLE 4
Syringe Components for PP1M

| Component | Description |
|---|---|
| Syringe Barrel | Transparent Cyclic Olefin Copolymer (COC) with Integrated |
| | Luer Lock Sizes of 0.5 mL, 1 mL Long or 2.25 mL |
| Tip Cap | Bromobutyl Rubber, Dark Grey |
| Plunger Stopper | FluroTec © Coated Bromobutyl Rubber, Dark Grey (1 mL |
| | Long used for 0.5 mL syringe and 1 mL Long syringe; 1-3 mL |
| | used for 2.25 mL syringe) |

### EXAMPLE 2

### Three Month Extended Release Formulation (PP3M)

Table 5 below includes an exemplary three-month extended release formulation (PP3M) of 200 mg/mL eq. paliperidone suitable for intramuscular (IM) injection.

TABLE 5
PP3M

| | Concentration |
|---|---|
| Component | (mg/mL) |
| Paliperidone Palmitate | 312 |
| Polysorbate 20 | 10 |
| Polyethylene Glycol 4000 | 75 |
| Citric Acid Monohydrate | 7.5 |
| Sodium Dihydrogen | 6 |
| Phosphate Monohydrate | |
| Sodium Hydroxide | 5.4 |
| Water for Injection | q.s. ad 1 mL |

The PP3M can be provided in a prefilled syringe, with dosage strengths ranging from 175 mg eq. to 525 mg eq. obtained by filling the syringes with different volumes of a 200 mg/mL eq. bulk suspension. Table 6 shows the different dosage strengths, including syringe size and nominal fill volume.

TABLE 6
PP3M Dosage Strengths with Syringe Size and Fill Volume

| Dose as | Dose Equivalent | | |
|---|---|---|---|
| Paliperidone | as Paliperidone | | Nominal Fill |
| Palmitate (mg) | (mg) | Syringe Size | Volume (mL) |
| 273 | 175 | 1 mL Long | 0.875 |
| 410 | 263 | 2.25 mL | 1.315 |
| 546 | 350 | 2.25 mL | 1.750 |
| 819 | 525 | 2.8 mL | 2.625 |

Table 7 describes the syringe components used to package the PP3M.

TABLE 7
Syringe Components for PP3M

| Component | Description |
|---|---|
| Syringe Barrel | Transparent Cyclic Olefin Copolymer (COC) with Integrated |
| | Luer Lock Sizes of 1 mL Long, 2.25 mL or 2.8 mL |
| Tip Cap | Bromobutyl Rubber, Dark Grey |
| Plunger Stopper | FluroTec © Coated Bromobutyl Rubber, Dark Grey (1 mL |
| | Long used for 1 mL Long syringe; and 1-3 mL used for 2.25 |
| | mL syringe and 2.8 mL syringe) |

### EXAMPLE 3

### Six Month Extended Release Formulation (PP6M)

Table 8 below includes an exemplary six-month extended release formulation (PP6M) of 200 mg/mL eq. paliperidone palmitate suitable for intramuscular (IM) injection.

TABLE 8
PP6M

| | | Unit Dose | Unit Dose |
|---|---|---|---|
| | Concentration | (mg/syringe in | (mg/syringe in |
| Component | (mg/mL) | 3.5 mL Dose) | 5.0 mL Dose) |
| Paliperidone Palmitate | 312 | 1092 | 1560 |
| Polysorbate 20 | 10 | 35 | 50 |
| Polyethylene Glycol 4000 | 75 | 262.5 | 375 |
| Citric Acid Monohydrate | 7.5 | 26.25 | 37.5 |
| Sodium Dihydrogen | 6 | 21 | 30 |
| Phosphate Monohydrate | | | |
| Sodium Hydroxide | 5.4 | 18.9 | 27 |
| Water for Injection | q.s. ad 1.0 mL | q.s. ad 3.5 mL | q.s. ad 5.0 mL |

The PP6M can be provided in a prefilled syringe, with dosage strengths ranging from 700 mg eq. to 1000 mg eq. obtained by filling the syringes with different volumes of a 200 mg/mL eq. bulk suspension. Table 9 shows the different dosage strengths, including syringe size and nominal fill volume.

TABLE 9
PP6M Dosage Strengths with Syringe Size and Fill Volume

| Dose as | Dose Equivalent | | |
|---|---|---|---|
| Paliperidone | as Paliperidone | Syringe | Nominal Fill |
| Palmitate (mg) | (mg) | Size | Volume (mL) |
| 1092 | 700 | 5 mL | 3.5 |
| 1560 | 1000 | 5 mL | 5.0 |

Table 10 describes the syringe components used to package the six-month extended release formulation.

TABLE 10
Syringe Components for PP6M

| Component | Description |
|---|---|
| Syringe Barrel | Transparent Cyclic Olefin Copolymer |
| | (COC) with Integrated Luer Lock |
| Tip Cap | Bromobutyl Rubber |
| Plunger Stopper | Bromobutyl Rubber |
| Plunger Rod | Polypropylene |
| Backstop (aka Finger Flange) | Homopolypropylene |

### EXAMPLE 4

### A Double-Blind, Randomized, Active-Controlled, Parallel-Group Study of Paliperidone Palmitate 6-Month Formulation

### Study Design

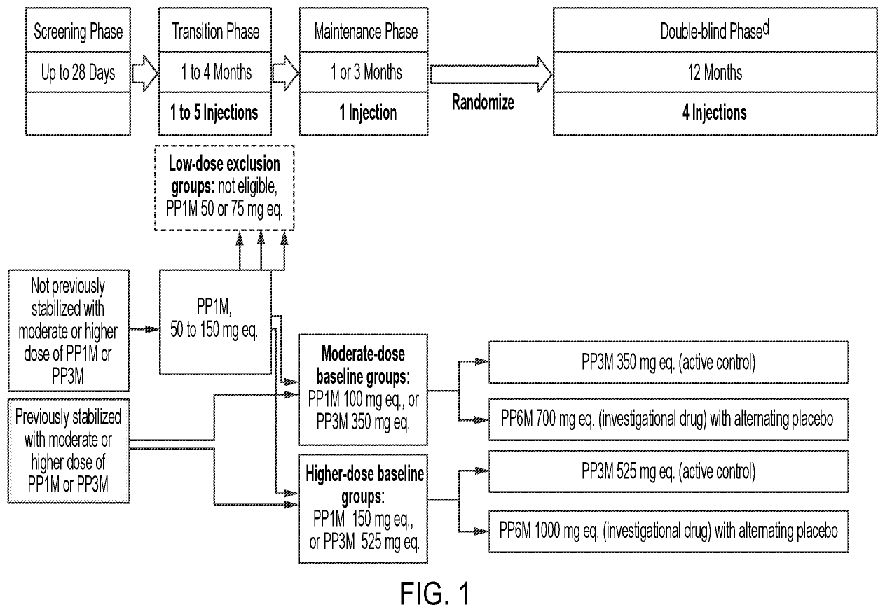

A randomized, double-blind, active-controlled, multicenter, interventional, parallel-group non-inferiority study. A flow chart of the study design is shown in FIG. 1. All eligible subjects who progressed without relapse participated in a Screening Phase (of up to 28 days), a Maintenance Phase that included 1 injection cycle with either PP1M or PP3M (yielding a phase duration of 1 or 3 months, accordingly), and a Double-blind Phase (of 12 months). The Double-blind Phase was designed to include 4 injection cycles of PP3M (active control), or 2 injection cycles of PP6M (investigational drug with alternating placebo).

Before the Maintenance Phase, some subjects participated in a Transition Phase, with 1 to 5 injections of PP1M, if they entered the study on an oral antipsychotic, on injectable risperidone, or on PP1M previously initiated but not yet stabilized. The combined Transition and Maintenance phases is referred to hereafter as the Open-Label Phase.

Randomization: 702 subjects were randomized in a 1:2 ratio to PP3M (n=224) or PP6M (n=478) treatment groups. The randomization was stratified by study center and by the maintenance dose level (moderate or high).

Primary analysis population for efficacy: Double-blind Intent-to-Treat (DB ITT) analysis set, defined as all randomized subjects who received at least 1 dose of double-blind study medication.

Primary efficacy variable: the percentage of subjects who have not relapsed at the end of the 12-month Double-blind Phase based on the Kaplan-Meier cumulative estimate of survival.

Additional Analysis Population for efficacy: Per-protocol analysis set, defined as all randomized subjects who received at least 1 dose of double-blind study medication and did not have major protocol violations, that is, major protocol deviations that may impact efficacy such as violations of intended study population, errors in treatment assignment or use of excluded medication.

Analysis population for safety: same as DB ITT.

Planned sample size: The sample size for the Double-blind Phase of the study was 549 randomized subjects, based on determinations to provide a minimum of 80% power for the primary endpoint. The sample size determination includes the assumptions that the expected survival rate (percentage of subjects remaining relapse-free at 12 months) in the PP3M group is 85%, and that the 1-sided significance level should be 2.5%. Given these assumptions, 549 subjects randomized in a 1:2 ratio (PP3M:PP6M) were required to demonstrate with 80% power that PP6M was no worse than PP3M by a noninferiority margin of 10% for the percentage of subjects remaining relapse-free at 12 months.

### Primary Objective

The primary efficacy objective is to demonstrate that injection cycles consisting of a single administration of PP6M (700 or 1000 mg eq.) are not less effective than 2 sequentially administered injections of PP3M (350 or 525 mg eq.) for the prevention of relapse in subjects with schizophrenia previously stabilized on corresponding doses of PP1M (100 or 150 mg eq.) or PP3M (350 or 525 mg eq.).

### Subject and Treatment Information

The study enrolled 841 subjects across 20 countries and 126 sites. Of those, 702 subjects were randomized to 1 of 2 treatment groups in a 1:2 ratio (224 in PP3M and 478 in PP6M). Among the 702 subjects in the DB ITT population, 23 subjects were excluded from the per-protocol population, the number of subjects included in the per-protocol analysis set is 217 and 462, for the PP3M and PP6M treatment groups, respectively. In the DB ITT analysis set, 521 (74.2%) of the subjects were white and 480 (68.4%) were male. The mean (SD) age was 40.8 (11.53) years, ranging from 18 to 69 years.

Of the 702 randomized subjects, 571 (81.3%) subjects completed the 12-month Double-blind Phase without a relapse event, and 47 (6.7%) subjects completed the Double-blind Phase by having a relapse event. The most frequent reason for withdrawal was ‘Withdrawal by subject’ by 54 (7.7%) subjects.

### Efficacy

The primary efficacy endpoint was the percentage of subjects who have not relapsed by the end of the 12-month Double-blind Phase based on the Kaplan-Meier 12-Month cumulative estimate of survival. Statistical analysis tests were conducted at the two-sided 0.05 significance level.

### Primary Efficacy Endpoint

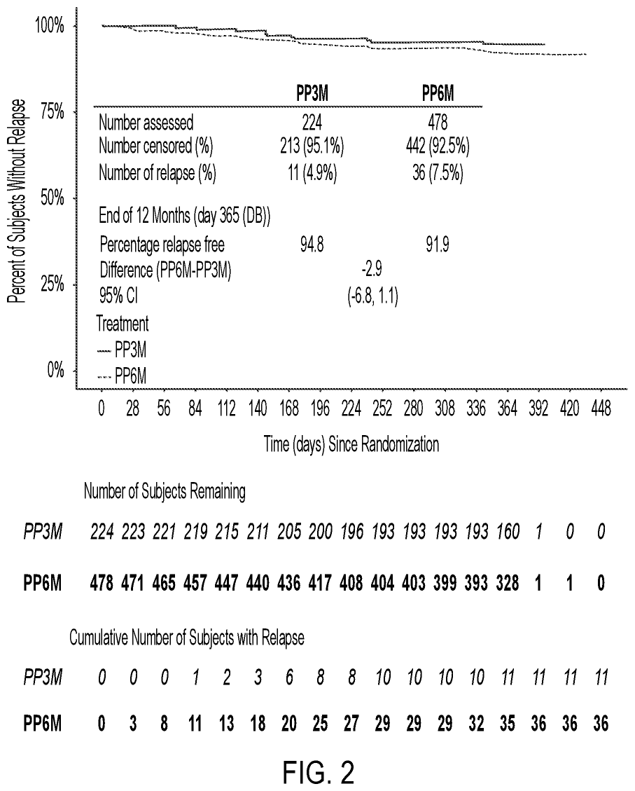

In the DB ITT population, 11 (4.9%) subjects in the PP3M group and 36 (7.5%) subjects in the PP6M group experienced a relapse event during the 12 Month Double-blind Phase. The estimated difference (95% CI) between the treatment groups (PP6M−PP3M) in percentages of subjects who remained relapse free is −2.9% (−6.8%, 1.1%). The lower bound of the 95% confidence interval is larger than the pre-specified non-inferiority margin of −10%, therefore, PP6M can be declared to be non-inferior to PP3M (FIG. 2).

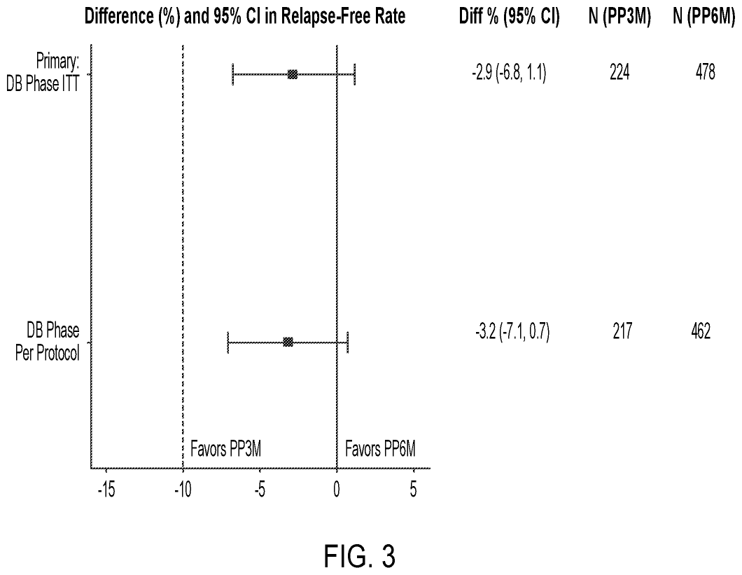

In the per-protocol analysis population, 10 (4.6%) subjects in the PP3M group and 35 (7.6%) subjects in the PP6M group experienced a relapse event during the Double-blind Phase. The results are similar to that obtained for the DB ITT analysis population, further confirming the non-inferiority of PP6M to PP3M (FIG. 3).

Supplementary analyses were conducted for the primary efficacy analysis by including data collected during the follow-up phase for subjects who withdrew from the Double-blind phase. Results are consistent with primary efficacy analysis.

For the DB ITT analysis population, the ratio (95% CI) of the instantaneous risk (hazard) of relapse for a subject in the PP6M treatment group during the Double-blind Phase versus the risk for a subject in the PP3M in the Double-blind Phase was 1.57 (95% CI: 0.8, 3.08), based upon a Cox Proportional Hazards Model with treatment as the only factor. Accordingly, the hazard rate in the PP6M subjects is 1.57 times the hazard rate of PP3M treated subjects.

### Safety

Overall, 297/478 (62.1%) subjects in the PP6M group and 131/224 (58.5%) subjects in the PP3M group experienced at least one TEAE during the Double-blind Phase. The most common (≥5%) TEAEs during the Double-blind Phase were weight increased (8.4%), injection site pain (7.7%), headache (6.7%), upper respiratory tract infection (5.0%) for the PP6M group, and weight increased (7.6%), nasopharyngitis (5.8%), headache (5.4%) for the PP3M group.

There were 1 and 3 deaths in the Open-Label (combined Transition and Maintenance phases) and Double-blind Phases, respectively. Among the 3 deaths in the Double-blind Phase, 1 (0.2%) was for the PP6M group, and 2 (0.9%) were for the PP3M group.

Thirty-nine subjects (24 [5.0%] in PP6M, 15 [6.7%] in PP3M) experienced serious TEAEs during the Double-blind Phase.

During the Double-blind Phase, study medication was permanently stopped due to an adverse event with the following incidence across treatment groups: 16 (3.3%) subjects in the PP6M group, and 6 (2.7%) subjects in the PP3M group.

### Abbreviations

DB: double-blind.

OL: open-label.

MA: maintenance.

PANSS: positive and negative syndrome scale for schizophrenia.

PP: per-protocol.

KM: Kaplan-Meier.

ITT: intent to treat.

SD: standard deviation.

CI: confidence interval.

TEAE: treatment-emergent adverse event.

### EXAMPLE 5

### Dosing Conversion

Conversion from PP1M or PP3M to PP6M doses are described in Table 11 below.

TABLE 11
Paliperidone palmitate Dosing Conversion Tables

PP6M Doses for Adult Patients Adequately Treated with PP1M

| | |
|---|---|
| If the Last Dose of PP1M is: | Transition to PP6M dose of: |
| 156 mg (100 mg eq.) | 1092 mg (700 mg eq.) |
| 234 mg (150 mg eq.) | 1560 mg (1000 mg eq.) |

PP6M Doses for Adult Patients Adequately Treated with PP3M

| | |
|---|---|
| If the Last Dose of PP3M is: | Transition to PP6Mdose of: |
| 546 mg (350 mg eq.) | 1092 mg (700 mg eq.) |
| 819 mg (525 mg eq.) | 1560 mg (1000 mg eq.) |

Patients who are adequately treated with either PP1M (after at least 4 months of treatment) or PP3M (at least one 3-month injection cycle) and do not require dose adjustment may be switched to PP6M. PP6M should be initiated in place of the next scheduled dose of PP1M (±7 days) or PP3M (±14 days). The dose of PP6M should be based on the previous corresponding dose of PP3M or PP1M, as shown in Table 11, supra. When transitioning to PP6M from PP1M, to establish a consistent maintenance dose, it is recommended that the last two doses of PP1M be the same dosage strength before starting PP6M.

A prior treatment period with PP1M or PP3M ensures that paliperidone plasma concentrations are at or approach steady state prior to the transition to PP6M.

Model-based simulations suggest that subjects transitioning directly from PP1M (after at least 4-months of treatment) to PP6M have similar paliperidone exposure levels when compared to those subjects who transition from PP3M (after at least one 3-month injection cycle) to PP6M. Consequently, subjects may be transitioned directly from PP1M to PP6M, without transitioning to PP3M first prior to starting PP6M dosing.

### EXAMPLE 6

### Pharmacokinetic Profile of PP6M in Subjects Transitioning from PP1M or PP3M

### Objective

The objective of this trial was to assess the pharmacokinetic (PK) profile of PP6M (700 or 1000 mg eq.) administered in a gluteal muscle in subjects with schizophrenia who have transitioned from corresponding doses of PP1M (100 or 150 mg eq.) or PP3M (350 or 525 mg eq.).

### Subjects and Methods

This clinical trial was a randomized, double-blind, active-controlled, multicenter, interventional, parallel-group study. All eligible subjects who progressed without relapse participated in a Screening Phase (of up to 28 days), a Maintenance Phase that included 1 injection cycle with either paliperidone palmitate 1-month (PP1M) or paliperidone palmitate 3-month (PP3M) (yielding a phase duration of 1 or 3 months, accordingly), and a double-blind Phase (of 12 months). The double-blind Phase was designed to include 2 injection cycles of paliperidone palmitate 6 month (PP6M) (investigational drug with alternating placebo) or 4 injection cycles of PP3M (active control). Multiple pharmacokinetic blood samples were collected during the open label phase (PP1M and PP3M) as well as double-blind phase (PP3M and PP6M) of the trial to determine the time course of paliperidone plasma concentrations. The aim of the PK evaluations was to characterize the time course of plasma paliperidone concentrations and PK parameters such as maximum and minimum plasma concentrations and their associated timing. Therefore, 3 PK samples were scheduled weekly around the expected paliperidone peak at approximately 1 month after the PP6M dose, and 6 PK samples were scheduled weekly when approaching the end of the 6-month dosing interval.

### Results

Pharmacokinetics of Paliperidone in the Maintenance Phase after PP1M and PP3M Dosing

After administration of PP1M in the Maintenance Phase, median tₘₐₓ after a 100 mg eq. dose was 8 days and was comparable to median tₘₐₓ of 7 days after a 150 mg eq. dose. After administration of 350 or 525 mg eq. (PP3M) median tₘₐₓ was comparable and was 28 days. Based on visual inspection, C_{trough}, Cₘₐₓ, and AUC_{3M} seemed to increase dose proportionally for both PP1M and PP3M. Dose-normalized mean C_{trough}, Cₘₐₓ, and AUC_{3M} were comparable for PP1M and PP3M. The Peak/Trough Ratio was also comparable for PP1M and PP3M.

### Pharmacokinetics of Paliperidone in the Double-Blind Phase After PP6M and PP3M Dosing.

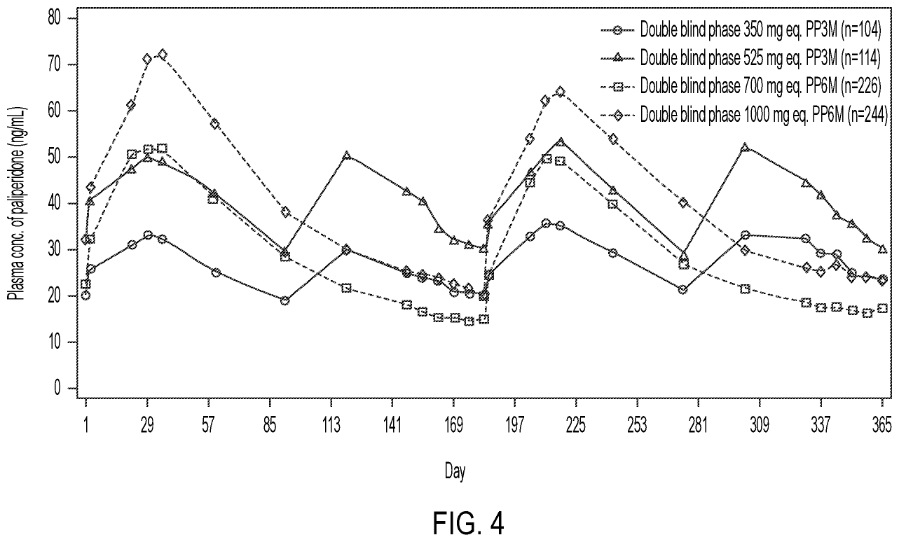

Mean dose-normalized trough concentrations were comparable for PP3M and PP6M at Day 1 (24.6 ng/mL and 25.0 ng/mL, respectively); at later timepoints the subjects who received PP6M had approximately 25-28% lower trough concentrations (16.7 ng/mL at Day 183 and 17.3 ng/mL at Day 365) compared to subjects who received PP3M (22.2 ng/mL at Day 183 and 24.1 ng/mL at Day 365). After the first administration of 350 or 525 mg eq. PP3M or 700 or 1000 mg eq. PP6M in the Double-blind Phase, median tₘₐₓ was comparable for all treatments, i.e., approximately 28 days. Similarly, after administration of 350 or 525 mg eq. PP3M or 700 or 1000 mg eq. PP6M in the second 6 months of the Double-blind Phase, median tₘₐₓ was comparable and ranged between 29 and 32 days. Based on visual inspection, C_{trough}, Cₘₐₓ, and AUC_{6M} seemed to increase dose proportionally for PP6M (700 or 1000 mg eq.) after administration of each, the first and second dose, in the double-blind phase. Similarly, PK exposure parameters for paliperidone (C_{trough}, Cₘₐₓ, and AUC_{6M}) seem to be dose proportional, after the first and third doses of PP3M doses (350 or 525 mg eq.) in the Double-blind Phase. Dose-normalized mean Cₘₐₓ was slightly higher (1.4 to 1.5-fold) for PP6M, when compared to PP3M. Mean dose normalized total paliperidone exposure (AUC_{6M}) was comparable in the Double-blind Phase after PP3M and PP6M dosing. The results are summarized below in Table 12, as well as in FIG. 4.

Median peak-to-trough ratios after PP3M administration in the Maintenance and Double-blind Phase were comparable across doses, ranging from 1.85 to 1.92 and 1.66 to 2.11 in the Maintenance- and Double-blind Phases, respectively. Median peak-to-trough ratios in the double blind phase after PP6M administrated once every six months ranged from 2.71-3.41. Median peak to trough ratios after PP6M administration in the Double-blind Phase were comparable across doses and were slightly higher after the first administration (ranging from 3.32 to 3.41) compared to the second administration (ranging from 2.71 to 3.20).

After stratification per administered dosage, maintenance phase product, injection site in maintenance phase, gender, age, and creatinine clearance category for several groups, no clinically meaningful difference was observed as the ranges were overlapping due to the high inter-subject variation for the PP3M and PP6M subgroups for Cₘₐₓ, AUC_{6M}.

Dose normalized mean paliperidone exposure (Cₘₐₓ, AUC_{6M}) after PP6M administration in the Double-blind Phase was comparable between sub-groups of subjects who receive PP1M or PP3M in the maintenance phase.

TABLE 12
PK Data for Patients Given PP3M and PP6M

| | | | | |
|---|---|---|---|---|
| Paliperidone PK | | | | |
| (mean [SD], | | | | |
| tₘₐₓ:median | PP3M | PP3M | PP6M | PP6M |
| [range]) | (350 mg eq.) | (525 mg eq.) | (700 mg eq.) | (1000 mg eq.) |

DB 0-6 Months

| | | | | |
|---|---|---|---|---|
| n | 98^{a} | 112^{b} | 222^{c} | 229^{d} |
| tₘₐₓ (h) | 670.80 | 679.92 | 671.09 | 674.00 |
| | (0.00-2256.57) | (0.00-2325.15) | (0.00-4367.42) | (0.00-4366.57) |
| tₘₐₓ (days) | 27.95 | 28.33 | 27.96 | 28.08 |
| | (0.00-94.02) | (0.00-96.88) | (0.00- 181.98) | (0.00- 181.94) |
| C_{trough} (ng/mL) | 19.8 (9.82) | 34.1 (19.7) | 17.2 (11.5) | 23.2 (16.2) |
| Cₘₐₓ (ng/mL) | 42.5 (23.7) | 67.0 (39.1) | 68.8 (40.4) | 93.6 (61.2) |
| AUC_{3M} (ng.h/mL) | 64357 (31797) | 103499 (51173) | | |
| AUC_{6M} (ng.h/mL) | 128713 (63593) | 206998 (102347) | 152555 (73249) | 204527 (97213) |

DB 6-12 Months

| | | | | |
|---|---|---|---|---|
| n | 87^{e} | 101^{f} | 193^{g} | 197^{h} |
| tₘₐₓ (h) | 766.17 | 692.33 | 717.87 | 720.45 |
| | (23.67-2301.80) | (44.62-2233.83) | (43.33 -4367.33) | (0.00-3623.42) |
| tₘₐₓ (days) | 31.92 | 28.85 | 29.91 | 30.02 |
| | (0.99-95.91) | (1.86-93.08) | (1.81-181.97) | (0.00-150.98) |
| C_{trough} (ng/mL) | 22.7 (10.8) | 34.8 (20.6) | 17.6 (11.7) | 24.3 (12.8) |
| Cₘₐₓ (ng/mL) | 44.1 (21.1) | 67.2 (55.1) | 67.9 (69.8) | 84.2 (47.0) |
| AUC_{3M} (ng.h/mL) | 68410 (27774) | 103004 (57770) | — | — |
| AUC_{6M} (ng.h/mL) | 136819 (55549) | 206009 (115541) | 143258 (66364) | 191933 (81831) |

^{a}n = 92 for C_{trough} and n = 97 for AUC_{3M} and AUC_{6M}
^{b}n = 108 for C_{trough}
^{c}n = 182 for C_{trough} and n = 215 for AUC_{6M}
^{d}n = 181 for C_{trough} and n = 222 for AUC_{6M}
^{e}n = 82 for C_{trough} and n = 84 for AUC3M and AUC_{6M}
^{f}n = 95 for C_{trough}
^{g}n = 160 for C_{trough} and n = 185 for AUC_{6M}
^{h}n = 177 for C_{trough} and n = 194 for AUC_{6M}

### EXAMPLE 7

### Dosing Window for PP6M Maintenance Treatment

Population PK Simulations: Effects of Extending or Shortening the Dosing Interval on the Cₘₐₓ and C_{trough}

Acceptability of a dosing window 2 weeks before and 3 weeks after the regularly scheduled 6-month maintenance injection was evaluated as follows:

The moderate PP6M dose strength (700 mg eq.) was used to simulate the worst-case scenario where extending the dosing interval results in the lower C_{trough}. As shown in Table 13, for injections delayed by 1, 2 and 3 weeks relative to the scheduled 6-month injection after reaching PP6M steady state on 700 mg eq., the median C_{trough} decreased from 15.8 ng/mL to 15.3 (−3.2%), 14.9 (−5.6%), and 14.4 (−8.9%) ng/mL, respectively; and

The highest PP6M dose strength (1000 mg eq.) was used to simulate the worst-case scenario where shortening the dosing interval results in the highest Cₘₐₓ. As shown in Table 13, reproduced below, for injections administered 1 week earlier and 2 weeks earlier relative to the scheduled 6-month injection after reaching PP6M steady state on 1000 mg eq., the median Cₘₐₓ increased from 76.1 ng/mL to 76.3 (+0.3%) and to 76.6 (+0.7%), respectively.

TABLE 13
Pharmacokinetic Data at PP6M Steady State (dosing 1-2 weeks early
and 1-3 weeks late relative to the scheduled 6-month injection)

| | | Cₘₐₓ | % change | C_{trough} | % change |
|---|---|---|---|---|---|
| Dose | Regime | (ng/mL)^{a} | vs base | (ng/mL)^{b} | vs base |
| High Dose | base PP6M | 76.1 | — | | |
| | 1 week earlier | 76.3 | +0.3% | | |
| | 2 weeks earlier | 76.6 | +0.7% | | |
| Moderate Dose | base PP6M | | | 15.8 | — |
| | 1 week later | | | 15.3 | −3.2% |
| | 2 weeks later | | | 14.9 | −5.7% |
| | 3 weeks later | | | 14.4 | −8.9% |

### Duration of Clinical Effect Based on Median Time to Relapse in Relapse Prevention Studies

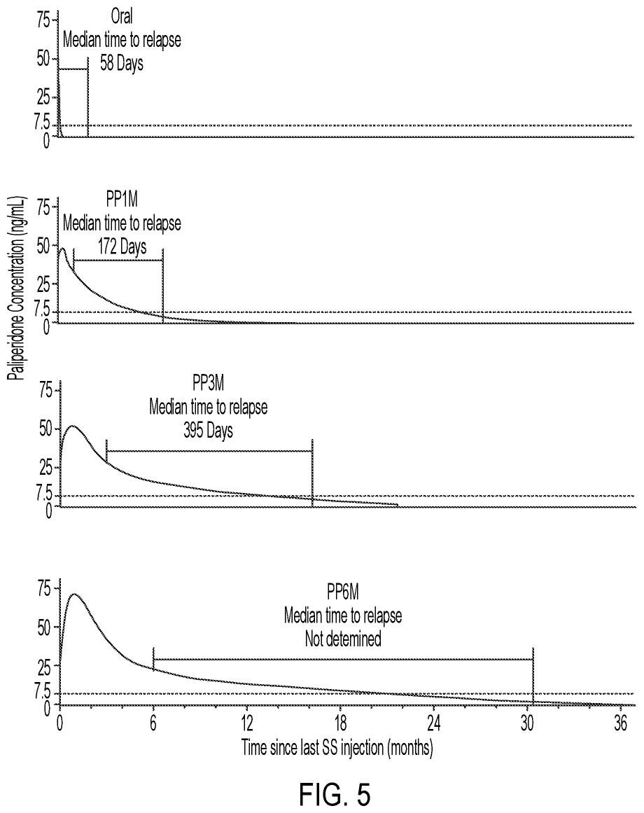

PK simulations were conducted to evaluate the relationship between median time to relapse and the time point at which the median paliperidone concentration decreased to 7.5 ng/mL, following administration of the last steady-state dose in each study prior to the Double-blind phase (oral paliperidone ER 12 mg, PP1M 150 mg eq, PP3M 525 mg eq, and PP6M 1000 mg eq.), as shown in FIG. 5. An apparent delay lasting from several weeks to several months was observed between the time point when the median plasma paliperidone concentration decreased to 7.5 ng/mL and the median time to relapse, i.e., the time point when half of the subjects had experienced relapse, while the other half of the subjects either relapsed later or did not relapse during the study. Thus, it appears that the therapeutic effect is more prolonged than the expected effect based on the 7.5 ng/mL threshold, and the relapse protection window extends farther in the positive direction.

Regarding FIG. 5, the simulations depict the decay in paliperidone plasma concentrations after stopping steady-state dose administrations of: 1) Oral paliperidone ER, 12 mg; 2) PP1M 150 mg eq.; 3) PP3M 525 mg eq.; and 4) PP6M 1000 mg eq.; using the high dose level for each formulation as a representative scenario. Median time to relapse was calculated from the placebo group from the following studies: oral paliperidone ER (R076477SCH301), PP1M (R092670PSY3001), and PP3M (R092670PSY3012) based on the final Kaplan-Meier estimates.

Therefore, a dosing window of up to 2 weeks earlier and up to 3 weeks later than the target 6-month date for maintenance treatment with PP6M is possible and provides scheduling flexibility and enhances treatment adherence, without loss of efficacy or worsening of side effects.

### EXAMPLE 8

### Missed Dosing

Based on population pharmacokinetic simulations, the following guidelines are provided in the event of missed doses of PP6M beyond the dosing-window: If more than 6 months and 3 weeks up to but less than 8 months have elapsed since the last injection of PP6M, the following re-initiation regimen may be used.

TABLE 14
Re-initiation Regimen After Missing over 6 Months
and 3 Weeks up to but less than 8 Months of PP6M

| | | Then administer PP6M |
|---|---|---|
| | Administer PP1M | into gluteal muscle |
| If the Last Dose | into deltoid muscle | 1 month later |
| of PP6M was: | Day 1 | 1 month after Day 1 |
| 1092 mg (700 mg eq.) | 156 mg (100 mg eq.) | 1092 mg (700 mg eq.) |
| 1560 mg (1000 mg eq.) | 234 mg (150 mg eq.) | 1560 mg (1000 mg eq.) |

If 8 months up to and including 11 months have elapsed since the last injection of PP6M, the following re-initiation regimen may be used.

TABLE 15
Re-initiation Regimen After Missing over 8 Months up to 11 Months of PP6M

| | Administer PP1M | | Then administer PP6M |
|---|---|---|---|
| If the last dose | into deltoid muscle | | into gluteal muscle |
| of PP6M was: | Day 1 | Day 8 | 1 month after Day 8 |
| 1092 mg (700 mg eq.) | 156 mg (100 mg eq.) | 156 mg (100 mg eq.) | 1092 mg (700 mg eq.) |
| 1560 mg (1000 mg eq.) | 156 mg (100 mg eq.) | 156 mg (100 mg eq.) | 1560 mg (1000 mg eq.) |

If more than 11 months have elapsed since the last injection of PP6M, re-initiate treatment with PP1M as described in prescribing information for the PP1M product. PP6M can then be resumed after the patient has been adequately treated with PP1M for at least 4 months. To establish a consistent maintenance dose, it is recommended that the last two doses of PP1M should be the same dose strength before re-starting PP6M.

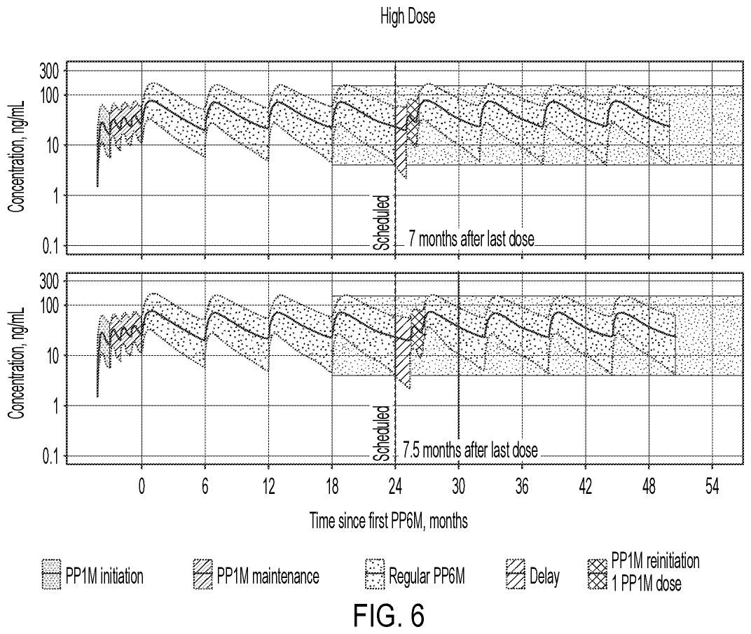

The re-initiation regimen after missed dosing and the timing of continuation of the PP6M maintenance regime depends on the time interval since the last PP6M dose. These recommendations are based on simulations performed to address the scenario of a missed dose in patients that have been stabilized on treatment with PP6M, as shown in FIGS. 6-8. Criteria were to achieve a quick return to paliperidone plasma concentrations as before the missed dose, without creating an overshoot due the applied re-initiation regimen.

Regarding FIG. 6, the middle solid line represents the median paliperidone concentration and the shaded area between the bottom and top dotted lines represents the 90% prediction band. Standard PP1M 4-month treatment in deltoid (initiation doses followed by maintenance doses) followed by PP6M dosing. The delay in the last PP6M dose is indicated, and re-initiation, performed with one dose of 150 mg eq. PP1M in deltoid for the high dose level, is indicated. The light stipple area represents the range from trough to peak concentration (defined by the 90% prediction band) before the PP6M dosing interval changed.

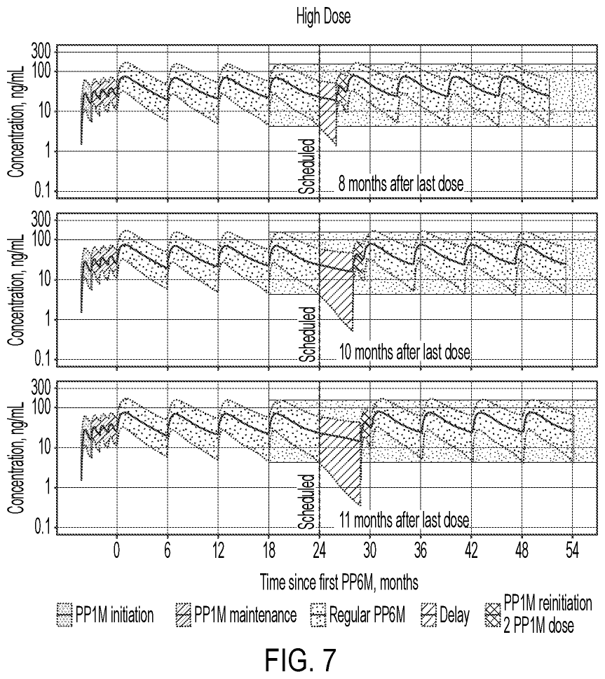

Regarding FIG. 7, the middle solid line represents the median paliperidone concentration and the shaded area between the bottom and top dotted lines represents the 90% prediction band. Standard PP1M 4-month treatment in deltoid (initiation doses followed by maintenance doses) followed by PP6M dosing. The delay in the last PP6M dose is indicated, and re-initiation, performed with two doses of 100 mg eq. PP1M in deltoid, is indicated. The light stipple area represents the range from trough to peak concentration (defined by the 90% prediction band) before the PP6M dosing interval changed.

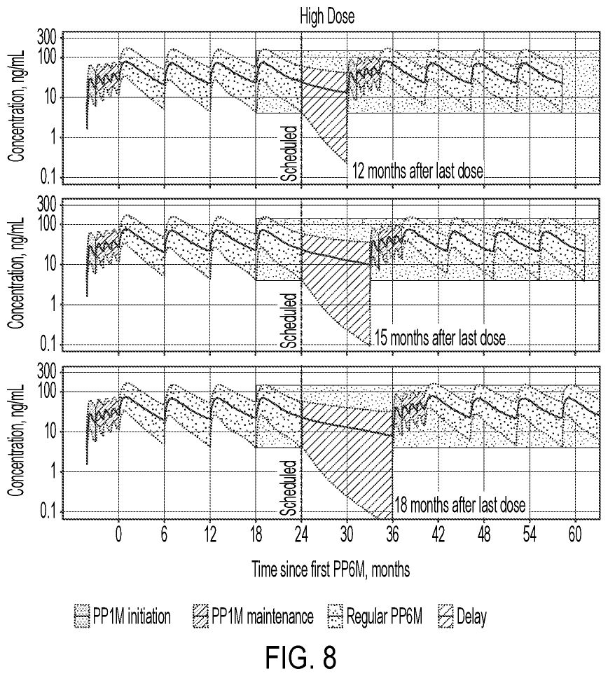

Regarding FIG. 8, the middle solid line represents the median paliperidone concentration and the shaded area between the bottom and top dotted lines represents the 90% prediction band. Standard PP1M 4-month treatment in deltoid (initiation doses followed by maintenance doses) followed by PP6M dosing. The delay in the last PP6M dose is indicated, and re-initiation is performed as a 4-month PP1M treatment in deltoid. The light stipple area represents the range from trough to peak concentration (defined by the 90% prediction band) before the PP6M dosing interval changed.

These guidelines provide a mechanism by which patients can resume treatment with PP6M in case they become fully or partially non-adherent, thereby reducing the need to start treatment de novo.

### EXAMPLE 9

### Weight Change Associated with PP6M Treatment

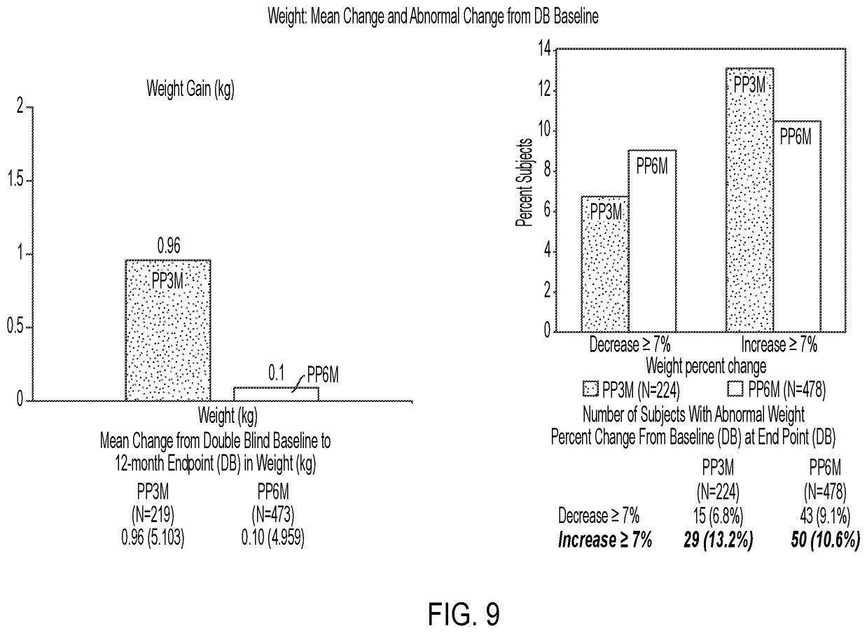

Based on the findings of the studies set forth in Example 4, patients with schizophrenia stabilized on shorter acting paliperidone formulations (PP1M, PP3M) who were switched to the longer acting formulation (PP6M) showed substantially less overall weight gain during double-blind phase (12 months) and more weight loss compared to patients treated with PP3M (active comparator) during the 12 month double-blind phase, as shown in FIG. 9. For example, the weight gain in the patient population of PP6M was negligible (0.1 kg in 12 months, left graph of FIG. 9) and a higher percentage of patients showed a major weight loss of over 7% of their body weight (right graph of FIG. 9).

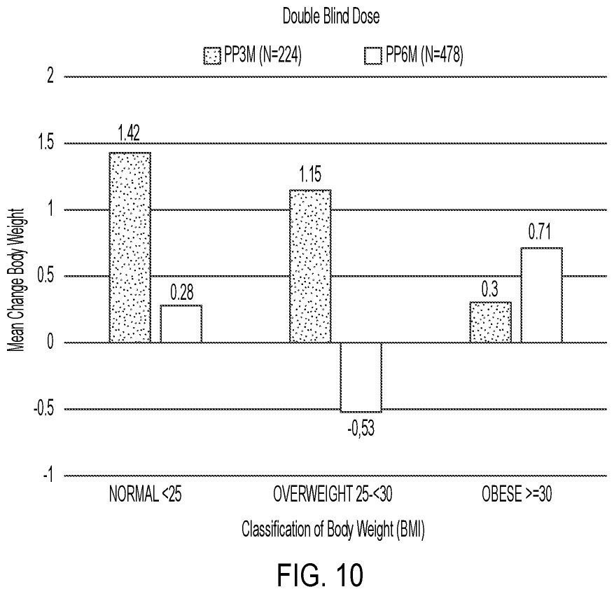

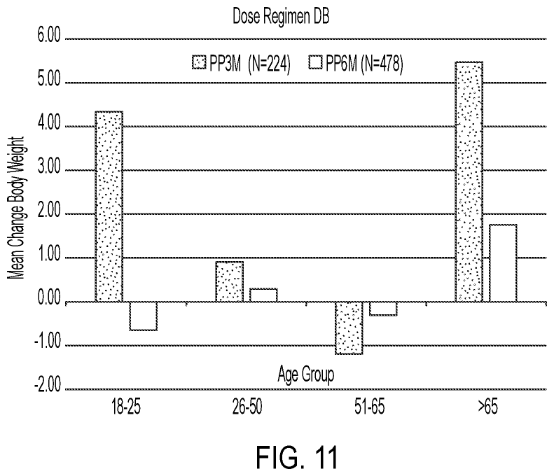

The data was further analyzed and found that overweight patients (BMI between 25 and <30) received a benefit from being switched to PP6M (FIG. 10) as did patients in the age group 18-25 (FIG. 11). While showing a beneficial effect on weight, the studies also showed non-inferior efficacy of PP6M compared to PP3M on the primary endpoint of time to relapse at the end of the 12-month period in both intent-to-treat and per-protocol analysis sets. The safety profile observed for PP6M was consistent with previous studies of PP1M and PP3M formulations with no new safety signals emerging.

As most of the weight gain was reported at the double-blind baseline (day 1) of the study indicating that weight increase occurred during the open label stabilizing phase. There was no incremental weight gain noted during the double-blind phase (12 month) suggesting a stabilizing effect on mean weight gain with more infrequent injections. Therefore, patients with increasing weight could be switched to PP6M to help stabilize their weight or to support a body weight decrease.

From baseline (DB) to double blind end point, the changes in body weight, waist circumference, and BMI were numerically higher in the PP3M group versus the PP6M group. The mean (SD) increases from baseline (MA) to Double-blind end point in body weight were 0.10 (4.959) kg and 0.96 (5.103) kg for the PP6M and PP3M groups, respectively.

From DB baseline to double-blind end point, 10.6% of subjects in the PP6M group and 13.2% of subjects in the PP3M group experienced an abnormal increase in body weight (≥7%). 9.1% of subjects in the PP6M group and 6.8% of subjects in the PP3M group experienced an abnormal decrease in body weight (≥7%) from DB baseline to Double-blind end point.

Regarding FIG. 10, the mean (SD) change by baseline (DB) BMI was 0.28 (3.404) kg in the PP6M group and 1.42 (4.456) kg in the PP3M group for subjects with normal (<25) baseline BMI; −0.53 (4.386) kg in the PP6M group and 1.15 kg (4.814) kg in the PP3M group for over-weight (BMI 25 to <30) subjects; and 0.71 (6.448) kg in the PP6M group and 0.30 (5.955) kg in the PP3M group for obese (BMI ≥30) subjects.

Regarding FIG. 11, the mean (SD) change by age was −0.65 (4.955) kg in the PP6M group and 4.33 (7.112) kg in the PP3M group for subjects in the 18 to 25 years age group; 0.29 (4.878) kg in the PP6M group and 0.91 (4.600) kg in the PP3M group for subjects in the 25 to 50 years age group; −0.31 (5.247) kg in the PP6M group and −1.20 (4.763) kg in the PP3M group for subjects in the 51 to 65 years age group; and 1.76 (4.738) kg in the PP6M group and 5.47 (5.707) kg for subjects >65 years.

### EXAMPLE 10

### Approved Drug Product Label

### Highlights of Prescribing Information

These highlights do not include all the information needed to use INVEGA HAFYERA safely and effectively. See full prescribing information for INVEGA HAFYERA.

INVEGA HAFYERA™ (paliperidone palmitate) extended-release injectable suspension, for gluteal intramuscular use.

Initial U.S. Approval: 2006.

WARNING: INCREASED MORTALITY IN ELDERLY PATIENTS WITH DEMENTIA-RELATED PSYCHOSIS

See full prescribing information for complete boxed warning.

Elderly patients with dementia-related psychosis treated with antipsychotic drugs are at an increased risk of death. INVEGA HAFYERA is not approved for use in patients with dementia-related psychosis. (5.1).

### Indications and Usage

INVEGA HAFYERA, an every-six-month injection, is an atypical antipsychotic indicated for the treatment of schizophrenia in adults after they have been adequately treated with:
A once-a-month paliperidone palmitate extended-release injectable suspension (e.g., INVEGA SUSTENNA) for at least four months or
An every-three-month paliperidone palmitate extended-release injectable suspension (e.g., INVEGA TRINZA) for at least one three-month cycle. (1)

### Dosage and Administration

Administer INVEGA HAFYERA by gluteal injection once every 6 months by a healthcare professional. Do not administer by any other route. (2.1)
See Full Prescribing Information for complete dosing information. (2.2)
Initiate INVEGA HAFYERA when the next once-a-month or every three-month paliperidone palmitate extended-release injectable suspension dose is scheduled. Dose is based on the previous once-a-month or every-three-month product. (2.2):

INVEGA HAFYERA Doses for Adults Adequately Treated with Once-a-month paliperidone palmitate extended-release injectable suspension (PP1M)*

| If the Last Dose | Initiate INVEGA HAFYERA |
|---|---|
| of PP1M is: | at the Following Dose: |
| 156 mg | 1,092 mg |
| 234 mg | 1,560 mg |

*Switching from the PP1M 39 mg, 78 mg and 117 mg doses was not studied.

INVEGA HAFYERA Doses for Adults Adequately Treated with Every-three-month paliperidone palmitate injectable suspension (PP3M)*

| If the Last Dose | Initiate INVEGA HAFYERA |
|---|---|
| of PP3M is: | at the Following Dose: |
| 546 mg | 1,092 mg |
| 819 mg | 1,560 mg |

*Switching from the PP3M 273 mg and 410 mg doses was not studied.
   Missed Doses: Refer to the Full Prescribing Information. (2.3)
   See Full Prescribing Information for important preparation and administration information. (2.4)

### Dosage Forms and Strengths

Extended-release injectable suspension: 1,092 mg/3.5 mL or 1,560 mg/5 mL single-dose prefilled syringes. (3)

### Contraindications

Known hypersensitivity to paliperidone, risperidone, or to any excipients in INVEGA HAFYERA. (4)

### Warnings And Precautions

Cerebrovascular Adverse Reactions in Elderly Patients with Dementia-Related Psychosis: Increased incidence of cerebrovascular adverse reactions (e.g., stroke, transient ischemic attack, including fatalities). (5.2)
Neuroleptic Malignant Syndrome: Manage with immediate discontinuation of drug and dose monitoring. (5.3)
QT Prolongation: Avoid use with drugs that also increase QT interval and in patients with risk factors for prolonged QT interval. (5.4)
Tardive Dyskinesia: Discontinue treatment if clinically appropriate (5.5)
Metabolic Changes: Monitor for hyperglycemia/diabetes mellitus, dyslipidemia, and weight gain. (5.6)
Orthostatic Hypotension and Syncope: Use with caution in patients with known cardiovascular or cerebrovascular disease and patients predisposed to hypotension. (5.7)
Leukopenia, Neutropenia, and Agranulocytosis: Perform complete blood counts (CBC) in patients with pre-existing low white blood cell count (WBC) or a history of leukopenia or neutropenia. Consider discontinuing INVEGA HAFYERA if a clinically significant decline in WBC occurs in the absence of other causative factors. (5.9)
Hyperprolactinemia: Prolactin elevations occur and persist during chronic administration. (5.10)
Potential for Cognitive and Motor Impairment: Use caution when operating machinery. (5.11)
Seizures: Use cautiously in patients with a history of seizures or with conditions that lower the seizure threshold. (5.12)

### Adverse Reactions

The most common adverse reactions were upper respiratory tract infection, injection site reaction, weight increased, headache, and parkinsonism. (6.1)

To report SUSPECTED ADVERSE REACTIONS, contact Janssen Pharmaceuticals, Inc. at 1-800-JANSSEN (1-800-526-7736) or FDA at 1-800-FDA-1088 or www.fda.gov/medwatch.

### Drug Interactions

Strong CYP3A4/P-glycoprotein (P-gp) inducers: Avoid using strong CYP3A4 and/or P-gp inducers during a dosing interval for INVEGA HAFYERA. If administering a strong inducer is necessary, consider managing the patient using paliperidone extended-release tablets. (7.1, 12.3)

### Use in Specific Populations

Pregnancy: May cause extrapyramidal and/or withdrawal symptoms in neonates with third trimester exposure. (8.1)
Renal Impairment: Not recommended. (8.6)

See 17 for PATIENT COUNSELING INFORMATION and FDA-approved patient labeling. Revised: August 2021

### Full Prescribing Information: Contents*

WARNING: INCREASED MORTALITY IN ELDERLY PATIENTS WITH DEMENTIA-RELATED PSYCHOSIS

1 INDICATIONS AND USAGE

2 DOSAGE AND ADMINISTRATION

2.1 Important Dosage and Administration Information

2.2 Recommended Dosage for INVEGA HAFYERA

2.3 Missed Doses

2.4 Instructions for Preparation and Administration

3 DOSAGE FORMS AND STRENGTHS

4 CONTRAINDICATIONS

5 WARNINGS AND PRECAUTIONS

5.1 Increased Mortality in Elderly Patient with Dementia-Related Psychosis

5.2 Cerebrovascular Adverse Reactions, Including Stroke, in Elderly Patients with Dementia-Related Psychosis

5.3 Neuroleptic Malignant Syndrome

5.4 QT Prolongation

5.5 Tardive Dyskinesia

5.6 Metabolic Changes

5.7 Orthostatic Hypotension and Syncope

5.8 Falls

5.9 Leukopenia, Neutropenia, and Agranulocytosis

5.10 Hyperprolactinemia

5.11 Potential for Cognitive and Motor Impairment

5.12 Seizures

5.13 Dysphagia

5.14 Priapism

5.15 Disruption of Body Temperature Regulation

6 ADVERSE REACTIONS

6.1 Clinical Trials Experience

6.2 Postmarketing Experience

7 DRUG INTERACTIONS

7.1 Drugs Having Clinically Important Interactions with INVEGA HAFYERA

7.2 Drugs Having No Clinically Important Interactions with INVEGA HAFYERA

8 USE IN SPECIFIC POPULATIONS

8.1 Pregnancy

8.2 Lactation

8.3 Females and Males of Reproductive Potential

8.4 Pediatric Use

8.5 Geriatric Use

8.6 Renal Impairment

8.7 Hepatic Impairment

8.8 Patients with Parkinson's Disease or Lewy Body Dementia

9 DRUG ABUSE AND DEPENDENCE

9.1 Controlled Substance

9.2 Abuse

9.3 Dependence

10 OVERDOSAGE

11 DESCRIPTION

12 CLINICAL PHARMACOLOGY

12.1 Mechanism of Action

12.2 Pharmacodynamics

12.3 Pharmacokinetics

13 NONCLINICAL TOXICOLOGY

13.1 Carcinogenesis, Mutagenesis, Impairment of Fertility

13.2 Animal Toxicology and/or Pharmacology

14 CLINICAL STUDIES

16 HOW SUPPLIED/STORAGE AND HANDLING

17 PATIENT COUNSEUNG INFORMATION

*Sections or subsections omitted Mom the full prescribing information are not listed.

### Full Prescribing Information

Warning: Increased Mortality in Elderly Patients with Dementia-Related Psychosis

Elderly patients with dementia-related psychosis treated with antipsychotic drugs are at an increased risk of death. INVEGA HAFYERA is not approved for use in patients with dementia-related psychosis [see Warnings and Precautions (5.1)].

1 Indications and Usage

INVEGA HAFYERA, an every-six-month injection is indicated for the treatment of schizophrenia in adults after they have been adequately treated with:
A once-a-month paliperidone palmitate extended-release injectable suspension (e.g., INVEGA SUSTENNA) for at least four months, or
An every-three-month paliperidone palmitate extended-release injectable suspension (e.g., INVEGA TRINZA) for at least one three-month cycle.

2 Dosage and Administration

2.1 Important Dosage and Administration Information
INVEGA HAFYERA must be administered as a gluteal intramuscular injection by a healthcare professional once every 6 months. Do not administer by any other route [see Dosage and Administration (2.4, 2.5)].
Initiate INVEGA HAFYERA only after adequate treatment has been established with either:
   A once-a-month paliperidone palmitate extended-release injectable suspension (e.g., INVEGA SUSTENNA), referred to as PP1M, once monthly for at least four months; or
   An every-three-month paliperidone palmitate extended-release injectable suspension (e.g., INVEGA TRINZA), referred to as PP3M, once every three months for at least one three-month injection cycle.
See Prescribing Information of the PP1M and PP3M products for the recommended dosage of these products.

2.2 Recommended Dosage for INVEGA HAFYERA

Switching to INVEGA HAFYERA from a PP1M Product

The recommended initial INVEGA HAFYERA dose is based on the previous PP1M dose (see Table 16). Initiate INVEGA HAFYERA when the next PP1M dose is scheduled. INVEGA HAFYERA may be administered up to 1 week before or 1 week after the next scheduled PP1M dose. When switching from PP1M to INVEGA HAFYERA, the two injection cycles immediately preceding the switch should be the same dosage strength before starting INVEGA HAFYERA.

TABLE 16
Initial INVEGA HAFYERA Dose for Adult
Patients Switching from a PP1M* Product

| Last Dose of PP1M** | Initial Dose of NVEGA HAFYERA |
|---|---|
| 156 mg | 1,092 mg |
| 234 mg | 1,560 mg |

*PPIM: Once-a-month paliperidone palmitate extended-release injectable suspension
**There are no equivalent doses of INVEGA HAFYERA for 39 mg, 78 mg, or 117 mg doses of a PP1M product, which were not studied [see Clinical Studies (14)].

Switching to INVEGA HAFYERA from a PP3M Product

The recommended initial INVEGA HAFYERA dose is based on the previous PP3M dose (see Table 17). Initiate INVEGA HAFYERA when the next PP3M dose is scheduled. INVEGA HAFYERA may be administered up to 2 weeks before or 2 weeks after the next scheduled PP3M dose.

TABLE 17
Initial NVEGA HAFYERA Dose for Adult Patients
Switching from a PP3M* Product

| Last Dose of PP3M** | Initial Dose of INVEGA HAFYERA |
|---|---|
| 546 mg | 1,092 mg |
| 819 mg | 1,560 mg |

*PP3M: Every-three-month paliperidone palmitate extended-release injectable suspension
**There are no equivalent doses of INVEGA HAFYERA for the 273 mg or 410 mg doses of a PP3M product, which were not studied [see Clinical Studies (14)].

Dosing Interval and Dosage Adjustments of INVEGA HAFYERA

Following the initial dose, administer INVEGA HAFYERA once every 6 months.

If needed, dosage adjustment can be made every 6 months between the dose of 1,092 mg to 1,560 mg based on individual response and tolerability. Because of the potential longer duration of INVEGA HAFYERA, the patient's response to an adjusted dose may not be apparent for several months [see Clinical Pharmacology (12.3)].

2.3 Missed Doses

Dosing Window

To avoid a missed dose, patients may be given the injection up to 2 weeks before or 3 weeks after the scheduled 6-month dose.

Missed Dose

If a dose of INVEGA HAFYERA is missed, re-initiate with a PP1M product using the re-initiation regimens described in Tables 3 and 4.

More than 6 Months and 3 Weeks, up to but Less than 8 Months Since Last Dose

If more than 6 months and 3 weeks but less than 8 months have elapsed since the last dose of INVEGA HAFYERA, do not administer the next dose of INVEGA HAFYERA. Instead, use the re-initiation regimen shown in Table 18:

TABLE 18
Re-initiation Regimen for Missed Dose (more than 6 months
and 3 weeks, but less than 8 months since last dose)

| | Administer | Administer |
|---|---|---|
| | PP1M Product* | INVEGA HAFYERA |
| Last Dose of | into deltoid muscle | into gluteal muscle |
| INVEGA HAFYERA | Day 1 | 1 month after Day 1 |
| 1,092 mg | 156 mg | 1,092 mg |
| 1,560 mg | 234 mg | 1,560 mg |

*PP1M: Once-a-month paliperidone palmitate extended-release injectable suspension

8 Months Up to and Including 11 Months Since Last Dose

If 8 months but up to and including 11 months have elapsed since the last dose of INVEGA HAFYERA, do not administer the next dose of INVEGA HAFYERA. Instead, use the re-initiation regimen shown in Table 19:

TABLE 19
Re-initiation Regimen for Missed Dose (8 months
up to and including 11 months since last dose)

| | Administer | | Administer |
|---|---|---|---|
| | PP1M Product* | | INVEGA HAFYERA |
| Last dose of | into deltoid muscle | | into gluteal muscle |
| INVEGA HAFYERA | Day 1 | Day 8 | 1 month after Day 8 |
| 1,092 mg | 156 mg | 156 mg | 1,092 mg |
| 1,560 mg | 156 mg | 156 mg | 1,560 mg |

*PP1M: Once-a-month paliperidone palmitate extended-release injectable suspension

More than 11 Months Since Last Dose

If more than 11 months have elapsed since the last dose of INVEGA HAFYERA, re-initiate treatment with a PP1M product as described in the prescribing information for that product. INVEGA HAFYERA can then be resumed after the patient has been adequately treated with a PP1M product for at least 4 months.

2.4 Instructions for Preparation and Administration
To be prepared and administered by a healthcare provider only.
Read the instructions for preparation and administration below and consider referring to the separate Healthcare Provider “Instructions for Use” for preparation and administration considerations.
For gluteal intramuscular injection only. Do not inject by any other route. As a universal precaution, always wear gloves.
Inspect INVEGA HAFYERA for particulate matter and discoloration prior to administration.
Do not mix with any other product or diluent.
After shaking, INVEGA HAFYERA should appear uniform, thick and milky white.
Do not use needles from the PPIM or PP3M products or other commercially-available needles to reduce the risk of blockage.
Avoid inadvertent injection into a blood vessel. Administer the dose in a single injection; do not administer the dose in divided injections. Inject slowly, deep into the upper-outer quadrant of the gluteal muscle. Future injections should be alternated between the two gluteal muscles.

Incomplete Administration
Proper shaking can reduce the likelihood for an incomplete injection. Storing the carton in a horizontal orientation improves the ability to resuspend this highly concentrated product [see How Supplied/Storage and Handling (16)].
Follow the full instructions for preparation and administration to avoid an incomplete injection.
In the event of an incompletely administered dose, do not re-inject the dose remaining in the syringe and do not administer another dose of INVEGA HAFYERA.
Closely monitor and treat the patient with oral paliperidone supplementation as clinically appropriate until the next scheduled 6-month injection of INVEGA HAFYERA. See Prescribing Information of the oral paliperidone product for the recommended dosage of these products.

Administer Every 6 Months

INVEGA HAFYERA™ (paliperidone palmitate)

Shake syringe with the syringe tip cap pointing up VERY FAST for at least 15 seconds, rest briefly, then shake again for 15 seconds.

For Gluteal Intramuscular injection only.

Preparation

INVEGA HAFYERA requires longer and faster shaking than once-a-month paliperidone palmitate extended-release injectable suspension (e.g., INVEGA SUSTENNA). INVEGA HAFYERA must be administered by a healthcare professional as a single injection. Do not divide dose into multiple injections.

INVEGA HAFYERA is intended for gluteal intramuscular use only. Inject slowly, deep into the muscle taking care to avoid injection into a blood vessel.

Dosing

Administer INVEGA HAFYERA once every 6 months.

Thin Wall Safety Needle

Thin wall safety needle is designed to be used with INVEGA HAFYERA. Therefore, it is important to only use the needle provided in the INVEGA HAFYERA suspension kit.

Dose Pack Contents:

Prefilled Syringe

Thin Wall Safety Needle, 20 G×1½″.

Only use the needle included in this kit

1. Prepare for the Injection: this Highly Concentrated Product Requires Specific Steps to Resuspend

Hold syringe with the tip cap pointing up

Shake syringe VERY FAST for at least 15 seconds, rest briefly, then shake again for 15 seconds. To ensure complete resuspension shake syringe with:
Short, VERY FAST up and down motion
Loose wrist

If more than 5 minutes pass before injection, shake the syringe VERY FAST with the tip cap pointing up again for at least 30 seconds to resuspend the Invega Hafyera. Proceed to the next step immediately after shaking.

Check Suspension for Solid Product

Mixed Well
Uniform, thick and milky white
It is normal to see air bubbles

Not Mixed Well
Solid product on sides and top of syringe
Uneven mix
Thin liquid

Product May Clog.

Shake syringe with the syringe tip cap pointing up VERY FAST for at least 15 seconds, rest, then shake again for 15 seconds.

Open Needle Pouch

Peel off the pouch cover. Place pouch with the needle inside on a clean surface.

Remove Syringe Tip Cap and Attach Needle

Hold the syringe with the tip cap pointing up. Twist and pull off the cap. Hold the syringe by the luer connection. Twist it into the safety needle with a gentle clockwise twisting motion.

Only use the needle included in this kit.

Pull Back Plunger

Hold the syringe upright. Gently pull back the plunger to clear the syringe tip of any solid product. This will make pressing the plunger easier during the injection.

Remove Air Bubbles

Press the plunger carefully until a drop of liquid comes out of the needle tip.

2. Slowly Inject Entire Content and Confirm

Select and clean a gluteal injection site. Wipe the gluteal site with an alcohol swab and allow it to dry. Do not touch, fan or blow on the injection site after you have cleaned it.

Remove Needle Sheath

Pull the needle sheath away from the needle in a straight motion. Do not twist the sheath, as this may loosen the needle from the syringe.

Slowly Inject And Confirm

Use slow, firm, consistent pressure to press the plunger completely. This should take approximately 30 seconds. Continue to press the plunger if you feel resistance. This is normal. While the needle is in the gluteal muscle, confirm that the entire content of the syringe has been injected.

Remove needle from the muscle.

3. After the Injection

Secure Needle

After the injection is complete, use your thumb or a flat surface to secure the needle in the safety device. The needle is secure when you hear a “click” sound.

Dispose of Properly and Check Injection Site

Dispose of the syringe in an approved sharps container. There may be a small amount of blood or liquid at the injection site. Hold pressure over the skin with a cotton ball or gauze pad until any bleeding stops. Do not rub the injection site. If needed, cover injection site with a bandage.

3 Dosage Forms and Strengths

INVEGA HAFYERA is a white to off-white aqueous extended-release injectable suspension for gluteal intramuscular injection in dose strengths of 1,092 mg/3.5 mL and 1,560 mg/5 mL paliperidone palmitate in single-dose prefilled syringes.

4 Contraindications

INVEGA HAFYERA is contraindicated in patients with a known hypersensitivity to either paliperidone or risperidone, or to any of the excipients in the INVEGA HAFYERA formulation. Hypersensitivity reactions, including anaphylactic reactions and angioedema, have been reported in patients treated with risperidone and in patients treated with paliperidone. Paliperidone palmitate is converted to paliperidone, which is a metabolite of risperidone.

5 Warnings and Precautions

5.1 Increased Mortality in Elderly Patients with Dementia-Related Psychosis

Elderly patients with dementia-related psychosis treated with antipsychotic drugs are at an increased risk of death. Analyses of 17 placebo-controlled trials (modal duration of 10 weeks), largely in patients taking atypical antipsychotic drugs, revealed a risk of death in drug-treated patients of between 1.6 to 1.7 times the risk of death in placebo-treated patients. Over the course of a typical 10-week controlled trial, the rate of death in drug-treated patients was about 4.5%, compared to a rate of about 2.6% in the placebo group. Although the causes of death were varied, most of the deaths appeared to be either cardiovascular (e.g., heart failure, sudden death) or infectious (e.g., pneumonia) in nature. Observational studies suggest that, similar to atypical antipsychotic drugs, treatment with conventional antipsychotic drugs may increase mortality. The extent to which the findings of increased mortality in observational studies may be attributed to the antipsychotic drug as opposed to some characteristic(s) of the patients is not clear. INVEGA HAFYERA is not approved for the treatment of patients with dementia-related psychosis [see Boxed Warning and Warnings and Precautions (5.2)].

5.2 Cerebrovascular Adverse Reactions, Including Stroke, in Elderly Patients with Dementia-Related Psychosis

In placebo-controlled trials with risperidone, aripiprazole, and olanzapine in elderly subjects with dementia, there was a higher incidence of cerebrovascular adverse reactions (cerebrovascular accidents and transient ischemic attacks) including fatalities compared to placebo-treated subjects. No studies have been conducted with oral paliperidone, the 1-month paliperidone palmitate extended-release injectable suspension, the 3-month paliperidone extended-release injectable suspension or INVEGA HAFYERA in elderly patients with dementia. These medications are not approved for the treatment of patients with dementia-related psychosis [see Boxed Warning and Warnings and Precautions (5.1)].

5.3 Neuroleptic Malignant Syndrome

Neuroleptic Malignant Syndrome (NMS), a potentially fatal symptom complex, has been reported in association with antipsychotic drugs, including paliperidone.

Clinical manifestations of NMS are hyperpyrexia, muscle rigidity, altered mental status, including delirium, and autonomic instability (irregular pulse or blood pressure, tachycardia, diaphoresis, and cardiac dysrhythmia). Additional signs may include elevated creatine phosphokinase, myoglobinuria (rhabdomyolysis), and acute renal failure.

If NMS is suspected, discontinue INVEGA HAFYERA and provide symptomatic treatment and monitoring.

5.4 QT Prolongation

Paliperidone causes a modest increase in the corrected QT (QTc) interval. The use of paliperidone should be avoided in combination with other drugs that are known to prolong QTc including Class 1A (e.g., quinidine, procainamide) or Class III (e.g., amiodarone, sotalol) antiarrhythmic medications, antipsychotic medications (e.g., chlorpromazine, thioridazine), antibiotics (e.g., gatifloxacin, moxifloxacin), or any other class of medications known to prolong the QTc interval. Paliperidone should also be avoided in patients with congenital long QT syndrome and in patients with a history of cardiac arrhythmias.

Certain circumstances may increase the risk of the occurrence of Torsades de pointes and/or sudden death in association with the use of drugs that prolong the QTc interval, including (1) bradycardia; (2) hypokalemia or hypomagnesemia; (3) concomitant use of other drugs that prolong the QTc interval; and (4) presence of congenital prolongation of the QT interval.

The effects of paliperidone on the QT interval were evaluated in a double-blind, active-controlled (moxifloxacin 400 mg single dose), multicenter Thorough QT study with oral paliperidone in adult patients, and in four fixed-dose efficacy studies and one maintenance study of the 1-month paliperidone palmitate injectable product.

In the Thorough QT study (n=141), the 8 mg dose of immediate-release oral paliperidone (n=50) showed a mean placebo-subtracted increase from baseline in QTcLD (QT interval corrected for heart rate using the population specified linear derived method) of 12.3 msec (90% CI: 8.9; 15.6) on day 8 at 1.5 hours post-dose. The mean steady-state peak plasma concentration for this 8 mg dose of paliperidone immediate release (C_{max ss}=113 ng/mL) was approximately 1.3-fold the exposure with the maximum recommended 1,560 mg dose of INVEGA HAFYERA administered in the gluteal muscle (mean C_{max md}=89.3 ng/mL). In this same study, a 4 mg dose of the immediate-release oral formulation of paliperidone, for which C_{max ss}=35 ng/mL, showed an increased placebo-subtracted QTcLD of 6.8 msec (90% CI: 3.6; 10.1) on day 2 at 1.5 hours post-dose.

In the four fixed-dose efficacy studies of the 1-month paliperidone palmitate injectable product, no subject had a change in QTcLD exceeding 60 msec and no subject had a QTcLD value of >500 msec at any time point. In the maintenance study, no subject had a QTcLD change >60 msec, and one subject had a QTcLD value of 507 msec (Bazett's QT corrected interval [QTcB] value of 483 msec); this latter subject also had a heart rate of 45 beats per minute.

In the INVEGA HAFYERA randomized double-blind active controlled study in subjects with schizophrenia, during the double-blind Phase, QTcLD exceeding 60 msec was observed in 2 subjects (0.4%) in the INVEGA HAFYERA treatment group and in 2 subjects (0.9%) in the PP3M treatment group. No subject had a QTcLD value of >480 msec at any point in the study.

5.5 Tardive Dyskinesia

Tardive dyskinesia, a syndrome of potentially irreversible, involuntary, dyskinetic movements, may develop in patients treated with antipsychotic drugs. Although the prevalence of the syndrome appears to be highest among the elderly, especially elderly women, it is impossible to predict which patients will develop the syndrome. Whether antipsychotic drug products differ in their potential to cause tardive dyskinesia is unknown.

The risk of developing tardive dyskinesia and the likelihood that it will become irreversible appear to increase as the duration of treatment and the total cumulative dose. The syndrome can develop after relatively brief treatment periods, even at low doses. It may also occur after discontinuation of treatment.

Tardive dyskinesia may remit, partially or completely, if antipsychotic treatment is discontinued. Antipsychotic treatment itself may suppress (or partially suppress) the signs and symptoms of the syndrome and may thus mask the underlying process. The effect of symptomatic suppression on the long-term course of the syndrome is unknown.

Given these considerations, INVEGA HAFYERA should be prescribed in a manner that is most likely to minimize the occurrence of tardive dyskinesia. Chronic antipsychotic treatment should generally be reserved for patients who suffer from a chronic illness that is known to respond to antipsychotic drugs. In patients who do require chronic treatment, the smallest dose and the shortest duration of treatment producing a satisfactory clinical response should be sought. The need for continued treatment should be reassessed periodically.

If signs and symptoms of tardive dyskinesia appear in a patient treated with INVEGA HAFYERA, drug discontinuation should be considered. Consideration should be given to the long-acting nature of INVEGA HAFYERA. However, some patients may require treatment with INVEGA HAFYERA despite the presence of the syndrome.

5.6 Metabolic Changes

Atypical antipsychotic drugs have been associated with metabolic changes that may increase cardiovascular/cerebrovascular risk. These metabolic changes include hyperglycemia, dyslipidemia, and body weight gain. While all of the drugs in the class have been shown to produce some metabolic changes, each drug has its own specific risk profile.

Hyperglycemia and Diabetes Mellitus

Hyperglycemia and diabetes mellitus, in some cases extreme and associated with ketoacidosis or hyperosmolar coma or death, have been reported in patients treated with all atypical antipsychotics. These cases were, for the most part, seen in post-marketing clinical use and epidemiologic studies, not in clinical trials. Hyperglycemia and diabetes have been reported in trial subjects treated with INVEGA HAFYERA. Assessment of the relationship between atypical antipsychotic use and glucose abnormalities is complicated by the possibility of an increased background risk of diabetes mellitus in patients with schizophrenia and the increasing incidence of diabetes mellitus in the general population. Given these confounders, the relationship between atypical antipsychotic use and hyperglycemia-related adverse events is not completely understood. However, epidemiological studies suggest an increased risk of hyperglycemia-related adverse reactions in patients treated with the atypical antipsychotics.

Patients with an established diagnosis of diabetes mellitus who are started on atypical antipsychotics should be monitored regularly for worsening of glucose control. Patients with risk factors for diabetes mellitus (e.g., obesity, family history of diabetes) who are starting treatment with atypical antipsychotics should undergo fasting blood glucose testing at the beginning of treatment and periodically during treatment. Any patient treated with atypical antipsychotics should be monitored for symptoms of hyperglycemia including polydipsia, polyuria, polyphagia, and weakness. Patients who develop symptoms of hyperglycemia during treatment with atypical antipsychotics should undergo fasting blood glucose testing. In some cases, hyperglycemia has resolved when the atypical antipsychotic was discontinued; however, some patients required continuation of anti-diabetic treatment despite discontinuation of the suspect drug.

Data from the randomized double-blind active controlled study with INVEGA HAFYERA in patients with schizophrenia are presented in Table 20.

TABLE 20
Change in Fasting Glucose from the randomized
double-blind active controlled study with
INVEGA HAFYERA in patients with schizophrenia

| | PP3M¹ | INVEGA HAFYERA |
|---|---|---|
| Total no. of patients^{a} | N = 195 | N = 423 |
| Normal to high | 3% | 4% |
| Impaired glucose tolerance to high | 4% | 5% |
| Normal/impaired glucose tolerance | 7% | 9% |
| to high | | |
| <126 mg/dL to >=140 mg/dL | 4% | 5% |
| <126 mg/dL to >=200 mg/dL | 0 | 1% |
| <126 mg/dL to >=300 mg/dL | 0 | <1% |

¹PP3M—Every-three-month paliperidone palmitate extended-release injectable suspension
^{a}The number of subjects with paired fasting data (baseline and any post baseline assessment).
Using the conversion factor (1 mg/dL = 0.05551 mmol/L) the ADA specified limits are as follows:
Normal: <100 mg/dL (<5.551 mmol/L)
Impaired: ≥100 mg/dL (≥5.551 mmol/L) to <126 mg/dL (<6.994 mmol/L)
High: ≥126 mg/dL (≥6.994 mmol/L)
126 mg/dL = 6.994 mmol/L; 140 mg/dL = 7.771 mmol/L; 200 mg/dL = 11.102 mmol/L; 300 mg/dL = 16.653 mmol/L

Dyslipidemia

Undesirable alterations in lipids have been observed in patients treated with atypical antipsychotics.

Shifts in lipid parameters from the randomized double-blind active controlled study with INVEGA HAFYERA in patients with schizophrenia are presented in Table 21.

TABLE 21
Shifts in Fasting Lipids in the Double-Blind Phase
from the randomized active controlled study with
INVEGA HAFYERA in patients with schizophrenia

| | PP3M¹ | | INVEGA HAFYERA | |
|---|---|---|---|---|
| | N = 194 | | N = 423 | |
| Fasting Cholesterol (mg/dL) | 2 | (1%) | 3 | (0.7%) |
| <200 mg/dL to >=240 mg dL | | | | |
| Fasting HDL Cholesterol (mg di) | 28 | (14%) | 55 | (13%) |
| >=40 mg/dL to <40 mg/dL | | | | |
| Fasting LDL Cholesterol (mg/dL) | 1 | (0.5%) | 2 | (0.5%) |
| <100 mg/dL to >=160 mg/dL | | | | |
| Fasting Triglycerides (mg/dL) | 22 | (11%) | 22 | (5%) |
| <150 mg/dL to >−=200 mg/dL | | | | |

¹PP3M—Every-three-month paliperidone palmitate extended-release injectable suspension.
For each fasting parameter, subjects with both Baseline (DB) record and any post baseline (DB) record during Double-Blind Phase are included in the denominator.

Change in Body Weight

Weight gain has been observed with atypical antipsychotic use. Clinical monitoring of weight is recommended. In the randomized active controlled clinical study of INVEGA HAFYERA, the overall mean weight change during the double-blind Phase was similar to PP3M.

5.7 Orthostatic Hypotension and Syncope

Paliperidone can induce orthostatic hypotension and syncope in some patients because of its alpha-adrenergic blocking activity.

Use INVEGA HAFYERA with caution in patients with known cardiovascular disease (e.g., heart failure, history of myocardial infarction or ischemia, conduction abnormalities), cerebrovascular disease, or conditions that predispose the patient to hypotension (e.g., dehydration, hypovolemia, and treatment with antihypertensive medications). Monitoring of orthostatic vital signs should be considered in patients who are vulnerable to hypotension.

5.8 Falls

Somnolence, postural hypotension, motor and sensory instability have been reported with the use of antipsychotics, including paliperidone palmitate, which may lead to falls and, consequently, fractures or other fall-related injuries. For patients, particularly the elderly, with diseases, conditions, or medications that could exacerbate these effects, assess the risk of falls when initiating antipsychotic treatment and recurrently for patients on long-term antipsychotic therapy.

5.9 Leukopenia, Neutropenia, and Agranulocytosis

In clinical trial and/or postmarketing experience, events of leukopenia and neutropenia have been reported temporally related to antipsychotic agents, including INVEGA HAFYERA. Agranulocytosis has also been reported.

Possible risk factors for leukopenia/neutropenia include pre-existing low white blood cell count (WBC)/absolute neutrophil count (ANC) and history of drug-induced leukopenia/neutropenia. In patients with a history of a clinically significant low WBC/ANC or a drug-induced leukopenia/neutropenia, perform a complete blood count (CBC) frequently during the first few months of therapy. In such patients, consider discontinuation of INVEGA HAFYERA at the first sign of a clinically significant decline in WBC in the absence of other causative factors.

Monitor patients with clinically significant neutropenia for fever or other symptoms or signs of infection and treated promptly if such symptoms or signs occur. Discontinue INVEGA HAFYERA in patients with severe neutropenia (absolute neutrophil count <1000/mm³) and follow their WBC until recovery.

5.10 Hyperprolactinemia

Like other drugs that antagonize dopamine D₂ receptors, paliperidone elevates prolactin levels and the elevation persists during chronic administration. Paliperidone has a prolactin-elevating effect similar to that seen with risperidone, a drug that is associated with higher levels of prolactin than other antipsychotic drugs.

Hyperprolactinemia, regardless of etiology, may suppress hypothalamic GnRH, resulting in reduced pituitary gonadotrophin secretion. This, in turn, may inhibit reproductive function by impairing gonadal steroidogenesis in both female and male patients. Galactorrhea, amenorrhea, gynecomastia, and impotence have been reported in patients receiving prolactin-elevating compounds. Long-standing hyperprolactinemia when associated with hypogonadism may lead to decreased bone density in both female and male subjects.

Tissue culture experiments indicate that approximately one-third of human breast cancers are prolactin dependent in vitro, a factor of potential importance if the prescription of these drugs is considered in a patient with previously detected breast cancer. An increase in the incidence of pituitary gland, mammary gland, and pancreatic islet cell neoplasia (mammary adenocarcinomas, pituitary and pancreatic adenomas) was observed in the risperidone carcinogenicity studies conducted in mice and rats [see Nonclinical Toxicology (13.1)]. Neither clinical studies nor epidemiologic studies conducted to date have shown an association between chronic administration of this class of drugs and tumorigenesis in humans, but the available evidence is too limited to be conclusive.

Median prolactin levels remained relatively stable throughout the open-label and double-blind phases in male subjects, whereas in female subjects, median prolactin levels increased. During the double-blind phase, median prolactin levels continued to increase after dosing in both the INVEGA HAFYERA and PP3M groups, returning to baseline level at Month 6 and at Month 12 (end of double-blind phase).

During the double-blind phase, prolactin levels relative to reference range (>13.13 ng/mL in males and >26.72 ng/mL in females) from maintenance baseline were noted in a similar percentage of subjects in the INVEGA HAFYERA and PP3M groups in both males (35% vs 36%) and females (29% vs. 30%). In the INVEGA HAFYERA group, 14 females (2.9%) and 4 males (0.8%) experienced potentially prolactin-related adverse reactions, while 6 females (2.7%) and 1 male (0.4%) in the PP3M experienced potentially prolactin-related adverse reactions.

5.11 Potential for Cognitive and Motor Impairment

Somnolence and sedation were reported as adverse reactions in patients treated with INVEGA HAFYERA [see Adverse Reactions (6.1)]. Antipsychotics, including INVEGA HAFYERA, have the potential to impair judgment, thinking, or motor skills. Patients should be cautioned about performing activities requiring mental alertness, such as operating hazardous machinery or operating a motor vehicle, until they are reasonably certain that paliperidone therapy does not adversely affect them.

5.12 Seizures

In the 6-month paliperidone palmitate extended-release injectable suspension double-blind active controlled trial there were no reports of seizures or convulsions, nor were any reports made in the long-term maintenance trial of PP3M. In the pivotal clinical studies with PP1M which included four fixed-dose, double-blind, placebo-controlled studies in subjects with schizophrenia, <1% (1/1293) of subjects treated with the PP1M experienced an adverse event of convulsion compared with <1% (1/510) of placebo-treated subjects who experienced an adverse event of grand mal convulsion.

Like other antipsychotic drugs, INVEGA HAFYERA should be used cautiously in patients with a history of seizures or other conditions that potentially lower the seizure threshold. Conditions that lower the seizure threshold may be more prevalent in patients 65 years or older.

5.13 Dysphagia

Esophageal dysmotility and aspiration have been associated with antipsychotic drug use. INVEGA HAFYERA and other antipsychotic drugs should be used cautiously in patients at risk for aspiration pneumonia.

5.14 Priapism

A case (0.2%) of priapism was reported in the clinical trial with INVEGA HAFYERA. Priapism has been reported with oral paliperidone during postmarketing surveillance. Drugs with alpha-adrenergic blocking effects have been reported to induce priapism. Severe priapism may require surgical intervention.

5.15 Disruption of Body Temperature Regulation

Disruption of the body's ability to reduce core body temperature has been attributed to antipsychotic agents. Appropriate care is advised when prescribing INVEGA HAFYERA to patients who will be experiencing conditions which may contribute to an elevation in core body temperature, e.g., exercising strenuously, exposure to extreme heat, receiving concomitant medication with anticholinergic activity, or being subject to dehydration.

6 Adverse Reactions

The following are discussed in more detail in other sections of the labeling:
Increased mortality in elderly patients with dementia-related psychosis [see Boxed Warning and Warnings and Precautions (5.1)]
Cerebrovascular adverse reactions, including stroke, in elderly patients with dementia-related psychosis [see Warnings and Precautions (5.2)]
Neuroleptic malignant syndrome [see Warnings and Precautions (5.3)]
QT prolongation [see Warnings and Precautions (5.4)]
Tardive dyskinesia [see Warnings and Precautions (5.5)]
Metabolic changes [see Warnings and Precautions (5.6)]
Orthostatic hypotension and syncope [see Warnings and Precautions (5.7)]
Falls [see Warnings and Precautions (5.8)]
Leukopenia, neutropenia, and agranulocytosis [see Warnings and Precautions (5.9)]
Hyperprolactinemia [see Warnings and Precautions (5.10)]
Potential for cognitive and motor impairment [see Warnings and Precautions (5.11)]
Seizures [see Warnings and Precautions (5.12)]
Dysphagia [see Warnings and Precautions (5.13)]
Priapism [see Warnings and Precautions (5.14)]
Disruption of body temperature regulation [see Warnings and Precautions (5.15)]

6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in clinical practice.

Patient Exposure

The data described in this section is derived from the randomized double-blind active controlled non-inferiority study of INVEGA HAFYERA and 3-month paliperidone palmitate extended-release injectable suspension. During the double-blind phase, 478 patients were randomized to receive 2 injection cycles of INVEGA HAFYERA over a 12-month duration. The mean (SD) duration of exposure was 329.8 (86.97) days in the INVEGA HAFYERA group and 336.4 (80.89) days in the PP3M group during the double-blind phase:

Adverse Reactions in the Double-Blind, Active-Controlled Clinical Trial

Commonly Observed Adverse Reactions: The most common adverse reactions (incidence at least 5% in the double-blind Phase) of the INVEGA HAFYERA clinical trial were, upper respiratory tract infection, injection site reaction, weight increased, headache and parkinsonism.

Discontinuation of Treatment Due to Adverse Reactions: In the double-blind phase of the INVEGA HAFYERA clinical trial 1.3% of subjects in the INVEGA HAFYERA group and 0.4% of subjects in the 3-month paliperidone palmitate extended-release injectable suspension group discontinued due to adverse reactions.

Adverse Reactions Occurring at an Incidence of 2% or More in INVEGA HAFYERA-Treated Patients: Table 22 lists the adverse reactions reported in the INVEGA HAFYERA clinical trial.

TABLE 22
Incidences of Adverse Reactions 2% or More of INVEGA
HAFYERA-Treated Patients for the Double-Blind Phase
of the Randomized Double-blind Active Controlled
Trial in Patients with Schizophrenia

| | Double Blind | |
|---|---|---|
| | PP3M¹ | INVEGA HAFYERA |
| | (N = 224) | (N = 478) |
| System Organ Class | % | % |
| Adverse Reaction | | |
| Gastrointestinal disorders | | |
| Diarrhea* | 1 | 2 |
| General disorders and | | |
| administration site conditions | | |
| Injection site reaction* | 5 | 11 |
| Infections and infestations | | |
| Upper respiratory tract infection* | 13 | 12 |
| Urinary tract infection | 1 | 3 |
| Metabolism and nutrition disorders | | |
| Weight increased | 8 | 9 |
| Musculoskeletal and | | |
| connective tissue disorders | | |
| Back pain* | 1 | 3 |
| Musculoskeletal pain* | 1 | 3 |
| Nervous system disorders | | |
| Akathisia* | 4 | 4 |
| Headache | 5 | 7 |
| Extrapyramidal symptoms* | 5 | 7 |
| Psychiatric disorders | | |
| Psychosis* | 3 | 3 |
| Anxiety | 0 | 3 |
| Insomnia* | 2 | 3 |

¹PP3M—Every-three-month paliperidone palmitate extended-release injectable suspension
*The following terms were combined:
Diarrhea includes: Diarrhea, Diarrhea infectious.
Injection site reaction: includes Injection site reaction, Injection site discomfort, Injection site erythema, injection site hemorrhage, Injection site induration, Injection site nodule, Injection site oedema, Injection site pain, Injection site swelling.
Weight increased includes: Weight increased, Body mass index increased, Obesity, Waist circumference increased.
Upper respiratory tract infection includes: Upper respiratory tract infection, Nasopharyngitis, Pharyngitis, Rhinitis, Viral pharyngitis, Viral upper respiratory tract infection.
Back pain includes: Back pain, Neck pain, Spinal pain.
Musculoskeletal pain includes: Musculoskeletal pain, Musculoskeletal chest pain, Myalgia, Pain in extremity.
Akathisia includes: Akathisia, Restless legs syndrome, Restlessness.
Extrapyramidal symptoms includes: blepharospasms, bradykinesia, drooling, dyskinesia, dystonia, hypokinesia, musculoskeletal stiffness, muscle rigidity, muscle spasms, oculogyric crisis, Parkinsonism, Parkinsonism rest tremor, reduced facial expression, tardive dyskinesia.
Insomnia includes: Insomnia, Initial insomnia, Middle insomnia.
Psychosis includes: acute psychosis, delusion, delusion of reference, hallucination (auditory), psychotic disorder, psychotic symptom, and schizophrenia.

Demographic Differences

An examination of population subgroups in the INVEGA HAFYERA trial did not reveal any evidence of differences in safety on the basis of age, gender, or race alone.

Extrapyramidal Symptoms (EPS)

Data from the randomized double-blind active controlled study provided information regarding EPS. Several methods were used to measure EPS: (1) the Simpson-Angus Rating Scale Global Score which broadly evaluates parkinsonism, (2) the Barnes Akathisia Rating Scale Global Clinical Rating Score which evaluates akathisia, (3) the Abnormal Involuntary Movement Scale scores which evaluates dyskinesia, and (4) use of anticholinergic medications to teat EPS (Table 23) and (5) incidence of spontaneous reports of EPS (Table 24).

TABLE 23
Extrapyramidal Symptoms (EPS) Assessed by Rating
Scales Incidence and Use of Anticholinergic
Medication During the Double-blind Phase

| | PP3M¹ | INVEGA HAFYERA |
|---|---|---|
| | (N = 224) | (N = 478) |
| | % | % |
| Use of Anticholinergic Medication^{(a)} | 13 | 15 |
| Parkinsonism^{(b)} | 6 | 7 |
| Akathisia^{(c)} | 3 | 3 |
| Dyskinesia^{(d)} | 1 | 1 |

¹PP3M—Every-three-month paliperidone palmitate extended-release injectable suspension
^{(a)}Use of Anti-EPS Medication During the Double-blind Phase
^{(b)}Percent of subjects with Simpson-Angus Scale Global Score >0.3 (Global Score defined as total sum of items score divided by the number of items).
^{(c)}Percent of subjects with Barnes Akathisia Rating Scale Global Clinical Rating Score ≥2
^{(d)}Percent of subjects with a score ≥3 on any of the first seven items or a score ≥2 on two or more of any of the first seven items of the Abnormal involuntary Movement Scale
Note:
Percentages are calculated based on number of subjects in the DB Safety analysis set per treatment group.

TABLE 24
Extrapyramidal Symptoms (EPS)-Related
Events by MedDRA Preferred Term

| | Double-blind Phase | |
|---|---|---|
| | PP3M¹ | INVEGA HAFYERA |
| | (N = 224) | (N = 478) |
| EPS Group | % | % |
| Overall percentage of subjects | 9 | 10 |
| with EPS-related adverse events | | |
| Parkinsonism | 4 | 5 |
| Hyperkinesia | 4 | 4 |
| Tremor | 0 | <1 |
| Dyskinesia | 1 | 2 |
| Dystonia | 1 | 1 |

¹PP3M—Every-three-month paliperidone palmitate extended-release injectable suspension
Parkinsonism group includes: Bradykinesia, drooling, hypokinesia, muscle rigidity, musculoskeletal stiffness, parkinsonism, parkinsonian rest tremor, reduced facial expression
Hyperkinesia group includes: Akathisia, restlessness, restless legs syndrome
Dyskinesia group includes: Dyskinesia, muscle twitching, tardive dyskinesia
Dystonia group includes: Blepharospasm, dystonia, muscle spasms, oculogyric crisis

Dystonia

Symptoms of dystonia, prolonged abnormal contractions of muscle groups, may occur in susceptible individuals during the first few days of treatment. Dystonic symptoms include: spasm of the neck muscles, sometimes progressing to tightness of the throat, swallowing difficulty, difficulty breathing, and/or protrusion of the tongue. While these symptoms can occur at low doses, they occur more frequently and with greater severity with high potency and at higher doses of first-generation antipsychotic drugs. An elevated risk of acute dystonia is observed in males and younger age groups.

Pain Assessment and Local Injection Site Reactions

Investigator ratings of injection site. Induration, redness and swelling were observed in 13% in the INVEGA HAFYERA group and 9% in the PP3M group during the double-blind Phase. Investigator evaluation of tenderness was higher for subjects in the INVEGA HAFYERA group versus the 3-month paliperidone palmitate extended-release injectable suspension group (31% vs. 19%) during the double-blind Phase. Active INVEGA HAFYERA medication was given at double-blind baseline and Month 6, while placebo medication was given at the other injection times.

Subject ratings of injection site pain. The average score for the subject's evaluation of injection pain on a scale of 0 to 100 was approximately 16 at the open-label Phase end point and approximately 5 in both groups at the double-blind Phase end point.

Other Adverse Reactions Observed During the Clinical Trial Evaluation of INVEGA HAFYERA

The following additional adverse reactions were identified in the randomized double-blind active controlled study. The following list does not include reactions: 1) already listed in previous tables or elsewhere in labeling, 2) for which a drug cause was remote, 3) which were so general as to be uninformative, 4) which were not considered to have significant clinical implications.

Blood and lymphatic system disorders: anemia

Cardiac disorders: bradycardia, tachycardia

Ear and labyrinth disorders: vertigo

Gastrointestinal disorders: constipation, nausea, vomiting

General disorders and administration site conditions: fatigue

Hepatobiliary disorders: transaminases increased

Infections and infestations: cystitis, respiratory tract infection, tonsillitis

Metabolism and nutritional disorders: decreased appetite, increased appetite, weight decreased

Psychiatric disorders: depression

Reproductive system and breast disorders: breast pain, menstrual disorder

Skin and subcutaneous tissue disorders: rash

Vascular disorders: hypertension

Additional Adverse Reactions Reported in Clinical Trials with the 1-Month and 3-Month Paliperidone Palmitate Extended-Release Injectable Suspension

The following is a list of additional adverse reactions that have been reported in clinical trials with the 1-month and 3-month paliperidone palmitate extended-release injectable suspensions that are not listed elsewhere:

Cardiac disorders: atrioventricular block first degree, bundle branch block, palpitations, postural orthostatic tachycardia syndrome

Eye disorders: eye movement disorder, eye rolling, oculogyric crisis, vision blurred

Gastrointestinal disorders: abdominal discomfort/abdominal pain upper, diarrhea, dry mouth, toothache

General disorders and administration site conditions: asthenia, chest discomfort

Immune system disorders: hypersensitivity

Investigations: electrocardiogram abnormal

Metabolism and nutrition disorders: hyperinsulinemia

Musculoskeletal and connective tissue disorders: myalgia, pain in extremity, joint stiffness, muscle spasms, muscle twitching, nuchal rigidity

Nervous system disorders: bradykinesia, cerebrovascular accident, convulsion, dizziness, dizziness postural, dysarthria, hypertonia, lethargy, oromandibular dystonia, psychomotor hyperactivity, syncope

Psychiatric disorders: agitation, nightmare

Reproductive system and breast disorders: breast discharge, erectile dysfunction, gynecomastia, sexual dysfunction

Respiratory, thoracic and mediastinal disorders: cough

Skin and subcutaneous tissue disorders: drug eruption, eczema, pruritus, pruritus generalized, urticaria

Vascular disorders: hypotension, orthostatic hypotension

Additional Adverse Reactions Reported in Clinical Trials with Oral Paliperidone

The following is a list of additional adverse reactions that have been reported in clinical trials with oral paliperidone:

Cardiac disorders: bundle branch block left, sinus arrhythmia

Gastrointestinal disorders: abdominal pain, constipation, flatulence, small intestinal obstruction

General disorders and administration site conditions: edema, edema peripheral

Immune system disorders: anaphylactic reaction

Musculoskeletal and connective tissue disorders: arthralgia, torticollis, trismus

Nervous system disorders: grand mal convulsion, parkinsonian gait, transient ischemic attack

Psychiatric disorders: sleep disorder

Reproductive system and breast disorders: breast engorgement, breast tenderness, retrograde ejaculation

Respiratory, thoracic and mediastinal disorders: nasal congestion, pharyngolaryngeal pain, pneumonia aspiration

Skin and subcutaneous tissue disorders: rash papular

Vascular disorders: ischemia

6.2 Postmarketing Experience

The following adverse reactions have been identified during post-approval use of paliperidone; because these reactions were reported voluntarily from a population of uncertain size, it is not always possible to reliably estimate their frequency or establish a causal relationship to drug exposure: angioedema, catatonia, ileus, somnambulism, swollen tongue, thrombotic thrombocytopenic purpura, urinary incontinence, and urinary retention.

Cases of anaphylactic reaction after injection with the 1-month paliperidone palmitate extended-release suspension have been reported during postmarketing experience in patients who have previously tolerated oral risperidone or oral paliperidone.

Paliperidone is the major active metabolite of risperidone. Adverse reactions reported with oral risperidone and risperidone long-acting injection can be found in the Adverse Reactions (6) section of the Prescribing Information for those products.

7 Drug Interactions

7.1 Drugs Having Clinically Important Interactions with INVEGA HAFYERA

Because paliperidone palmitate is hydrolyzed to paliperidone, results from studies with oral paliperidone should be taken into consideration when assessing drug-drug interaction potential. In addition, consider the 6-month dosing interval and the half-life of INVEGA HAFYERA [see Clinical Pharmacology (12.3)].

Table 25 presents clinically significant drug interactions with INVEGA HAFYERA.

TABLE 25
Clinically Important Drug Interactions with INVEGA HAFYERA
Centrally acting Drugs and Alcohol

| | |
|---|---|
| Clinical Rationale | Given the primary CNS effects of paliperidone, concomitant use of |
| | centrally acting drugs and alcohol may modulate the CNS effects |
| | of INVEGA HAFYERA. |
| Clinical Recommendation | INVEGA HAFYERA should be used with caution with other |
| | centrally acting drugs and alcohol. |

Drugs with Potential for Inducing Orthostatic Hypotension

| | |
|---|---|
| Clinical Rationale | Because INVEGA HAFYERA has the potential for inducing |
| | orthostatic hypotension, an additive effect may occur when |
| | INVEGA HAFYERA is administered with other therapeutic agents |
| | that have this potential [see Warnings and Precautions (5.7)]. |
| Clinical Recommendation | Monitor orthostatic vital signs in patients who are vulnerable to |
| | hypotension (see Warnings and Precautions (5.7)]. |

Strong Inducers of CYP3A4 and P-gp

| | |
|---|---|
| Clinical Rationale | The concomitant use of INVEGA HAFYERA and strong inducers |
| | of CYP3A4 and P-gp may decrease the exposure of paliperidone |
| | [see Clinical Pharmacology (12.3)]. |
| Clinical Recommendation | Avoid using CYP3A4 and/or P-gp inducers with INVEGA |
| | HAFYERA during the 6-month dosing interval, if possible. If |
| | administering a strong inducer is necessary, consider managing the |
| | patient using paliperidone extended-release tablets [see Dosage |
| | and Administration (2.7)]. |
| Examples | carbamazepine, rifampin, or St. John's Wort |

Levodopa and Other Dopamine Agonists

| | |
|---|---|
| Clinical Rationale | Paliperidone may antagonize the effect of levodopa and other |
| | dopamine agonists. |
| Clinical Recommendation | Monitor and manage patient as clinically appropriate. |

7.2 Drugs Having No Clinically Important Interactions with INVEGA HAFYERA

Based on pharmacokinetic studies with oral paliperidone, no dosage adjustment of INVEGA HAFYERA is required when administered concomitantly with valproate [see Clinical Pharmacology (12.3)]. Additionally, no dosage adjustment is necessary for valproate when co-administered with HAFYERA [see Clinical Pharmacology (12.3)].

Pharmacokinetic interaction between lithium and INVEGA HAFYERA is unlikely.

Paliperidone is not expected to cause clinically important pharmacokinetic interactions with drugs that are metabolized by cytochrome P450 isozymes. In vitro studies indicate that CYP2D6 and CYP3A4 may be involved in paliperidone metabolism; however, there is no evidence in vivo that inhibitors of these enzymes significantly affect the metabolism of paliperidone. Paliperidone is not a substrate of CYP1A2, CYP2A6, CYP2C9, and CYP2C19; an interaction with inhibitors or inducers of these isozymes is unlikely [see Clinical Pharmacology (12.3)].

8 Use in Specific Populations

8.1 Pregnancy

Pregnancy Exposure Registry

There is a pregnancy exposure registry that monitors pregnancy outcomes in women exposed to atypical antipsychotics, including INVEGA HAFYERA, during pregnancy. Healthcare providers are encouraged to register patients by contacting the National Pregnancy Registry for Atypical Antipsychotics at 1-866-961-2388 or online at http://womensmentalhealth.org/clinical-and-research-programs/pregnancyregistry/.

Risk Summary

Neonates exposed to antipsychotic drugs during the third trimester of pregnancy are at risk for extrapyramidal and/or withdrawal symptoms following delivery (see Clinical Considerations). Overall, available data from published epidemiologic studies of pregnant women exposed to paliperidone have not established a drug-associated risk for major birth defects, miscarriage, or adverse maternal or fetal outcomes (see Data). There are risks to the mother associated with untreated schizophrenia and with exposure to antipsychotics, including INVEGA HAFYERA during pregnancy (see Clinical Considerations). Paliperidone has been detected in plasma in adult subjects up to 18 months after a single-dose administration of 3-month paliperidone palmitate extended-release injectable suspension. [See Clinical Pharmacology (12.3)]. The clinical significance of INVEGA HAFYERA administered before pregnancy or anytime during pregnancy is not known.

The estimated background risk of major birth defects and miscarriage for the indicated population is unknown. All pregnancies have a background risk of birth defects, loss, or other adverse outcomes. In the U.S. general population, the estimated background risk of major birth defects and miscarriage in clinically recognized pregnancies is 2 to 4% and 15 to 20%, respectively.

In animal reproduction studies, there were no treatment related effects on the offspring when pregnant rats were injected intramuscularly with paliperidone palmitate or when pregnant rats and rabbits were treated orally with paliperidone during the period of organogenesis. Additional reproduction toxicity studies were conducted with orally administered risperidone, which is extensively converted to paliperidone (see Animal Data).

Clinical Considerations

Disease-Associated Maternal and/or Embryo/Fetal Risk

There is a risk to the mother from untreated schizophrenia, including increased risk of relapse, hospitalization, and suicide. Schizophrenia is associated with increased adverse perinatal outcomes, including preterm birth. It is not known if this is a direct result of the illness or other comorbid factors.

Fetal/Neonatal Adverse Reactions

Extrapyramidal and/or withdrawal symptoms, including agitation, hypertonia, hypotonia, tremor, somnolence, respiratory distress, and feeding disorder have been reported in neonates who were exposed to antipsychotic drugs, including INVEGA HAFYERA, during the third trimester of pregnancy. These symptoms have varied in severity. Monitor neonates for extrapyramidal and/or withdrawal symptoms and manage symptoms appropriately. Some neonates recovered within hours or days without specific treatment; others required prolonged hospitalization.

Data

Human Data

Published data from observational studies, birth registries, and case reports on the use of atypical antipsychotics during pregnancy do not report a clear association with antipsychotics and major birth defects. A prospective observational study including 6 women treated with risperidone, the parent compound of paliperidone, demonstrated placental passage of risperidone and paliperidone. A retrospective cohort study from a Medicaid database of 9258 women exposed to antipsychotics during pregnancy did not indicate an overall increased risk for major birth defects. There was a small increase in the risk of major birth defects (RR=1.26, 95% CI 1.02-1.56) and of cardiac malformations (RR=1.26, 95% CI 0.88-1.81) in a subgroup of 1566 women exposed to the parent compound of paliperidone, risperidone, during the first trimester of pregnancy; however, there is no mechanism of action to explain the difference in malformation rates.

Animal Data

No developmental toxicity studies were conducted with the 6-month paliperidone palmitate extended-release injectable suspension.

There were no treatment-related effects on the offspring when pregnant rats were injected intramuscularly with 1-month paliperidone palmitate extended-release injectable suspension during the period of organogenesis at doses up to 250 mg/kg, which is ˜10 times the MRHD of 234 mg of the 1-month paliperidone palmitate extended-release injectable suspension based on mg/m² body surface area.

In animal reproduction studies, there were no increases in fetal abnormalities when pregnant rats and rabbits were treated orally with paliperidone during the period of organogenesis with up to 8 times the oral MRHD of 12 mg based on mg/m² body surface area.

Additional reproduction toxicity studies were conducted with orally administered risperidone, which is extensively converted to paliperidone. Cleft palate was observed in the offspring of pregnant mice treated with risperidone at 3 to 4 times the MRHD of 16 mg based on mg/m² body surface area; maternal toxicity occurred at 4 times the MRHD. There was no evidence of teratogenicity in embryo-fetal developmental toxicity studies with risperidone in rats and rabbits at doses up to 6 times the MRHD of 16 mg/day risperidone based on mg/m² body surface area. When the offspring of pregnant rats, treated with risperidone at 0.6 times the MRHD based on mg/m² body surface area, reached adulthood, learning was impaired. Increased neuronal cell death occurred in the fetal brains of the offspring of pregnant rats treated at 0.5 to 1.2 times the MRHD; the postnatal development and growth of the offspring was delayed.

In rat reproduction studies with risperidone, pup deaths occurred at oral doses which are less than the MRHD of risperidone based on mg/m² body surface area; it is not known whether these deaths were due to a direct effect on the fetuses or pups or, to effects on the dams.

8.2 Lactation

Risk Summary

Limited data from published literature report the presence of paliperidone in human breast milk. There is no information on the effects on the breastfed infant, or the effects on milk production; however, there are reports of sedation, failure to thrive, jitteriness, and extrapyramidal symptoms (tremors and abnormal muscle movements) in breastfed infants exposed to paliperidone's parent compound, risperidone (see Clinical Considerations). Paliperidone has been detected in plasma in adult subjects up to 18 months after a single-dose administration of 3-month paliperidone palmitate extended-release injectable suspension. The clinical significance on the breastfed infant is not known [see Clinical Pharmacology (12.3)]. The developmental and health benefits of breastfeeding should be considered along with the mother's clinical need for INVEGA HAFYERA and any potential adverse effects on the breastfed child from INVEGA HAFYERA or from the mother's underlying condition.

Clinical Considerations

Infants exposed to INVEGA HAFYERA through breastmilk should be monitored for excess sedation, failure to thrive, jitteriness, and extrapyramidal symptoms (tremors and abnormal muscle movements).

8.3 Females and Males of Reproductive Potential

Infertility

Females

Based on the pharmacologic action of paliperidone (D₂ receptor antagonism), treatment with INVEGA HAFYERA may result in an increase in serum prolactin levels, which may lead to a reversible reduction in fertility in females of reproductive potential [see Warnings and Precautions (5.10)].

8.4 Pediatric Use

Safety and effectiveness of INVEGA HAFYERA in patients less than 18 years of age have not been established. Use of INVEGA HAFYERA is not recommended in pediatric patients because of the potential longer duration of an adverse event. In clinical trials of oral paliperidone, there were notably higher incidences of dystonia, hyperkinesia, tremor, and parkinsonism in the adolescent population as compared to the adult studies.

Juvenile Animal Studies

No juvenile animal studies were conducted with the 6-month paliperidone palmitate extended-release injectable suspension.

In a study in which juvenile rats were treated with oral paliperidone from days 24 to 73 of age, a reversible impairment of performance in a test of learning and memory was seen, in females only, with a no-effect dose of 0.63 mg/kg/day, which produced plasma levels (AUC) of paliperidone similar to those in adolescents dosed at 12 mg/day. No other consistent effects on neurobehavioral or reproductive development were seen up to the highest dose tested (2.5 mg/kg/day), which produced plasma levels of paliperidone 2-3 times those in adolescents.

Juvenile dogs were treated for 40 weeks with oral risperidone, which is extensively metabolized to paliperidone in animals and humans, at doses of 0.31, 1.25, or 5 mg/kg/day. Decreased bone length and density were seen with a no-effect dose of 0.31 mg/kg/day, which produced plasma levels (AUC) of risperidone plus paliperidone which were similar to those in children and adolescents receiving the MRHD of risperidone. In addition, a delay in sexual maturation was seen at all doses in both males and females. The above effects showed little or no reversibility in females after a 12-week drug-free recovery period.

8.5 Geriatric Use

The clinical study of INVEGA HAFYERA did not include sufficient numbers of subjects aged 65 and over to determine whether they respond differently from younger subjects. Other reported clinical experience has not identified differences in responses between the elderly and younger patients.

This drug is substantially excreted by the kidney and clearance is decreased in patients with renal impairment [see Clinical Pharmacology (12.3)]. Because elderly patients are more likely to have decreased renal function, INVEGA HAFYERA is not recommended to be used in elderly patients with mild, moderate or severe renal impairment (see Use in Specific Populations (8.6)]

8.6 Renal Impairment

Use of INVEGA HAFYERA is not recommended for use in patients with mild, moderate, or severe renal impairment (creatinine clearance <90 mL/min) because necessary dosage adjustment is not possible with available strengths of INVEGA HAFYERA [Clinical Pharmacology (12.3)].

8.7 Hepatic Impairment

INVEGA HAFYERA has not been studied in patients with hepatic impairment. Based on a study with oral paliperidone, no dose adjustment is required in patients with mild or moderate hepatic impairment. Paliperidone has not been studied in patients with severe hepatic impairment [see Clinical Pharmacology (12.3)].

8.8 Patients with Parkinson's Disease or Lewy Body Dementia

Patients with Parkinson's Disease or Dementia with Lewy Bodies can experience increased sensitivity to INVEGA HAFYERA. Manifestations can include confusion, obtundation, postural instability with frequent falls, extrapyramidal symptoms, and clinical features consistent with neuroleptic malignant syndrome.

9 Drug Abuse and Dependence

9.1 Controlled Substance

INVEGA HAFYERA contains paliperidone, which is not a controlled substance.

9.2 Abuse

Paliperidone has not been systematically studied in animals or humans for its potential for abuse.

9.3 Dependence

Paliperidone has not been systematically studied in animals or humans for its potential for tolerance or physical dependence.

10 Overdosage

Human Experience

No cases of overdose were reported in premarketing studies with paliperidone palmitate injection.

While experience with paliperidone overdose is limited, among the few cases of overdose reported in premarketing trials with oral paliperidone, the highest estimated ingestion was 405 mg. Observed signs and symptoms included extrapyramidal symptoms and gait unsteadiness. Other potential signs and symptoms include those resulting from an exaggeration of paliperidone's known pharmacological effects, i.e., drowsiness and sedation, tachycardia and hypotension, and QT prolongation. Torsades de pointes and ventricular fibrillation have been reported in a patient in the setting of overdose with oral paliperidone.

Paliperidone is the major active metabolite of risperidone. Refer to the OVERDOSAGE section of the risperidone prescribing information for overdose experience with risperidone.

Management of Overdosage

Contact a Certified Poison Control Center for the most up to date information on the management of paliperidone and INVEGA HAFYERA overdosage (1-800-222-1222 or www.poison.org). Provide supportive care, including close medical supervision and monitoring. Treatment should consist of general measures employed in the management of overdosage with any drug. Consider the possibility of multiple drug overdosage. Ensure an adequate airway, oxygenation, and ventilation. Monitor cardiac rhythm and vital signs. Use supportive and symptomatic measures. There is no specific antidote to paliperidone.

Consider the extended-release characteristics of INVEGA HAFYERA and the half-life of paliperidone when assessing treatment needs and recovery.

11 Description

INVEGA HAFYERA™ contains a racemic mixture of (+)- and (−)-paliperidone palmitate. Paliperidone palmitate is an atypical antipsychotic belonging to the chemical class of benzisoxazole derivatives. The chemical name is (9RS)-3-[2-[4-(6-Fluoro-1,2-benzisoxazol-3-yl)piperidin-l-yl]ethyl]-2-methyl-4-oxo-6,7,8,9-tetrahydro-4H-pyrido[1,2-a]pyrimadin-9-yl hexadecanoate. Its molecular formula is C₃₉H₅₇FN₄0₄ and its molecular weight is 664.89. The structural formula is:

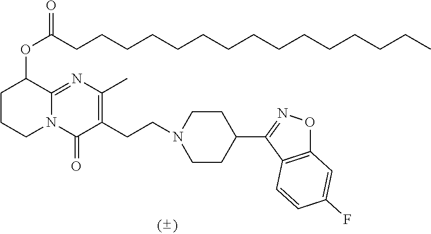

Paliperidone palmitate is very slightly soluble in ethanol and methanol, practically insoluble in polyethylene glycol 400 and propylene glycol, and slightly soluble in ethyl acetate.

INVEGA HAFYERA is available as a white to off-white sterile aqueous extended-release suspension for intramuscular injection in dose strengths of 1,092 mg and 1,560 mg paliperidone palmitate. The drug product hydrolyzes to the active moiety, paliperidone, resulting in dose strengths of 700 mg, and 1,000 mg of paliperidone, respectively. The inactive ingredients are polysorbate 20 (10 mg/mL), polyethylene glycol 4000 (75 mg/mL), citric acid monohydrate (7.5 mg/mL), sodium dihydrogen phosphate monohydrate (6 mg/mL), sodium hydroxide (5.4 mg/mL), and water for injection.

INVEGA HAFYERA is provided in a single-dose prefilled syringe (cyclic-olefin-copolymer) prefilled with either 700 mg (3.5 mL), or 1,000 mg (5.0 mL) paliperidone (as 1,092 mg, or 1,560 mg paliperidone palmitate) suspension with a tip cap, plunger rod, backstop and a thin walled 20 G, 1½-inch safety needle.

12 Clinical Pharmacology

12.1 Mechanism of Action

Paliperidone palmitate is hydrolyzed to paliperidone [see Clinical Pharmacology (12.3)]. Paliperidone is the major active metabolite of risperidone. The mechanism of action of paliperidone is unclear. However, its efficacy in the treatment of schizophrenia could be mediated through a combination of central dopamine D₂ and serotonin 5HT_{2A} receptor antagonism.

12.2 Pharmacodynamics

In vitro, paliperidone acts as an antagonist at the central dopamine D₂ and serotonin 5HT_{2A} receptors with binding affinities (Ki values) of 1.6-2.8 nM and 0.8-1.2 nM, respectively. Paliperidone also acts as an antagonist at histamine H₁ and α₁ and α₂ adrenergic receptors with bindings affinities of 32, 4, and 17 nM, respectively. Paliperidone has no appreciable affinity for cholinergic muscarinic or β₁- and β₂-adrenergic receptors. The pharmacological activity of the (+)- and (−)-paliperidone enantiomers is qualitatively and quantitatively similar in vitro.

12.3 Pharmacokinetics

The pharmacokinetics for INVEGA HAFYERA presented below are based on gluteal administration only.

INVEGA HAFYERA delivers paliperidone over a 6-month period, compared to the 1-month or 3-month products which are administered every month or every three months, respectively. INVEGA HAFYERA doses of 1,092 mg and 1,560 mg result in paliperidone total exposure ranges that are encompassed within the exposure range for corresponding doses of 1-month paliperidone palmitate injections (PP1M) (156 mg and 234 mg) or corresponding doses of 3-month paliperidone palmitate (PP3M) injections (546 mg and 819 mg, respectively) or to corresponding once daily doses of paliperidone extended-release tablets. However, mean trough concentrations (C_{trough}) at the end of the dosing interval were approximately 20-25% lower for INVEGA HAFYERA as compared to corresponding doses of 3-month paliperidone palmitate. The mean peak concentration (Cₘₐₓ) was higher (1.4 to 1.5-fold) for INVEGA HAFYERA as compared to corresponding doses of 3-month paliperidone palmitate.

Inter-subject variability in paliperidone PK parameters for INVEGA HAFYERA ranged from 42 to 48% for AUC_{6 months} and ranged from 56 to 103% for Cₘₐₓ. Because of the difference in pharmacokinetic profiles among the four paliperidone products, caution should be exercised when making a direct comparison of their pharmacokinetic properties.

Absorption

Due to its extremely low water solubility, the 6-month formulation of paliperidone palmitate dissolves slowly after intramuscular injection before being hydrolyzed to paliperidone and absorbed into the systemic circulation. The release of the drug starts as early as day 1 and is predicted to last longer than 18 months.

Following gluteal injection(s) of INVEGA HAFYERA at doses of 1,092 or 1,560 mg plasma concentrations of paliperidone rise to reach maximum concentrations at a median Tmax of 29 to 32 days. The release profile and dosing regimen of INVEGA HAFYERA results in sustained concentrations over 6 months. The total and peak dose-normalized exposures of paliperidone following INVEGA HAFYERA administration were comparable between 1,092 mg and 1,560 mg dose levels. The median steady-state peak:trough ratio for an INVEGA HAFYERA dose is 3.1 and 3.0 following gluteal administration of 1,092 and 1,560 mg respectively.

Distribution

Following administration of INVEGA HAFYERA, the apparent volume of distribution of paliperidone is 1,960 L.

The plasma protein binding of racemic paliperidone is 74%.

Elimination

Metabolism

In a study with oral immediate-release ¹⁴C-paliperidone, one week following administration of a single oral dose of 1 mg immediate-release ¹⁴C-paliperidone, 59% of the dose was excreted unchanged into urine, indicating that paliperidone is not extensively metabolized in the liver. Approximately 80% of the administered radioactivity was recovered in urine and 11% in the feces. Four metabolic pathways have been identified in vivo, none of which accounted for more than 10% of the dose: dealkylation, hydroxylation, dehydrogenation, and benzisoxazole scission. Although in vitro studies suggested a role for CYP2D6 and CYP3A4 in the metabolism of paliperidone, there is no evidence in vivo that these isozymes play a significant role in the metabolism of paliperidone. Population pharmacokinetics analyses indicated no discernible difference on the apparent clearance of paliperidone after administration of oral paliperidone between extensive metabolizers and poor metabolizers of CYP2D6 substrates.

Excretion

The median apparent half-life of paliperidone following a single INVEGA HAFYERA of either 1,092 or 1,560 mg was 148 and 159 days respectively. the concentration of paliperidone remaining in the circulation 18 months after dosing of 1,560 mg 6-month paliperidone palmitate extended-release injectable suspension stopped is estimated to be 18% of the average steady-state levels.

Drug Interaction Studies

No specific drug interaction studies have been performed with INVEGA HAFYERA. The information below is obtained from studies with oral paliperidone.

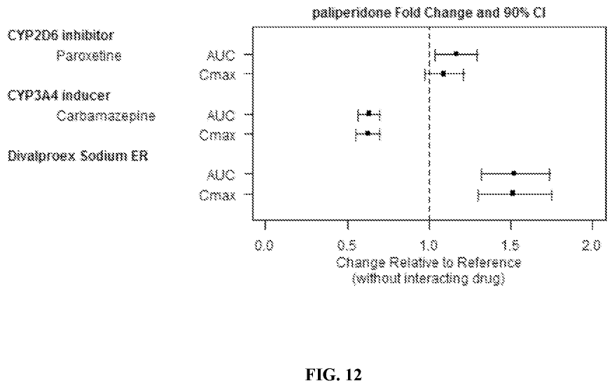

Effects of other drugs on the exposures of INVEGA HAFYERA are summarized in FIG. 12. After oral administration of 20 mg/day of paroxetine (a potent CYP2D6 inhibitor), an increase in mean Cₘₐₓ and AUC values at steady-state was observed (see FIG. 12). Higher doses of paroxetine have not been studied. The clinical relevance is unknown. After oral administration of paliperidone, a decrease in mean Cₘₐₓ and AUC values at steady state is expected when patients are treated with carbamazepine, a strong inducer of both CYP3A4 and P-gp [see Drug Interactions (7.1)]. This decrease is caused, to a substantial degree, by a 35% increase in renal clearance of paliperidone.

In vitro studies indicate that CYP2D6 and CYP3A4 may be involved in paliperidone metabolism, however, there is no evidence in vivo that inhibitors of these enzymes significantly affect the metabolism of paliperidone; they contribute to only a small fraction of total body clearance. In vitro studies demonstrated that paliperidone is a substrate of P-glycoprotein (P-gp) [see Drug Interactions (7.2)].

Co-administration of a single dose of an oral paliperidone extended-release tablet 12 mg with divalproex sodium extended-release tablets (two 500 mg tablets once daily) resulted in an increase of approximately 50% in the Cₘₐₓ and AUC of paliperidone. Since no significant effect on the systemic clearance was observed, a clinically significant interaction would not be expected between divalproex sodium extended-release tablets and INVEGA HAFYERA. This interaction has not been studied with INVEGA HAFYERA.

In vitro studies in human liver microsomes demonstrated that paliperidone does not substantially inhibit the metabolism of drugs metabolized by cytochrome P450 isozymes, including CYP1A2, CYP2A6, CYP2C8/9/10, CYP2D6, CYP2E1, CYP3A4, and CYP3A5. Therefore, paliperidone is not expected to inhibit clearance of drugs that are metabolized by these metabolic pathways in a clinically relevant manner. Paliperidone is also not expected to have enzyme inducing properties.

Paliperidone is a weak inhibitor of P-gp at high concentrations. No in vivo data are available, and the clinical relevance is unknown.

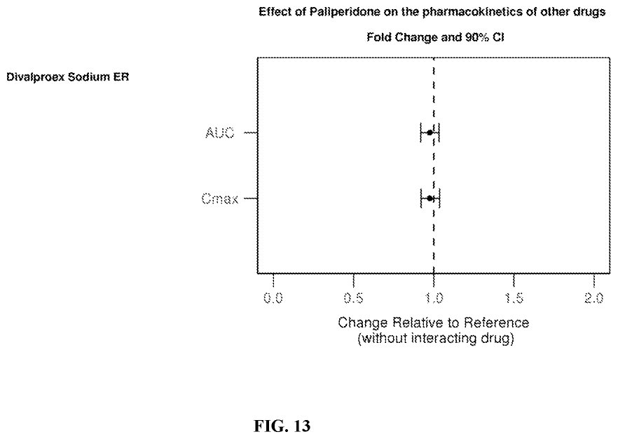

The effects of INVEGA HAFYERA on the exposures of other drugs are summarized in FIG. 13.

After oral administration of paliperidone, the steady-state Cₘₐₓ and AUC of valproate were not affected in 13 patients stabilized on valproate. In a clinical study, subjects on stable doses of valproate had comparable valproate average plasma concentrations when oral paliperidone extended-release tablets 3-15 mg/day was added to their existing valproate treatment [see Drug Interactions (7.1)].

Specific Populations

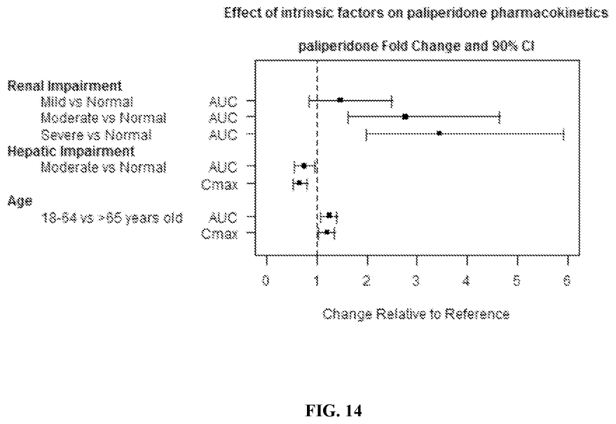

No specific pharmacokinetic studies have been performed with INVEGA HAFYERA in specific populations. All the information is obtained from studies with oral paliperidone or is based on the population pharmacokinetic modelling of oral paliperidone and INVEGA HAFYERA. Exposures of paliperidone in specific populations (renal impairment, hepatic impairment and elderly) are summarized in FIG. 14 [see Dosage and Administration (2.5) and Use in Specific Populations (8.6)].

Patients with Hepatic Impairment

After oral administration of paliperidone in patients with moderate hepatic impairment, the plasma concentrations of free paliperidone were similar to those of healthy subjects, although total paliperidone exposure decreased because of a decrease in protein binding. Paliperidone has not been studied in patients with severe hepatic impairment [see Use in Specific Populations (8.7)].

Geriatric Patients

After oral administration of paliperidone in elderly subjects, the Cₘₐₓ and AUC increased 1.2-fold compared to young subjects. This may be attributable to age-related decreases in creatinine clearance [see Dosage and Administration (2.5) and Use in Specific Populations (8.5)].

Smokers

Based on in vitro studies utilizing human liver enzymes, paliperidone is not a substrate for CYP1A2; smoking should, therefore, not have an effect on the pharmacokinetics of paliperidone.

Male and Female Patients

Slower absorption was observed in females in a population pharmacokinetic analysis. At apparent steady-state with 6-month paliperidone palmitate extended-release injectable suspension the trough concentrations were similar between males and females.

Obese Patients

Lower Cₘₐₓ was observed in overweight and obese subjects. At apparent steady state with INVEGA HAFYERA, the trough concentrations were similar among normal, overweight, and obese subjects.

13 Nonclinical Toxicology

13.1 Carcinogenesis, Mutagenesis, Impairment of Fertility

Carcinogenesis

No carcinogenicity studies were conducted with the 6-month paliperidone palmitate extended-release injectable suspension.

The carcinogenic potential of intramuscularly injected 1-month paliperidone palmitate extended-release injectable suspension was assessed in rats. There was an increase in mammary gland adenocarcinomas in female rats at 16, 47, and 94 mg/kg/month, which are ˜0.7, 2 and 4 times, respectively, the MRHD of 234 mg of the 1-month paliperidone palmitate extended-release suspension based on mg/m² body surface area. A no-effect dose was not established. Male rats showed an increase in mammary gland adenomas, fibroadenomas, and carcinomas at ˜2 and 4 times the MRHD of 234 mg of the 1-month paliperidone palmitate extended-release suspension based on mg/m² body surface area. A carcinogenicity study in mice has not been conducted with paliperidone palmitate.

Carcinogenicity studies with risperidone, which is extensively converted to paliperidone in rats, mice, and humans, were conducted in Swiss albino mice and Wistar rats. Risperidone was administered in the diet at daily doses of 0.63, 2.5, and 10 mg/kg for 18 months to mice and for 25 months to rats. A maximum tolerated dose was not achieved in male mice. There were statistically significant increases in pituitary gland adenomas, endocrine pancreas adenomas, and mammary gland adenocarcinomas. The no-effect dose for these tumors was less than or equal to the maximum recommended human dose of risperidone based on mg/m² body surface area (see risperidone package insert). An increase in mammary, pituitary, and endocrine pancreas neoplasms has been found in rodents after chronic administration of other antipsychotic drugs and is considered to be mediated by prolonged dopamine D₂ antagonism and hyperprolactinemia. The relevance of these tumor findings in rodents to human risk is unclear [see Warnings and Precautions (5.7)].

Mutagenesis

Paliperidone palmitate showed no genotoxicity in the in vitro Ames bacterial reverse mutation test or the mouse lymphoma assay. Paliperidone was not genotoxic in the in vitro Ames bacterial reverse mutation test, the mouse lymphoma assay or the in vivo rat bone marrow micronucleus test.

Impairment of Fertility

No fertility studies were conducted with the 6-month paliperidone palmitate extended-release injectable suspension.

In an oral paliperidone study of fertility, the percentage of treated female rats that became pregnant was not affected at oral doses of paliperidone of up to 2.5 mg/kg/day which is 2 times the oral MRHD of 12 mg based on mg/m² body surface area. However, pre- and post-implantation loss was increased, and the number of live embryos was slightly decreased, at 2.5 mg/kg, a dose that also caused slight maternal toxicity. These parameters were not affected at an oral dose of 0.63 mg/kg, which is half of the oral MRHD of 12 mg based on mg/m² body surface area.

The fertility of male rats was not affected at oral doses of paliperidone of up to 2 times the oral MRHD of 12 mg/day based on mg/m² body surface area, although sperm count and sperm viability studies were not conducted with paliperidone. In a subchronic study in Beagle dogs with risperidone, which is extensively converted to paliperidone in dogs and humans, all doses tested (0.31 mg/kg-5.0 mg/kg) resulted in decreases in serum testosterone and in sperm motility and concentration (0.6 to 10 times the MRHD of 16 mg/day for risperidone, based on mg/m² body surface area). Serum testosterone and sperm parameters partially recovered, but remained decreased after the last observation (two months after treatment was discontinued).

13.2 Animal Toxicology and/or Pharmacology

Injection site toxicity was assessed in minipigs injected intramuscularly with the 6-month paliperidone palmitate extended-release injectable suspension at doses up to 2,115 mg, which is slightly above the MRHD. Injection site inflammatory reactions were greater and more advanced than reactions to the 1-month paliperidone palmitate extended-release injectable suspension. Reversibility of these findings was not examined.

14 Clinical Studies

The efficacy of INVEGA HAFYERA for the treatment of schizophrenia in patients who had previously been stably treated with either PP1M for at least 4 months or PP3M for at least one 3-month injection cycle was evaluated in a randomized, double-blind, active-controlled, interventional, parallel-group, multicenter, non-inferiority study designed to evaluate time to relapse in adults with a DSM-5 diagnosis of schizophrenia.

Patients could enter the study if previously treated with PP1M at dosages of 156 or 234 mg, PP3M at dosages of 546 or 819 mg, injectable risperidone at dosages of 50 mg, or any oral antipsychotic with a reason to change (e.g., efficacy, safety, tolerability, or a preference for a long-acting injectable medication) and with a PANSS total score of <70 points.

After establishing tolerability with PP1M (at dosages of 156 or 234 mg) or PP3M (at dosages of 546 or 819 mg) and clinical stability, defined by having a PANSS total score of <70 points for the previous 2 assessments prior to the double-blind phase, patients were randomized in a 2:1 ratio to receive INVEGA HAFYERA (478 patients) or PP3M (224 patients).

The primary efficacy variable was time to first relapse in the double-blind phase. The primary efficacy analysis was based on the difference in Kaplan-Meier 12-month estimates of percentage of subjects remaining relapse-free between INVEGA HAFYERA and 3-month paliperidone palmitate extended-release injectable suspension. Relapse was pre-defined as emergence of one or more of the following: psychiatric hospitalization, ≥25% increase (if the baseline score was >40) or a 10-point increase (if the baseline score was ≤40) in total PANS S score on two consecutive assessments, deliberate self-injury, violent behavior, suicidal/homicidal ideation: a score of ≥5 (if the maximum baseline score was ≤3) or ≥6 (if the maximum baseline score was 4) on two consecutive assessments of the specific PANS S items.

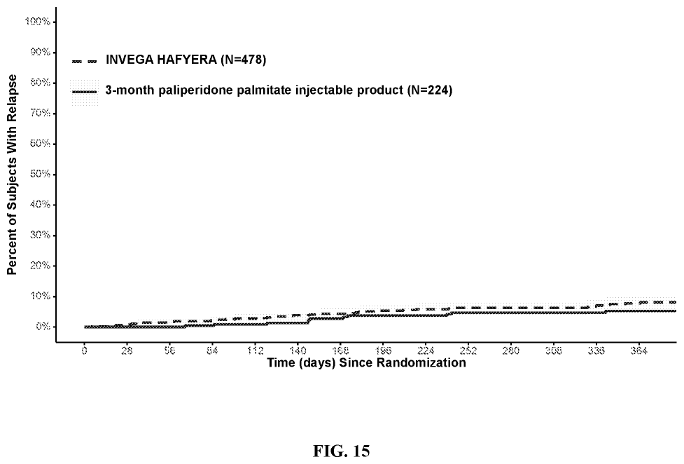

A relapse event was experienced by 7.5% and 4.9% of patients in the INVEGA HAFYERA and PP3M treatment groups, respectively, with the Kaplan-Meier estimated difference (INVEGA HAFYERA−PP3M) of 2.9% (95% CI: −1.1 to 6.8). The upper bound of the 95% CI (6.8%) was less than 10%, the prespecified non-inferiority margin. The study demonstrated non-inferiority of INVEGA HAFYERA to PP3M. A Kaplan-Meier plot of time to relapse by treatment group is shown in FIG. 15.

An evaluation of population subgroups did not reveal any clinically significant differences in responsiveness on the basis of gender, age, or race.

16 How Supplied/Storage and Handling

INVEGA HAFYERA™ is available as a white to off-white sterile aqueous extended-release suspension for gluteal intramuscular injection in dose strengths of 1,092 mg/3.5 mL and 1,560 mg/5 mL paliperidone palmitate. The kit contains a single-dose prefilled syringe and a 20 G, 1½-inch safety needle.

1,092 mg paliperidone palmitate kit (NDC 50458-611-01)

1,560 mg paliperidone palmitate kit (NDC 50458-612-01)

Storage and Handling

Store at room temperature 20° C. to 25° C. (68° F. to 77° F.); excursions between 15° C. and 30° C. (59° F. and 86° F.) are permitted. Do not mix with any other product or diluent.

Ship and store in a horizontal position. See arrows on product carton for proper orientation.

17 Patient Counseling Information

Advise the patient to read the FDA-approved patient labeling (Patient Information).

Neuroleptic Malignant Syndrome (NMS)

Counsel patients about a potentially fatal side effect referred to as Neuroleptic Malignant Syndrome (NMS) that has been reported in association with administration of antipsychotic drugs. Patients should contact their health care provider or report to the emergency room if they experience the following signs and symptoms of NMS, including hyperpyrexia, muscle rigidity, altered mental status, and evidence of autonomic instability (irregular pulse or blood pressure, tachycardia, diaphoresis, and cardiac dysrhythmia [see Warnings and Precautions (5.3)].

Tardive Dyskinesia

Counsel patients on the signs and symptoms of tardive dyskinesia and to contact their health care provider if these abnormal movements occur [see Warnings and Precautions (5.5)].

Metabolic Changes

Educate patients about the risk of metabolic changes, how to recognize symptoms of hyperglycemia (high blood sugar) and diabetes mellitus (e.g., polydipsia, polyuria, polyphagia, and weakness), and the need for specific monitoring, including blood glucose, lipids, and weight [see Warnings and Precautions (5.6)].

Orthostatic Hypotension

Educate patients about the risk of orthostatic hypotension, particularly at the time of initiating treatment, re-initiating treatment, or increasing the dose [see Warnings and Precautions (5.7)].

Leukopenia/Neutropenia

Advise patients with a pre-existing low WBC or a history of drug induced leukopenia/neutropenia they should have their CBC monitored while taking INVEGA HAFYERA [see Warnings and Precautions (5.9)].

Hyperprolactinemia

Counsel patients on signs and symptoms of hyperprolactinemia that may be associated with chronic use of INVEGA HAFYERA. Advise them to seek medical attention if they experience any of the following: amenorrhea or galactorrhea in females, erectile dysfunction or gynecomastia in males. [See Warnings and Precautions (5.10)].

Interference with Cognitive and Motor Performance

As INVEGA HAFYERA has the potential to impair judgment, thinking, or motor skills, caution patients about operating hazardous machinery, including automobiles, until they are reasonably certain that INVEGA HAFYERA therapy does not affect them adversely [see Warnings and Precautions (5.11)].

Priapism

Advise patients of the possibility of painful or prolonged penile erections (priapism). Instruct the patient to seek immediate medical attention in the event of priapism [see Warnings and Precautions (5.14)].

Heat Exposure and Dehydration

Counsel patients on the importance of avoiding overheating and dehydration [see Warnings and Precautions (5.15)].

Concomitant Medication

Advise patients to inform their health care providers if they are taking, or plan to take any prescription or over-the-counter drugs, as there is a potential for interactions [see Drug Interactions (7)].

Pregnancy

Advise patients to notify their healthcare provider if they become pregnant or intend to become pregnant during treatment with INVEGA HAFYERA. Advise patients that INVEGA HAFYERA may cause extrapyramidal and/or withdrawal symptoms in a neonate. Advise patients that there is a pregnancy registry that monitors pregnancy outcomes in women exposed to INVEGA HAFYERA during pregnancy [see Use in Specific Populations (8.1)].

Lactation

Advise breastfeeding women using INVEGA HAFYERA to monitor infants for somnolence, failure to thrive, jitteriness, and extrapyramidal symptoms (tremors and abnormal muscle movements) and to seek medical care if they notice these signs [see Use in Specific Populations (8.2)].

Infertility

Advise females of reproductive potential that INVEGA HAFYERA may impair fertility due to an increase in serum prolactin levels. The effects on fertility are reversible [see Use in Specific Populations (8.3)].

INVEGA HAFYERA (paliperidone palmitate) Extended-Release Injectable Suspension

INVEGA TRINZA® INVEGA SUSTENNA®, RISPERDAL®, and RISPERDAL CONSTAR® are trademarks of Janssen Pharmaceuticals, Inc.

Product of Ireland

Manufactured by:
Janssen Pharmaceutica N.V.
Beerse, Belgium

Manufactured for:
Janssen Pharmaceuticals, Inc.
Titusville, N.J. 08560

©2021 Janssen Pharmaceutical Companies

Patient Information
INVEGA HAFYERA™ (in-VAY-guh HAF-ye-RA)
(paliperidone palmitate) extended-release injectable suspension

What is the Most Important Information I Should Know About INVEGA HAFYERA?

INVEGA HAFYERA may cause serious side effects, including:
Increased risk of death in elderly people with dementia-related psychosis. INVEGA HAFYERA increases the risk of death in elderly people who have lost touch with reality (psychosis) due to confusion and memory loss (dementia). INVEGA HAFYERA is riot for the treatment of people with dementia-related psychosis.

What is INVEGA HAFYERA?

INVEGA HAFYERA is a prescription medicine given by injection by a healthcare provider 1 time every 6 months and used for the treatment of schizophrenia in adults who have been adequately treated with either:
A 1 time each month paliperidone palmitate extended-release injectable suspension for at least 4 months.
A 1 time every 3 months paliperidone palmitate extended-release injectable suspension for at least 3 months.
It is not known if INVEGA HAFYERA is safe and effective in children under 18 years of age.

Do not receive INVEGA HAFYERA if you are allergic to paliperidone palmitate, risperidone, or any of the ingredients in INVEGA HAFYERA. See the end of this Patient Information leaflet for a complete list of ingredients in INVEGA HAFYERA.

Before receiving INVEGA HAFYERA, tell your healthcare provider about all your medical conditions, including if you:
have had Neuroleptic Malignant Syndrome (NMS)
have or have had heart problems, including a heart attack, heart failure, abnormal heart rhythm, or long QT syndrome
have or have had low levels of potassium or magnesium in your blood
have or have had uncontrolled movements of your tongue, face, mouth, or jaw (tardive dyskinesia)
have or have had kidney or liver problems
have diabetes or have a family history of diabetes
have Parkinson's disease or a type of dementia called Lewy Body Dementia
have had a low white blood cell count
have had problems with dizziness or fainting or are being treated for high blood pressure
have or have had seizures or epilepsy
are pregnant or plan to become pregnant. It is not known if INVEGA HAFYERA will harm your unborn baby.
   Tell your healthcare provider right away if you become pregnant or think you may be pregnant during treatment with INVEGA HAFYERA.
   If you become pregnant while receiving INVEGA HAFYERA, talk to your healthcare provider about registering with the National Pregnancy Registry for Atypical Antipsychotics. You can register by calling 1-866-961-2388 or visit http://womensmentalhealth.org/clinical-and-research-programs/pregnancyregistry/.
   Babies born to mothers who receive INVEGA HAFYERA during their third trimester of pregnancy may develop agitation, low muscle tone (floppy baby syndrome) tremors, excessive sleepiness, breathing problems, and feeding problems. Tell your healthcare provider right away if your baby develops any of these symptoms.
are breastfeeding or plan to breastfeed. INVEGA HAFYERA can pass into your breast milk. Talk to your healthcare provider about the best way to feed your baby during treatment with INVEGA HAFYERA.
Tell your healthcare provider about all the medicines you take, including prescription and over-the-counter medicines, vitamins, and herbal supplements.

INVEGA HAFYERA and other medicines may affect each other causing possible serious side effects. INVEGA HAFYERA may affect the way other medicines work, and other medicines may affect how INVEGA HAFYERA works.

Your healthcare provider can tell you if it is safe to receive INVEGA HAFYERA with your other medicines. Do not start or stop any medicines during treatment with INVEGA HAFYERA without talking to your healthcare provider first.

Know the medicines you take. Keep a list of them to show to your healthcare provider or pharmacist when you get a new medicine.

How Will I Receive INVEGA HAFYERA?
Follow your INVEGA HAFYERA treatment schedule exactly as your healthcare provider tells you to.
Your healthcare provider will tell you how much INVEGA HAFYERA you will receive and when you will receive it.
INVEGA HAFYERA is given as an injection by your healthcare provider into the muscle (intramuscularly) of your buttocks, 1 time every 6 months.

What Should I Avoid while Receiving INVEGA HAFYERA?
Do not drive, operate heavy machinery, or do other dangerous activities until you know how INVEGA HAFYERA affects you. INVEGA HAFYERA may affect your judgment, thinking, or motor skills.
Avoid getting too hot or dehydrated.
   Do not exercise too much.
   In hot weather, stay inside in a cool place if possible.
   Stay out of the sun.
   Do not wear too much clothing or heavy clothing.
   Drink plenty of water.

What are the Possible Side Effects of INVEGA HAFYERA?

INVEGA HAFYERA may cause serious side effects, including:
See “What is the most important information I should know about INVEGA HAFYERA?”
Cerebrovascular problems (including stroke) in elderly people with dementia-related psychosis that can lead to death.
Neuroleptic Malignant Syndrome (NMS), a serious condition that can lead to death. Call your healthcare provider or go to your nearest hospital emergency room right away if you have some or all of the following signs and symptoms of NMS:
   high fever
   stiff muscles
   confusion
   sweating
   changes in your breathing, pulse, heart rate, and blood pressure
Problems with your heartbeat. These heart problems can cause death. Call your healthcare provider right away if you have any of these symptoms:
   passing out or feeling like you will pass out
   dizziness
   feeling as if your heart is pounding or missing beats
Uncontrolled body movements (tardive dyskinesia). INVEGA HAFYERA may cause movements that you cannot control in your face, tongue, or other body parts. Tardive dyskinesia may not go away, even if you stop receiving INVEGA HAFYERA. Tardive dyskinesia may also start after you stop receiving INVEGA HAFYERA.
Problems with your metabolism such as:
   high blood sugar (hyperglycemia) and diabetes. Increases in blood sugar can happen in some people who receive INVEGA HAFYERA. Extremely high blood sugar can lead to coma or death. Your healthcare provider should check your blood sugar before you start and regularly during treatment with INVEGA HAFYERA.
   Call your healthcare provider if you have any of these symptoms of high blood sugar during treatment with INVEGA HAFYERA:
      feel very thirsty
      need to urinate more than usual
      feel very hungry
      feel weak or tired
      feel sick to your stomach
      feel confused, or your breath smells fruity
   increased fat levels (cholesterol and triglycerides) in your blood. Your healthcare provider should check the fat levels in your blood before you start and regularly during treatment with INVEGA HAFYERA.
   weight gain. You and your healthcare provider should check your weight before you start and often during treatment with INVEGA HAFYERA.
Decreased blood pressure (orthostatic hypotension) and fainting. You may feel lightheaded or faint when you rise too quickly from a sitting or lying position, especially early in treatment or when the dose is changed.
Falls. INVEGA HAFYERA may make you sleepy or dizzy, may cause a decrease in your blood pressure when changing position (orthostatic hypotension), and can slow your thinking and motor skills which may lead to falls that can cause fractures or other injuries.
Low white blood cell count. Your healthcare provider may do blood tests during the first few months of treatment with INVEGA HAFYERA.
Increased prolactin levels in your blood (hyperprolactinemia). INVEGA HAFYERA may cause a rise in the blood levels of a hormone called prolactin (hyperprolactinemia) that may cause side effects including missed menstrual periods, a reversible reduction in fertility in females who are able to become pregnant, leakage of milk from the breasts, development of breasts in men, or problems with erection.
INVEGA HAFYERA can make you sleepy or dizzy, and can slow your thinking and motor skills. Do not drive, operate heavy machinery, or do other dangerous activities until you know how INVEGA HAFYERA affects you.
Seizures (convulsions).
Difficulty swallowing that can cause food or liquid to get into your lungs.
Prolonged or painful erection lasting more than 4 hours (priapism). Call your healthcare provider or go to your nearest emergency room right away if you have an erection that lasts more than 4 hours.
Problems controlling your body temperature so that you feel too warm. See, “What should I avoid while receiving INVEGA HAFYERA?”

The Most Common Side Effects of INVEGA HAFYERA include:
upper respiratory tract infections
injection site reactions
weight gain
headache
feeling restlessness or difficulty sitting still
slow movements
tremors
stiffness
shuffling walk
These are not all the possible side effects of INVEGA HAFYERA. Call your doctor for medical advice about side effects. You may report side effects to FDA at 1-800-FDA-1088.

General Information About INVEGA HAFYERA.

Medicines are sometimes prescribed for purposes other than those listed in a Patient Information leaflet. You can ask your pharmacist or healthcare provider for information about INVEGA HAFYERA that is written for health professionals.

What are the Ingredients in INVEGA HAFYERA?
Active ingredient: paliperidone palmitate
Inactive ingredients: polysorbate 20, polyethylene glycol 4000, citric acid monohydrate, sodium dihydrogen phosphate monohydrate, sodium hydroxide, and water for injection
Manufactured by: Janssen Pharmaceutica N.V. Beerse, Belgium
Manufactured for Janssen Pharmaceuticals, Inc. Titusville, N.J. 08560

©2021 Janssen Pharmaceutical Companies
For more information, go to www.Invegahafyerahcp.com or call 1-800-526-7736. This Patient Information has been approved by the U.S. Food and Drug Administration. Revised: August 2021

### Signature Page 1 of 1

This is a representation of an electronic record that was signed electronically. Following this are manifestations of any and all electronic signatures for this electronic record.

/s/

BERNARD A FISCHER on behalf of TIFFANY R FARCHIONE

Aug. 30, 2021 07:20:45 PM

### Numbered Embodiments

1. A pharmaceutical product comprising a paliperidone palmitate extended-release injectable suspension having a six month dosing interval (PP6M), wherein the pharmaceutical product is packaged, and wherein the package includes a label that identifies the PP6M as a an approved drug product for the treatment of schizophrenia after a patient has been adequately treated with either a one-month paliperidone palmitate extended-release injectable suspension (PP1M) for at least four months or a three-month paliperidone palmitate extended-release injectable suspension (PP3M) following at least one 3-month injection cycle.
2. A method for treating schizophrenia in a patient in need thereof, comprising administering an approved drug product comprising PP6M in an amount and manner that is described in a drug product label for the approved drug product.
3. A method of selling an approved drug product comprising PP6M, said method comprising selling such drug product, wherein a drug product label for a reference listed drug for such drug product includes instructions for treating a patient with schizophrenia after the patient has been adequately treated with either PP1M for at least four months or PP3M following at least one 3-month injection cycle.
4. A method of offering for sale a drug product comprising PP6M, said method comprising offering for sale such drug product, wherein a drug product label for a reference listed drug for such drug product includes instructions for treating a patient with schizophrenia after the patient has been adequately treated with either PP1M for at least four months or PP3M following at least one 3-month injection cycle.
5. The method of any one of embodiments 2-4, wherein the drug product is an ANDA drug product, a supplemental New Drug Application drug product, or a 505(b)(2) drug product.
6. The method of any one of embodiments 2-5, wherein the drug product label comprises data directed to comparing PP6M to PP3M or a placebo.

What is claimed is:

1. An approved drug product in a prefilled syringe, wherein the approved drug product comprises an extended release injectable suspension of 1092 mg of paliperidone palmitate in a 3.5 ml volume or 1560 mg of paliperidone palmitate in a 5 ml volume.

2. The approved product of claim 1, comprising 1092 mg of paliperidone palmitate in a 3.5 ml volume.

3. The approved product of claim 1, comprising 1560 mg of paliperidone palmitate in a 5 ml volume.
